SEQUENCE LISTING (69 sequences)

SEQ ID NO: 1 - DNA, 1617 bp, Diabrotica virgifera
aaatcaagttacttagcaaaacgaaagctacttgctttgtagtttaatttttatgtgtattaaatagctcacagaaactgtgtattagtgattggatattgatagtttatattatcaaacattttgttccgtgtgtctatacataaaaacatgttttttggtaaatgtctggatacctcaaataataatacgtttttcaatttccacacatcccgagagatgaaggcagacgagaagccaccgattaagcctcaccaaagcaccttcgggtaccctgcgtttccaaacccagcgatgtttaccccatcacaaatcctaatggctagtcaactaatggcagcatccggcttgaccatatctggaaaccctgccttcttccatccaggtttgatgcccctaccatgggcagcaaactcacctccttccaattctccaccctcaccaccgaccagcaacgaaccgctgtcccctgcgatgagtacaagaaaacttagtcataataacaataatgttgttagtagtagtacgacgacagaaattaggaaaagaaagtataagttagaagaagaagccacgagtttaccgctgccatcccctacgtctagtgtatcgcctccttcggggtctgatattaaagatcctaatagggataagcagttcacctgtggagtatgtaatagatcatttggctacaaacacgtcctgcaaaatcacgaaaggacacataccggagagaaacctttcgaatgtccagagtgccacaaaaggttcaccagagaccatcaccttaagacccacatgagacttcatacaggagaacgcccttatcattgcgaacactgtgacagacaattcgtccaagttgccaaccttcgaagacatttgagggtgcatacaggagaacgaccttactcttgtgaacactgtactgccaaattctcagactccaatcagctcaaggctcacatacttatccataccaatgaaaaaccattttcgtgtgaaaggtgtcacagcaggttcagaaggaggcatcacttgttgcaccataaatgcggcatgcctgataaagaaatgactcctgatacagatgaagaggagttaaagaggaggagagtgctacattctctttctacgccaattattacgcttcctctccaagtagctctaccagaacaaaccgagcccgaagacctcagtatgtcgacggggatgcactcaaacaattcgggaagcagcggctccccgatcgtaagtccttgtttaaaagaagaagaggacgacatcgaaacaactgcgatatttctacaaagaaggccaatgagtagatcctagtctagttattttcgtgagatatacgggtatgttaaagtcaaattataattatccaaaatatatacatagtcccttatcacagcgtcacttccagaggaattgttttggggaataaaaaagtctaaaaggaaagaagtaactgttacacacttgcaaataatttctaatacatatattatatttacttgcactacaggtattgtgttgaattttttatagaacaatagatttcaaataacaagtaataaaatactcatttatattacagtggaaccccgataa

SEQ ID NO: 2 - DNA, 1640 bp, Diabrotica virgifera
ctgacagtcaataggtgtagttatacgacatttagatagatttgataaacatttacgacgacagcacgaatattttttattttcttttgtacatttttgtattttcgtggtgcgtgatgaagtgacaatggactgggtttgtgcgtggttttgattttgtttttgctatttcgatttttgtttttgaaatgccgcttctacaagaaaatactccaaaaagagatttccacacatcccgagagatgaaggcagacgagaagccaccgattaagcctcaccaaagcaccttcgggtaccctgcgtttccaaacccagcgatgtttaccccatcacaaatcctaatggctagtcaactaatggcagcatccggcttgaccatatctggaaaccctgccttcttccatccaggtttgatgcccctaccatgggcagcaaactcacctccttccaattctccaccctcaccaccgaccagcaacgaaccgctgtcccctgcgatgagtacaagaaaacttagtcataataacaataatgttgttagtagtagtacgacgacagaaattaggaaaagaaagtataagttagaagaagaagccaccagtttaccgctgccatcccctacgtctagtgtatcgcctccttcggggtctgatattaaagatcctaatagggataagcagttcacctgtggagtatgtaatagatcatttggctacaaacacgtcctgcaaaatcacgaaaggacacataccggagagaaacctttcgaatgtccagagtgccacaaaaggttcaccagagaccatcaccttaagacccacatgagacttcatacaggagaacgcccttatcattgcgaacactgtgacagacaattcgtccaagttgccaaccttcgaagacatttgagggtgcatacaggagaacgaccttactcttgtgaacactgtactgccaaattctcagactccaatcagctcaaggctcacatacttatccataccaatgaaaaaccattttcgtgtgaaaggtgtcacagcaggttcagaaggaggcatcacttgttgcaccataaatgcggcatgcctgataaagaaatgactcctgatacagatgaagaggagttaaagaggaggagagtgctacattctctttctacgccaattattacgcttcctctccaagtagctctaccagaacaaaccgagcccgaagacctcagtatgtcgacggggatgcactcaaacaattcgggaagcagcggctccccgatcgtaagtccttgtttaaaagaagaagaggacgacatcgaaacaactgcgatatttctacaaagaaggccaatgagtagatcctagtctagttattttcgtgagatatacgggtatgttaaagtcaaattataattatccaaaatatatacatagtcccttatcacagcgtcacttccagaggaattgttttggggaataaaaaagtctaaaaggaaagaagtaactgttacacacttgcaaataatttctaatacatatattatatttacttgcactacaggtcttgtgttgaattttttatagaacaatagatttcaaataacaagtaataaaatactcatttatattacagtggaaccccgataa

SEQ ID NO: 3 - protein, 371 aa, Diabrotica virgifera
MKADEKPPIKPHQSTFGYPAFPNPAMFTPSQILMASQLMAASGLTISGNPAFFHPGLMPLPWAANSPPSNSPPSPPTSNEPLSPAMSTRKLSHNNNNVVSSSTTTEIRKRKYKLEEEATSLPLPSPTSSVSPPSGSDIKDPNRDKQFTCGVCNRSFGYKHVLQNHERTHTGEKPFECPECHKRFTRDHHLKTHMRLHTGERPYHCEHCDRQFVQVANLRRHLRVHTGERPYSCEHCTAKFSDSNQLKAHILIHTNEKPFSCERCHSRFRRRHHLLHHKCGMPDKEMTPDTDEEELKRRRVLHSLSTPIITLPLQVALPEQTEPEDLSMSTGMHSNNSGSSGSPIVSPCLKEEEDDIETTAIFLQRRPMSRS

SEQ ID NO: 4 - DNA, 291 bp, Diabrotica virgifera
cctgtggagtatgtaatagatcatttggctacaaacacgtcctgcaaaatcacgaaaggacacataccggagagaaacctttcgaatgtccagagtgccacaaaaggttcaccagagaccatcaccttaagacccacatgagacttcatacaggagaacgcccttatcattgcgaacactgtgacagacaattcgtccaagttgccaaccttcgaagacatttgagggtgcatacaggagaacgaccttactcttgtgaacactgtactgccaaattctcagactccaatc

SEQ ID NO: 5 - DNA, 20 bp, Artificial Sequence
  promotor oligonucleotide
taatacgactcactataggg

SEQ ID NO: 6 - DNA, 39 bp, Artificial Sequence
  primer oligonucleotide
taatacgactcactatagggcctgtggagtatgtaatag

SEQ ID NO: 7 - DNA, 38 bp, Artificial Sequence
  primer oligonucleotide
taatacgactcactataggggattggagtctgagaatt

SEQ ID NO: 8 - DNA, 43 bp, Artificial Sequence
  primer oligonucleotide
taatacgactcactatagggaaggtgatgctacatacggaaag

SEQ ID NO: 9 - DNA, 38 bp, Artificial Sequence
  primer oligonucleotide
taatacgactcactatagggttgtttgtctgccgtgat

SEQ ID NO: 10 - DNA, 370 bp, Artificial Sequence
  partial coding region
aagtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaa

SEQ ID NO: 11 - DNA, 503 bp, Artificial Sequence
  partial coding region
caccatgggctccagcggcgccctgctgttccacggcaagatcccctacgtggtggagatggagggcaatgtggatggccacaccttcagcatccgcggcaagggctacggcgatgccagcgtgggcaaggtggatgcccagttcatctgcaccaccggcgatgtgcccgtgccctggagcaccctggtgaccaccctgacctacggcgcccagtgcttcgccaagtacggccccgagctgaaggatttctacaagagctgcatgcccgatggctacgtgcaggagcgcaccatcaccttcgagggcgatggcaatttcaagacccgcgccgaggtgaccttcgagaatggcagcgtgtacaatcgcgtgaagctgaatggccagggcttcaagaaggatggccacgtgctgggcaagaatctggagttcaatttcaccccccactgcctgtacatctggggcgatcaggccaatcacggcctgaagagcgccttcaagatct

SEQ ID NO: 12 - DNA, 218 bp, Diabrotica virgifera
tagctctgatgacagagcccatcgagtttcaagccaaacagttgcataaagctatcagcggattgggaactgatgaaagtacaatmgtmgaaattttaagtgtmcacaacaacgatgagattataagaatttcccaggcctatgaaggattgtaccaacgmtcattggaatctgatatcaaaggagatacctcaggaacattaaaaaagaattattag

SEQ ID NO: 13 - DNA, 424 bp, Diabrotica virgifera
  misc_feature (393)..(393): n is a, c, g, or t
ttgttacaagctggagaacttctctttgctggaaccgaagagtcagtatttaatgctgtattctgtcaaagaaataaaccacaattgaatttgatattcgacaaatatgaagaaattgttgggcatcccattgaaaaagccattgaaaacgagttttcaggaaatgctaaacaagccatgttacaccttatccagagcgtaagagatcaagttgcatatttggtaaccaggctgcatgattcaatggcaggcgtcggtactgacgatagaactttaatcagaattgttgtttcgagatctgaaatcgatctagaggaaatcaaacaatgctatgaagaaatctacagtaaaaccttggctgataggatagcggatgacacatctggcgactannnaaaagccttattagccgttgttggttaag

SEQ ID NO: 14 - DNA, 397 bp, Diabrotica virgifera
agatgttggctgcatctagagaattacacaagttcttccatgattgcaaggatgtactgagcagaatagtggaaaaacaggtatccatgtctgatgaattgggaagggacgcaggagctgtcaatgcccttcaacgcaaacaccagaacttcctccaagacctacaaacactccaatcgaacgtccaacaaatccaagaagaatcagctaaacttcaagctagctatgccggtgatagagctaaagaaatcaccaacagggagcaggaagtggtagcagcctgggcagccttgcagatcgcttgcgatcagagacacggaaaattgagcgatactggtgatctattcaaattctttaacttggtacgaacgttgatgcagtggatggacgaatggac

SEQ ID NO: 15 - DNA, 490 bp, Diabrotica virgifera
gcagatgaacaccagcgagaaaccaagagatgttagtggtgttgaattgttgatgaacaaccatcagacactcaaggctgagatcgaagccagagaagacaactttacggcttgtatttctttaggaaaggaattgttgagccgtaatcactatgctagtgctgatattaaggataaattggtcgcgttgacgaatcaaaggaatgctgtactacagaggtgggaagaaagatgggagaacttgcaactcatcctcgaggtataccaattcgccagagatgcggccgtcgccgaagcatggttgatcgcacaagaaccttacttgatgagccaagaactaggacacaccattgacgacgttgaaaacttgataaagaaacacgaagcgttcgaaaaatcggcagcggcgcaagaagagagattcagtgctttggagagactgacgacgttcgaattgagagaaataaagaggaaacaagaagctgcccag

SEQ ID NO: 16 - DNA, 330 bp, Diabrotica virgifera
agtgaaatgttagcaaatataacatccaagtttcgtaattgtacttgctcagttagaaaatattctgtagtttcactatcttcaaccgaaaatagaataaatgtagaacctcgcgaacttgcctttcctccaaaatatcaagaacctcgacaagtttggttggagagtttagatacgatagacgacaaaaaattgggtattcttgagctgcatcctgatgtttttgctactaatccaagaatagatattatacatcaaaatgttagatggcaaagtttatatagatatgtaagctatgctcatacaaagtcaagatttgaagtgagaggt

SEQ ID NO: 17 - DNA, 320 bp, Diabrotica virgifera
caaagtcaagatttgaagtgagaggtggaggtcgaaaaccgtggccgcaaaagggattgggacgtgctcgacatggttcaattagaagtccactttggagaggtggaggagttgttcatggaccaaaatctccaacccctcatttttacatgattccattctacacccgtttgctgggtttgactagcgcactttcagtaaaatttgcccaagatgacttgcacgttgtggatagtctagatctgccaactgacgaacaaagttatatagaagagctggtcaaaagccgcttttgggggtccttcttgttttatttgtag

SEQ ID NO: 18 - DNA, 46 bp, Artificial Sequence
  primer oligonucleotide
ttaatacgactcactatagggagagctccaacagtggttccttatc

SEQ ID NO: 19 - DNA, 29 bp, Artificial Sequence
  primer oligonucleotide
ctaataattcttttttaatgttcctgagg

SEQ ID NO: 20 - DNA, 22 bp, Artificial Sequence
  primer oligonucleotide
gctccaacagtggttccttatc

SEQ ID NO: 21 - DNA, 53 bp, Artificial Sequence
  primer oligonucleotide
ttaatacgactcactatagggagactaataattcttttttaatgttcctgagg

SEQ ID NO: 22 - DNA, 48 bp, Artificial Sequence
  primer oligonucleotide
ttaatacgactcactatagggagattgttacaagctggagaacttctc

SEQ ID NO: 23 - DNA, 24 bp, Artificial Sequence
  primer oligonucleotide
cttaaccaacaacggctaataagg

SEQ ID NO: 24 - DNA, 24 bp, Artificial Sequence
  primer oligonucleotide
ttgttacaagctggagaacttctc

SEQ ID NO: 25 - DNA, 48 bp, Artificial Sequence
  primer oligonucleotide
ttaatacgactcactatagggagacttaaccaacaacggctaataagg

SEQ ID NO: 26 - DNA, 47 bp, Artificial Sequence
  primer oligonucleotide
ttaatacgactcactatagggagaagatgttggctgcatctagagaa

SEQ ID NO: 27 - DNA, 22 bp, Artificial Sequence
  primer oligonucleotide
gtccattcgtccatccactgca

SEQ ID NO: 28 - DNA, 23 bp, Artificial Sequence
  primer oligonucleotide
agatgttggctgcatctagagaa

SEQ ID NO: 29 - DNA, 46 bp, Artificial Sequence
  primer oligonucleotide
ttaatacgactcactatagggagagtccattcgtccatccactgca

SEQ ID NO: 30 - DNA, 46 bp, Artificial Sequence
  primer oligonucleotide
ttaatacgactcactatagggagagcagatgaacaccagcgagaaa

SEQ ID NO: 31 - DNA, 22 bp, Artificial Sequence
  primer oligonucleotide
ctgggcagcttcttgtttcctc

SEQ ID NO: 32 - DNA, 22 bp, Artificial Sequence
  primer oligonucleotide
gcagatgaacaccagcgagaaa

SEQ ID NO: 33 - DNA, 46 bp, Artificial Sequence
  primer oligonucleotide
ttaatacgactcactatagggagactgggcagcttcttgtttcctc

SEQ ID NO: 34 - DNA, 51 bp, Artificial Sequence
  primer oligonucleotide
ttaatacgactcactatagggagaagtgaaatgttagcaaatataacatcc

SEQ ID NO: 35 - DNA, 26 bp, Artificial Sequence
  primer oligonucleotide
acctctcacttcaaatcttgactttg

SEQ ID NO: 36 - DNA, 27 bp, Artificial Sequence
  primer oligonucleotide
agtgaaatgttagcaaatataacatcc

SEQ ID NO: 37 - DNA, 50 bp, Artificial Sequence
  primer oligonucleotide
ttaatacgactcactatagggagaacctctcacttcaaatcttgactttg

SEQ ID NO: 38 - DNA, 50 bp, Artificial Sequence
  primer oligonucleotide
ttaatacgactcactatagggagacaaagtcaagatttgaagtgagaggt

SEQ ID NO: 39 - DNA, 25 bp, Artificial Sequence
  primer oligonucleotide
ctacaaataaaacaagaaggacccc

SEQ ID NO: 40 - DNA, 26 bp, Artificial Sequence
  primer oligonucleotide
caaagtcaagatttgaagtgagaggt

SEQ ID NO: 41 - DNA, 49 bp, Artificial Sequence
  primer oligonucleotide
ttaatacgactcactatagggagactacaaataaaacaagaaggacccc

SEQ ID NO: 42 - DNA, 47 bp, Artificial Sequence
  primer oligonucleotide
ttaatacgactcactatagggagacaccatgggctccagcggcgccc

SEQ ID NO: 43 - DNA, 23 bp, Artificial Sequence
  primer oligonucleotide
agatcttgaaggcgctcttcagg

SEQ ID NO: 44 - DNA, 23 bp, Artificial Sequence
  primer oligonucleotide
caccatgggctccagcggcgccc

SEQ ID NO: 45 - DNA, 47 bp, Artificial Sequence
  primer oligonucleotide
ttaatacgactcactatagggagaagatcttgaaggcgctcttcagg

SEQ ID NO: 46 - DNA, 222 bp, Solanum tuberosum
gactagtaccggttgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatccgcgg

SEQ ID NO: 47 - DNA, 22 bp, Artificial Sequence
  primer oligonucleotide
ttttttttttttttttttttvn

SEQ ID NO: 48 - DNA, 19 bp, Artificial Sequence
  primer oligonucleotide
tgtgatgttggtggcgtat

SEQ ID NO: 49 - DNA, 24 bp, Artificial Sequence
  primer oligonucleotide
tgttaaataaaaccccaaagatcg

SEQ ID NO: 50 - DNA, 20 bp, Artificial Sequence
  primer oligonucleotide
gagggtaatgccaactggtt

SEQ ID NO: 51 - DNA, 24 bp, Artificial Sequence
  primer oligonucleotide
gcaatgtaaccgagtgtctctcaa

SEQ ID NO: 52 - DNA, 32 bp, Artificial Sequence
  probe oligonucleotide
tttttggcttagagttgatggtgtactgatga

SEQ ID NO: 53 - DNA, 151 bp, Escherichia coli
gaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaag

SEQ ID NO: 54 - DNA, 69 bp, Artificial Sequence
  partial coding region
tgttcggttccctctaccaagcacagaaccgtcgcttcagcaacacctcagtcaaggtgatggatgttg

SEQ ID NO: 55 - DNA, 25 bp, Artificial Sequence
  primer oligonucleotide
gtatgtttctgcttctacctttgat

SEQ ID NO: 56 - DNA, 29 bp, Artificial Sequence
  primer oligonucleotide
ccatgttttggtcatatattagaaaagtt

SEQ ID NO: 57 - DNA, 34 bp, Artificial Sequence
  probe oligonucleotide
agtaatatagtatttcaagtatttttttcaaaat

SEQ ID NO: 58 - DNA, 20 bp, Artificial Sequence
  primer oligonucleotide
tgttcggttccctctaccaa

SEQ ID NO: 59 - DNA, 22 bp, Artificial Sequence
  primer oligonucleotide
caacatccatcaccttgactga

SEQ ID NO: 60 - DNA, 24 bp, Artificial Sequence
  probe oligonucleotide
cacagaaccgtcgcttcagcaaca

SEQ ID NO: 61 - DNA, 18 bp, Artificial Sequence
  primer oligonucleotide
tggcggacgacgacttgt

SEQ ID NO: 62 - DNA, 19 bp, Artificial Sequence
  primer oligonucleotide
aaagtttggaggctgccgt

SEQ ID NO: 63 - DNA, 26 bp, Artificial Sequence
  probe oligonucleotide
cgagcagaccgccgtgtacttctacc

SEQ ID NO: 64 - DNA, 19 bp, Artificial Sequence
  primer oligonucleotide
cttagctggataacgccac

SEQ ID NO: 65 - DNA, 19 bp, Artificial Sequence
  primer oligonucleotide
gaccgtaaggcttgatgaa

SEQ ID NO: 66 - DNA, 21 bp, Artificial Sequence
  probe oligonucleotide
cgagattctccgcgctgtaga

SEQ ID NO: 67 - RNA, 1617 nt, Diabrotica virgifera
aaaucaaguuacuuagcaaaacgaaagcuacuugcuuuguaguuuaauuuuuauguguauuaaauagcucacagaaacuguguauuagugauuggauauugauaguuuauauuaucaaacauuuuguuccgugugucuauacauaaaaacauguuuuuugguaaaugucuggauaccucaaauaauaauacguuuuucaauuuccacacaucccgagagaugaaggcagacgagaagccaccgauuaagccucaccaaagcaccuucggguacccugcguuuccaaacccagcgauguuuaccccaucacaaauccuaauggcuagucaacuaauggcagcauccggcuugaccauaucuggaaacccugccuucuuccauccagguuugaugccccuaccaugggcagcaaacucaccuccuuccaauucuccacccucaccaccgaccagcaacgaaccgcuguccccugcgaugaguacaagaaaacuuagucauaauaacaauaauguuguuaguaguaguacgacgacagaaauuaggaaaagaaaguauaaguuagaagaagaagccacgaguuuaccgcugccauccccuacgucuaguguaucgccuccuucggggucugauauuaaagauccuaauagggauaagcaguucaccuguggaguauguaauagaucauuuggcuacaaacacguccugcaaaaucacgaaaggacacauaccggagagaaaccuuucgaauguccagagugccacaaaagguucaccagagaccaucaccuuaagacccacaugagacuucauacaggagaacgcccuuaucauugcgaacacugugacagacaauucguccaaguugccaaccuucgaagacauuugagggugcauacaggagaacgaccuuacucuugugaacacuguacugccaaauucucagacuccaaucagcucaaggcucacauacuuauccauaccaaugaaaaaccauuuucgugugaaaggugucacagcagguucagaaggaggcaucacuuguugcaccauaaaugcggcaugccugauaaagaaaugacuccugauacagaugaagaggaguuaaagaggaggagagugcuacauucucuuucuacgccaauuauuacgcuuccucuccaaguagcucuaccagaacaaaccgagcccgaagaccucaguaugucgacggggaugcacucaaacaauucgggaagcagcggcuccccgaucguaaguccuuguuuaaaagaagaagaggacgacaucgaaacaacugcgauauuucuacaaagaaggccaaugaguagauccuagucuaguuauuuucgugagauauacggguauguuaaagucaaauuauaauuauccaaaauauauacauagucccuuaucacagcgucacuuccagaggaauuguuuuggggaauaaaaaagucuaaaaggaaagaaguaacuguuacacacuugcaaauaauuucuaauacauauauuauauuuacuugcacuacagguauuguguugaauuuuuuauagaacaauagauuucaaauaacaaguaauaaaauacucauuuauauuacaguggaaccccgauaa

SEQ ID NO: 68 - RNA, 1640 nt, Diabrotica virgifera
cugacagucaauagguguaguuauacgacauuuagauagauuugauaaacauuuacgacgacagcacgaauauuuuuuauuuucuuuuguacauuuuuguauuuucguggugcgugaugaagugacaauggacuggguuugugcgugguuuugauuuuguuuuugcuauuucgauuuuuguuuuugaaaugccgcuucuacaagaaaauacuccaaaaagagauuuccacacaucccgagagaugaaggcagacgagaagccaccgauuaagccucaccaaagcaccuucggguacccugcguuuccaaacccagcgauguuuaccccaucacaaauccuaauggcuagucaacuaauggcagcauccggcuugaccauaucuggaaacccugccuucuuccauccagguuugaugccccuaccaugggcagcaaacucaccuccuuccaauucuccacccucaccaccgaccagcaacgaaccgcuguccccugcgaugaguacaagaaaacuuagucauaauaacaauaauguuguuaguaguaguacgacgacagaaauuaggaaaagaaaguauaaguuagaagaagaagccaccaguuuaccgcugccauccccuacgucuaguguaucgccuccuucggggucugauauuaaagauccuaauagggauaagcaguucaccuguggaguauguaauagaucauuuggcuacaaacacguccugcaaaaucacgaaaggacacauaccggagagaaaccuuucgaauguccagagugccacaaaagguucaccagagaccaucaccuuaagacccacaugagacuucauacaggagaacgcccuuaucauugcgaacacugugacagacaauucguccaaguugccaaccuucgaagacauuugagggugcauacaggagaacgaccuuacucuugugaacacuguacugccaaauucucagacuccaaucagcucaaggcucacauacuuauccauaccaaugaaaaaccauuuucgugugaaaggugucacagcagguucagaaggaggcaucacuuguugcaccauaaaugcggcaugccugauaaagaaaugacuccugauacagaugaagaggaguuaaagaggaggagagugcuacauucucuuucuacgccaauuauuacgcuuccucuccaaguagcucuaccagaacaaaccgagcccgaagaccucaguaugucgacggggaugcacucaaacaauucgggaagcagcggcuccccgaucguaaguccuuguuuaaaagaagaagaggacgacaucgaaacaacugcgauauuucuacaaagaaggccaaugaguagauccuagucuaguuauuuucgugagauauacggguauguuaaagucaaauuauaauuauccaaaauauauacauagucccuuaucacagcgucacuuccagaggaauuguuuuggggaauaaaaaagucuaaaaggaaagaaguaacuguuacacacuugcaaauaauuucuaauacauauauuauauuuacuugcacuacaggucuuguguugaauuuuuuauagaacaauagauuucaaauaacaaguaauaaaauacucauuuauauuacaguggaaccccgauaa

SEQ ID NO: 69 - RNA, 291 nt, Diabrotica virgifera
ccuguggaguauguaauagaucauuuggcuacaaacacguccugcaaaaucacgaaaggacacauaccggagagaaaccuuucgaauguccagagugccacaaaagguucaccagagaccaucaccuuaagacccacaugagacuucauacaggagaacgcccuuaucauugcgaacacugugacagacaauucguccaaguugccaaccuucgaagacauuugagggugcauacaggagaacgaccuuacucuugugaacacuguacugccaaauucucagacuccaauc

(19) United States
(12) Patent Application Publication
(10) Pub. No.: US 2016/0208253 A1
(43) Pub. Date: Jul. 21, 2016

(54) PARENTAL RNAI SUPPRESSION OF KRUPPEL GENE TO CONTROL COLEOPTERAN PESTS

(71) Applicants: The Board of Regents of the University of Nebraska, Lincoln, NE (US); Dow AgroSciences LLC, Zionsville, IN (US)

(72) Inventors: Blair D. Siegfried, Lincoln, NE (US); Kenneth E. Narva, Zionsville, IN (US); Kanika Arora, Indianapolis, IN (US); Sarah E. Worden, Indianapolis, IN (US); Chitvan Khajuria, Chesterfield, MO (US); Elane Fishilevich, Indianapolis, IN (US); Nicholas P. Storer, Kensington, MD (US); Meghan Frey, Greenwood, IN (US); Ronda L. Hamm, Carmel, IN (US); Ana Velez, Lincoln, NE (US)

(21) Appl. No.: 14/971,479

(22) Filed: Dec. 16, 2015

Related U.S. Application Data
(60) Provisional application No. 62/092,781, filed on Dec. 16, 2014.

(51) Int. Cl. C12N 15/113; A01N 57/16; C12N 15/82

(52) U.S. Cl. CPC C12N 15/113; C12N 15/8218; C12N 15/8286; A01N 57/16; C12N 2310/14

(57) ABSTRACT

This disclosure concerns nucleic acid molecules and methods of use thereof for control of coleopteran pests through RNA interference-mediated inhibition of target coding and transcribed non-coding sequences in coleopteran pests. The disclosure also concerns methods for making transgenic plants that express nucleic acid molecules useful for the control of coleopteran pests, and the plant cells and plants obtained thereby.

### PRIORITY CLAIM

This application claims the benefit of the filing date of U.S. Provisional Patent Application Ser. No. 62/092,781, filed Dec. 16, 2014, for “PARENTAL RNAI SUPPRESSION OF KRUPPEL GENE TO CONTROL COLEOPTERAN PESTS”, which is incorporated herein in its entirety.

### FIELD OF THE DISCLOSURE

The present invention relates generally to genetic control of plant damage caused by coleopteran pests. In particular embodiments, the present disclosure relates to identification of target coding and non-coding polynucleotides, and the use of recombinant DNA technologies for post-transcriptionally repressing or inhibiting expression of target coding and non-coding polynucleotides in the cells of a coleopteran pest to provide a plant protective effect.

### BACKGROUND

The western corn rootworm (WCR), Diabrotica virgifera virgifera LeConte, is one of the most devastating corn rootworm species in North America and is a particular concern in corn-growing areas of the Midwestern United States. The northern corn rootworm (NCR), Diabrotica barberi Smith and Lawrence, is a closely-related species that co-inhabits much of the same range as WCR. There are several other related subspecies of Diabrotica that are significant pests in the Americas: the Mexican corn rootworm (MCR), D. virgifera zeae Krysan and Smith; the southern corn rootworm (SCR), D. undecimpunctata howardi Barber; D. balteata LeConte; D. undecimpunctata tenella; D. speciosa Germar; and D. u. undecimpunctata Mannerheim. The United States Department of Agriculture has estimated that corn rootworms cause $1 billion in lost revenue each year, including $800 million in yield loss and $200 million in treatment costs.

Both WCR and NCR are deposited in the soil as eggs during the summer. The insects remain in the egg stage throughout the winter. The eggs are oblong, white, and less than 0.004 inches in length. The larvae hatch in late May or early June, with the precise timing of egg hatching varying from year to year due to temperature differences and location. The newly hatched larvae are white worms that are less than 0.125 inches in length. Once hatched, the larvae begin to feed on corn roots. Corn rootworms go through three larval instars. After feeding for several weeks, the larvae molt into the pupal stage. They pupate in the soil, and then they emerge from the soil as adults in July and August. Adult rootworms are about 0.25 inches in length.

Corn rootworm larvae complete development on corn and several other species of grasses. Larvae reared on yellow foxtail emerge later and have a smaller head capsule size as adults compared to larvae reared on corn. Ellsbury et al. (2005) Environ. Entomol. 34:627-34. WCR adults feed on corn silk, pollen, and exposed ear tips. Adults will quickly shift to preferred tissues such as silks and pollen when they become available. NCR adults also feed on reproductive tissues of the corn plant. WCR females typically mate once. Branson et al. (1977) Ann. Entom. Soc. America 70(4):506-8.

Most of the rootworm damage in corn is caused by larval feeding. Newly hatched rootworms initially feed on fine corn root hairs and burrow into root tips. As the larvae grow larger, they feed on and burrow into primary roots. When corn rootworms are abundant, larval feeding often results in the pruning of roots all the way to the base of the corn stalk. Severe root injury interferes with the roots' ability to transport water and nutrients into the plant, reduces plant growth, and results in reduced grain production, thereby often drastically reducing overall yield. Severe root injury also often results in lodging of corn plants, which makes harvest more difficult and further decreases yield. Furthermore, feeding by adults on the corn reproductive tissues can result in pruning of silks at the ear tip. If this “silk clipping” is severe enough during pollen shed, pollination may be disrupted.

Control of corn rootworms may be attempted by crop rotation, chemical insecticides, biopesticides (e.g., the spore-forming gram-positive bacterium, Bacillus thuringiensis), transgenic plants that express Bt toxins, or a combination thereof. Crop rotation suffers from the disadvantage of placing restrictions upon the use of farmland. Moreover, oviposition of some rootworm species may occur in crop fields other than corn or extended diapause results in egg hatching over multiple years, thereby mitigating the effectiveness of crop rotation practiced with corn and other crops.

Chemical insecticides are the most heavily relied upon strategy for achieving corn rootworm control. Chemical insecticide use, though, is an imperfect corn rootworm control strategy; over $1 billion may be lost in the United States each year due to corn rootworm infestations when the costs of the chemical insecticides are added to the costs of yield loss from the rootworm injury that may occur despite the use of the insecticides. High populations of larvae, heavy rains, and improper application of the insecticide(s) may all result in inadequate corn rootworm control. Furthermore, the continual use of insecticides may select for insecticide-resistant rootworm strains, as well as raise significant environmental concerns due to their lack of specificity.

RNA interference (RNAi) is a process utilizing endogenous cellular pathways, whereby an interfering RNA (iRNA) molecule (e.g., a double stranded RNA (dsRNA) molecule) that is specific for all, or any portion of adequate size, of a target gene results in the degradation of the mRNA encoded thereby. In recent years, RNAi has been used to perform gene “knockdown” in a number of species and experimental systems; for example, Caenorhabditis elegans, plants, insect embryos, and cells in tissue culture. See, e.g., Fire et al. (1998) Nature 391:806-11; Martinez et al. (2002) Cell 110:563-74; McManus and Sharp (2002) Nature Rev. Genetics 3:737-47.

RNAi accomplishes degradation of mRNA through an endogenous pathway including the DICER protein complex. DICER cleaves long dsRNA molecules into short fragments of approximately 20 nucleotides, termed small interfering RNA (siRNA). The siRNA is unwound into two single-stranded RNAs: the passenger strand and the guide strand. The passenger strand is degraded, and the guide strand is incorporated into the RNA-induced silencing complex (RISC). Micro ribonucleic acids (miRNAs) are structurally very similar molecules that are cleaved from precursor molecules containing a polynucleotide “loop” connecting the hybridized passenger and guide strands, and they may be similarly incorporated into RISC. Post-transcriptional gene silencing occurs when the guide strand binds specifically to a complementary mRNA molecule and induces cleavage by Argonaute, the catalytic component of the RISC complex. This process is known to spread systemically throughout some eukaryotic organisms despite initially limited concentrations of siRNA and/or miRNA, such as plants, nematodes, and some insects.

Only transcripts complementary to the siRNA and/or miRNA are cleaved and degraded, and thus the knock-down of mRNA expression is sequence-specific. In plants, several functional groups of DICER genes exist. The gene silencing effect of RNAi persists for days and, under experimental conditions, can lead to a decline in abundance of the targeted transcript of 90% or more, with consequent reduction in levels of the corresponding protein. In insects, there are at least two DICER genes, where DICER1 facilitates miRNA-directed degradation by Argonaute1. Lee et al. (2004) Cell 117(1):69-81. DICER2 facilitates siRNA-directed degradation by Argonaute2.

U.S. Pat. No. 7,612,194 and U.S. Patent Publication Nos. 2007/0050860, 2010/0192265, and 2011/0154545 disclose a library of 9112 expressed sequence tag (EST) sequences isolated from D. v. virgifera LeConte pupae. It is suggested in U.S. Pat. No. 7,612,194 and U.S. Patent Publication No. 2007/0050860 to operably link to a promoter a nucleic acid molecule that is complementary to one of several particular partial sequences of D. v. virgifera vacuolar-type H⁺-ATPase (V-ATPase) disclosed therein for the expression of anti-sense RNA in plant cells. U.S. Patent Publication No. 2010/0192265 suggests operably linking a promoter to a nucleic acid molecule that is complementary to a particular partial sequence of a D. v. virgifera gene of unknown and undisclosed function (the partial sequence is stated to be 58% identical to C56C10.3 gene product in C. elegans) for the expression of anti-sense RNA in plant cells. U.S. Patent Publication No. 2011/0154545 suggests operably linking a promoter to a nucleic acid molecule that is complementary to two particular partial sequences of D. v. virgifera coatomer beta subunit genes for the expression of anti-sense RNA in plant cells. Further, U.S. Pat. No. 7,943,819 discloses a library of 906 expressed sequence tag (EST) sequences isolated from D. v. virgifera LeConte larvae, pupae, and dissected midguts, and suggests operably linking a promoter to a nucleic acid molecule that is complementary to a particular partial sequence of a D. v. virgifera charged multivesicular body protein 4b gene for the expression of double-stranded RNA in plant cells.

No further suggestion is provided in U.S. Pat. No. 7,612,194, and U.S. Patent Publication Nos. 2007/0050860, 2010/0192265, and 2011/0154545 to use any particular sequence of the more than nine thousand sequences listed therein for RNA interference, other than the several particular partial sequences of V-ATPase and the particular partial sequences of genes of unknown function. Furthermore, none of U.S. Pat. No. 7,612,194, and U.S. Patent Publication Nos. 2007/0050860 and 2010/0192265, and 2011/0154545 provides any guidance as to which other of the over nine thousand sequences provided would be lethal, or even otherwise useful, in species of corn rootworm when used as dsRNA or siRNA. U.S. Pat. No. 7,943,819 provides no suggestion to use any particular sequence of the more than nine hundred sequences listed therein for RNA interference, other than the particular partial sequence of a charged multivesicular body protein 4b gene. Furthermore, U.S. Pat. No. 7,943,819 provides no guidance as to which other of the over nine hundred sequences provided would be lethal, or even otherwise useful, in species of corn rootworm when used as dsRNA or siRNA. U.S. Patent Application Publication No. U.S. 2013/040173 and PCT Application Publication No. WO 2013/169923 describe the use of a sequence derived from a Diabrotica virgifera Snf7 gene for RNA interference in maize. (Also disclosed in Bolognesi et al. (2012) PLOS ONE 7(10): e47534. doi:10.1371/journal.pone.0047534).

The overwhelming majority of sequences complementary to corn rootworm DNAs (such as the foregoing) do not provide a plant protective effect from species of corn rootworm when used as dsRNA or siRNA. For example, Baum et al. (2007) Nature Biotechnology 25:1322-1326, described the effects of inhibiting several WCR gene targets by RNAi. These authors reported that 8 of the 26 target genes they tested were not able to provide experimentally significant coleopteran pest mortality at a very high iRNA (e.g., dsRNA) concentration of more than 520 ng/cm².

The authors of U.S. Pat. No. 7,612,194 and U.S. Patent Publication No. 2007/0050860 made the first report of in planta RNAi in corn plants targeting the western corn rootworm. Baum et al. (2007) Nat. Biotechnol. 25(11):1322-6. These authors describe a high-throughput in vivo dietary RNAi system to screen potential target genes for developing transgenic RNAi maize. Of an initial gene pool of 290 targets, only 14 exhibited larval control potential. One of the most effective double-stranded RNAs (dsRNA) targeted a gene encoding vacuolar ATPase subunit A (V-ATPase), resulting in a rapid suppression of corresponding endogenous mRNA and triggering a specific RNAi response with low concentrations of dsRNA. Thus, these authors documented for the first time the potential for in planta RNAi as a possible pest management tool, while simultaneously demonstrating that effective targets could not be accurately identified a priori, even from a relatively small set of candidate genes.

Another potential application of RNAi for insect control involves parental RNAi (pRNAi). First described in Caenorhabditis elegans, pRNAi was identified by injection of dsRNA into the body cavity (or application of dsRNA via ingestion), causing gene inactivity in offspring embryos. Fire et al. (1998), supra; Timmons and Fire (1998) Nature 395(6705):854. A similar process was described in the model coleopteran, Tribolium castaneum, whereby female pupae injected with dsRNA corresponding to three unique genes that control segmentation during embryonic development resulted in knock down of zygotic genes in offspring embryos. Bucher et al. (2002) Curr. Biol. 12(3):R85-6. Nearly all of the offspring larvae in this study displayed gene-specific phenotypes one week after injection. Although injection of dsRNA for functional genomics studies has been successful in a variety of insects, uptake of dsRNA from the gut environment through oral exposure to dsRNA and subsequent down-regulation of essential genes is required in order for RNAi to be effective as a pest management tool. Auer and Frederick (2009) Trends Biotechnol. 27(11):644-51.

Parental RNAi has been used to describe the function of embryonic genes in a number of insect species, including the springtail, Orchesella cincta (Konopova and Akam (2014) Evodevo 5(1):2); the brown plant hopper, Nilaparvata lugens; the sawfly, Athalia rosae (Yoshiyama et al. (2013) J. Insect Physiol. 59(4):400-7); the German cockroach, Blattella germanica (Piulachs et al. (2010) Insect Biochem. Mol. Biol. 40:468-75); and the pea aphid, Acyrthosiphon pisum (Mao et al. (2013) Arch Insect Biochem Physiol 84(4):209-21). The pRNAi response in all these instances was achieved by injection of dsRNA into the hemocoel of the parental female.

### SUMMARY OF THE DISCLOSURE

Disclosed herein are nucleic acid molecules (e.g., target genes, DNAs, dsRNAs, siRNAs, shRNAs, miRNAs, and hpRNAs), and methods of use thereof, for the control of coleopteran pests, including, for example, D. v. virgifera LeConte (western corn rootworm, “WCR”); D. barberi Smith and Lawrence (northern corn rootworm, “NCR”); D. u. howardi Barber (southern corn rootworm, “SCR”); D. v. zeae Krysan and Smith (Mexican corn rootworm, “MCR”); D. balteata LeConte; D. u. tenella; D. speciosa Germar; and D. u. undecimpunctata Mannerheim. In particular examples, exemplary nucleic acid molecules are disclosed that may be homologous to at least a portion of one or more native nucleic acids in a coleopteran pest. In some embodiments, coleopteran pests are controlled by reducing the capacity of an existing generation to produce a subsequent generation of the pest. In certain examples, delivery of the nucleic acid molecules to coleopteran pests does not result in significant mortality to the pests, but reduces the number of viable progeny produced therefrom.

In these and further examples, the native nucleic acid may be a target gene, the product of which may be, for example and without limitation: involved in a metabolic process; involved in a reproductive process; and/or involved in embryonic and/or larval development. In some examples, post-transcriptional inhibition of the expression of a target gene by a nucleic acid molecule comprising a polynucleotide homologous thereto may result in reduced viability, growth, and/or reproduction of the coleopteran pest. In specific examples, a kruppel gene is selected as a target gene for post-transcriptional silencing. In particular examples, a target gene useful for post-transcriptional inhibition is the novel gene referred to herein as Diabrotica kruppel (SEQ ID NO:1 and SEQ ID NO:2). An isolated nucleic acid molecule comprising the polynucleotide of SEQ ID NO:1; the complement of SEQ ID NO:1; SEQ ID NO:2; the complement of SEQ ID NO:2; and/or fragments of either of the foregoing (e.g., SEQ ID NO:4) is therefore disclosed herein.

Also disclosed are nucleic acid molecules comprising polynucleotides that encode polypeptides that are at least about 85% identical to an amino acid sequence within a target gene product (for example, the product of a kruppel gene). For example, a nucleic acid molecule may comprise a polynucleotide encoding a polypeptide that is at least 85% identical to SEQ ID NO:3 (Diabrotica KRUPPEL); and/or an amino acid sequence within a product of Diabrotica kruppel. Further disclosed are nucleic acid molecules comprising a polynucleotide that is the reverse complement of a polynucleotide that encodes a polypeptide at least 85% identical to an amino acid sequence within a target gene product.

Further disclosed are cDNA polynucleotides that may be used for the production of iRNA (e.g., dsRNA, siRNA, shRNA, miRNA, and hpRNA) molecules that are complementary to all or part of a coleopteran pest target gene, for example, a kruppel gene. In particular embodiments, dsRNAs, siRNAs, shRNAs, miRNAs, and/or hpRNAs may be produced in vitro, or in vivo by a genetically-modified organism, such as a plant or bacterium. In particular examples, cDNA molecules are disclosed that may be used to produce iRNA molecules that are complementary to all or part of mRNA transcribed from Diabrotica kruppel (SEQ ID NO:1 and SEQ ID NO:2).

Additionally disclosed are means for inhibiting expression of an essential gene in a coleopteran pest, and means for protecting a plant from a coleopteran pest. A means for inhibiting expression of an essential gene in a coleopteran pest is a single- or double-stranded RNA molecule consisting of a polynucleotide selected from the group consisting of SEQ ID NOs:69-71; and the complements thereof. Functional equivalents of means for inhibiting expression of an essential gene in a coleopteran pest include single- or double-stranded RNA molecules that are substantially homologous to all or part of mRNA transcribed from a WCR gene comprising SEQ ID NO:1 or SEQ ID NO:2. A means for protecting a plant from a coleopteran pest is a DNA molecule comprising a polynucleotide encoding a means for inhibiting expression of an essential gene in a coleopteran pest operably linked to a promoter, wherein the DNA molecule is capable of being integrated into the genome of a maize plant.

Disclosed are methods for controlling a population of a coleopteran pest, comprising providing to a coleopteran pest an iRNA (e.g., dsRNA, siRNA, shRNA, miRNA, and hpRNA) molecule that functions upon being taken up by the pest to inhibit a biological function within the pest, wherein the iRNA molecule comprises all or part of (e.g., at least 15 contiguous nucleotides of) a polynucleotide selected from the group consisting of: SEQ ID NO:1; the complement of SEQ ID NO:1; SEQ ID NO:2; the complement of SEQ ID NO:2; a native coding polynucleotide of a Diabrotica organism (e.g., WCR) comprising all or part of SEQ ID NO:1 and/or SEQ ID NO:2; the complement of a native coding polynucleotide of a Diabrotica organism comprising all or part of SEQ ID NO:1 and/or SEQ ID NO:2; a native non-coding polynucleotide of a Diabrotica organism that is transcribed into a native RNA molecule comprising all or part of SEQ ID NO:1 and/or SEQ ID NO:2; and the complement of a native non-coding polynucleotide of a Diabrotica organism that is transcribed into a native RNA molecule comprising all or part of SEQ ID NO:1 and/or SEQ ID NO:2.

In particular examples, methods are disclosed for controlling a population of a coleopteran pest, comprising providing to a coleopteran pest an iRNA (e.g., dsRNA, siRNA, shRNA, miRNA, and hpRNA) molecule that functions upon being taken up by the pest to inhibit a biological function within the pest, wherein the iRNA molecule comprises a polynucleotide selected from the group consisting of: all or part of SEQ ID NO:67; the complement of all or part of SEQ ID NO:67; all or part of SEQ ID NO:68; the complement of all or part of SEQ ID NO:68; SEQ ID NO:69; and the complement of SEQ ID NO:69; a polynucleotide that hybridizes to a native coding polynucleotide of a Diabrotica organism (e.g., WCR) comprising all or part of either of SEQ ID NOs:1 and 2; and the complement of a polynucleotide that hybridizes to a native coding polynucleotide of a Diabrotica organism (e.g., WCR) comprising all or part of either of SEQ ID NOs:1 and 2.

Also disclosed herein are methods wherein dsRNAs, siRNAs, shRNAs, miRNAs, and/or hpRNAs may be provided to a coleopteran pest in a diet-based assay, or in genetically-modified plant cells expressing the dsRNAs, siRNAs, shRNAs, miRNAs, and/or hpRNAs. In these and further examples, the dsRNAs, siRNAs, shRNAs, miRNAs, and/or hpRNAs may be ingested by a coleopteran pest. Ingestion of dsRNAs, siRNA, shRNAs, miRNAs, and/or hpRNAs of the invention may then result in RNAi in the pest, which in turn may result in silencing of a gene essential for a metabolic process; a reproductive process; and/or a larval development process. Thus, methods are disclosed wherein nucleic acid molecules comprising exemplary polynucleotide(s) useful for parental control of coleopteran pests are provided to a coleopteran pest. In particular examples, the coleopteran pest controlled by use of nucleic acid molecules of the invention may be WCR, NCR or SCR. In some examples, delivery of the nucleic acid molecules to coleopteran pests does not result in significant mortality to the pests, but reduces the number of viable progeny produced therefrom. In some examples, delivery of the nucleic acid molecules to a coleopteran pest results in significant mortality to the pests, and also reduces the number of viable progeny produced therefrom.

The foregoing and other features will become more apparent from the following Detailed Description of several embodiments, which proceeds with reference to the accompanying Figures.

### BRIEF DESCRIPTION OF THE FIGURES

FIG. 1A includes a depiction of the strategy used to generate dsRNA from a single transcription template with a single pair of primers, and FIG. 1B includes a depiction of the strategy used to generate dsRNA from two transcription templates.

FIG. 2 includes a depiction of the domain organization of the Drosophila melanogaster (DME) and D. v. virgifera (WCR) KRUPPEL protein sequences. D. melanogaster and D. v. virgifera KRUPPEL proteins contain 4 C2H2-type zinc finger domains; annotated using SMART database.

FIG. 3A includes a summary of data showing effects of particular dsRNAs on WCR egg production and viability. Depicted are the number of eggs oviposited per adult WCR female, and FIG. 3B shows the percent of eggs that hatched. Data are mean plus/minus the SEM. Bars with * are significantly different (P<0.1) and ** are significantly different (P<0.05) from the water controls.

FIG. 4A and FIG. 4B include representative photographs of WCR eggs dissected to examine embryonic development under different experimental conditions. Eggs that were oviposited by females treated with water and GFP dsRNA (FIG. 4A) show normal development. Eggs oviposited by females treated with kruppel dsRNA (FIG. 4B) show incomplete embryonic development and malformed larvae.

FIG. 5A includes a summary of data showing the relative expression of kruppel in adult WCR females exposed to dsRNA in a treated artificial diet, relative to GFP and water controls. Also shown is the relative expression of kruppel in eggs collected from adult females exposed to dsRNA in a treated artificial diet, relative to GFP and water controls (FIG. 5B), and in larvae exposed to dsRNA in a treated artificial diet, relative to GFP and water controls (FIG. 5C). Error bars represent standard errors of the mean. Bars with ** are significantly different (P<0.05) from water controls.

FIG. 6 includes a summary of modeling data showing the effect of relative magnitude of a pRNAi effect on female WCR adults emerging from a “refuge patch” (i.e., that did not express insecticidal iRNAs or recombinant proteins in a transgenic crop) on the rate of increase in allele frequencies for resistance to an insecticidal protein (R) and RNAi (Y) when non-refuge plants express the insecticidal protein and parental active iRNA.

FIG. 7 includes a summary of modeling data showing the effect of relative magnitude of a pRNAi effect on female WCR adults emerging from a “refuge patch” (i.e., that did not express insecticidal iRNAs or recombinant proteins in a transgenic crop of plants comprising corn rootworm larval-active interfering dsRNA in combination with the corn rootworm-active insecticidal protein in the transgenic crop) on the rate of increase in allele frequencies for resistance to an insecticidal protein (R) and RNAi (Y) when non-refuge plants express the insecticidal protein and both larval active and parental active iRNA molecules.

### BRIEF DESCRIPTION OF SEQUENCE LISTING

The nucleic acid sequences identified in the accompanying sequence listing are shown using standard letter abbreviations for nucleotide bases, as defined in 37 C.F.R. §1.822. The nucleic acid and amino acid sequences listed define molecules (i.e polynucleotides and polypeptides, respectively) having the nucleotide and amino acid monomers arranged in the manner described. The nucleic acid and amino acid sequences listed also each define a genus of polynucleotides or polypeptides that comprise the nucleotide and amino acid monomers arranged in the manner described. In view of the redundancy of the genetic code, it will be understood that a nucleotide sequence including a coding sequence also describes the genus of polynucleotides encoding the same polypeptide as a polynucleotide consisting of the reference sequence. It will further be understood that an amino acid sequence describes the genus of polynucleotide ORFs encoding that polypeptide.

Only one strand of each nucleic acid sequence is shown, but the complementary strand is understood as included by any reference to the displayed strand. As the complement and reverse complement of a primary nucleic acid sequence are necessarily disclosed by the primary sequence, the complementary sequence and reverse complementary sequence of a nucleic acid sequence are included by any reference to the nucleic acid sequence, unless it is explicitly stated to be otherwise (or it is clear to be otherwise from the context in which the sequence appears). Furthermore, as it is understood in the art that the nucleotide sequence of an RNA strand is determined by the sequence of the DNA from which it was transcribed (but for the substitution of uracil (U) nucleobases for thymine (T)), an RNA sequence is included by any reference to the DNA sequence encoding it. In the accompanying sequence listing:

SEQ ID NO:1 shows a contig comprising an exemplary Diabrotica kruppel DNA:

AAATCAAGTTACTTAGCAAAACGAAAGCTACTTGCTTTGTAGTTTAATTT
TTATGTGTATTAAATAGCTCACAGAAACTGTGTATTAGTGATTGGATATT
GATAGTTTATATTATCAAACATTTTGTTCCGTGTGTCTATACATAAAAAC
ATGTTTTTTGGTAAATGTCTGGATACCTCAAATAATAATACGTTTTTCAA
TTTCCACACATCCCGAGAGATGAAGGCAGACGAGAAGCCACCGATTAAGC
CTCACCAAAGCACCTTCGGGTACCCTGCGTTTCCAAACCCAGCGATGTTT
ACCCCATCACAAATCCTAATGGCTAGTCAACTAATGGCAGCATCCGGCTT
GACCATATCTGGAAACCCTGCCTTCTTCCATCCAGGTTTGATGCCCCTAC
CATGGGCAGCAAACTCACCTCCTTCCAATTCTCCACCCTCACCACCGACC
AGCAACGAACCGCTGTCCCCTGCGATGAGTACAAGAAAACTTAGTCATAA
TAACAATAATGTTGTTAGTAGTAGTACGACGACAGAAATTAGGAAAAGAA
AGTATAAGTTAGAAGAAGAAGCCACGAGTTTACCGCTGCCATCCCCTACG
TCTAGTGTATCGCCTCCTTCGGGGTCTGATATTAAAGATCCTAATAGGGA
TAAGCAGTTCACCTGTGGAGTATGTAATAGATCATTTGGCTACAAACACG
TCCTGCAAAATCACGAAAGGACACATACCGGAGAGAAACCTTTCGAATGT
CCAGAGTGCCACAAAAGGTTCACCAGAGACCATCACCTTAAGACCCACAT
GAGACTTCATACAGGAGAACGCCCTTATCATTGCGAACACTGTGACAGAC
AATTCGTCCAAGTTGCCAACCTTCGAAGACATTTGAGGGTGCATACAGGA
GAACGACCTTACTCTTGTGAACACTGTACTGCCAAATTCTCAGACTCCAA
TCAGCTCAAGGCTCACATACTTATCCATACCAATGAAAAACCATTTTCGT
GTGAAAGGTGTCACAGCAGGTTCAGAAGGAGGCATCACTTGTTGCACCAT
AAATGCGGCATGCCTGATAAAGAAATGACTCCTGATACAGATGAAGAGGA
GTTAAAGAGGAGGAGAGTGCTACATTCTCTTTCTACGCCAATTATTACGC
TTCCTCTCCAAGTAGCTCTACCAGAACAAACCGAGCCCGAAGACCTCAGT
ATGTCGACGGGGATGCACTCAAACAATTCGGGAAGCAGCGGCTCCCCGAT
CGTAAGTCCTTGTTTAAAAGAAGAAGAGGACGACATCGAAACAACTGCGA
TATTTCTACAAAGAAGGCCAATGAGTAGATCCTAGTCTAGTTATTTTCGT
GAGATATACGGGTATGTTAAAGTCAAATTATAATTATCCAAAATATATAC
ATAGTCCCTTATCACAGCGTCACTTCCAGAGGAATTGTTTTGGGGAATAA
AAAAGTCTAAAAGGAAAGAAGTAACTGTTACACACTTGCAAATAATTTCT
AATACATATATTATATTTACTTGCACTACAGGTATTGTGTTGAATTTTTT
ATAGAACAATAGATTTCAAATAACAAGTAATAAAATACTCATTTATATTA
CAGTGGAACCCCGATAA

SEQ ID NO:2 shows a contig comprising a further exemplary Diabrotica kruppel DNA:

CTGACAGTCAATAGGTGTAGTTATACGACATTTAGATAGATTTGATAAAC
ATTTACGACGACAGCACGAATATTTTTTATTTTCTTTTGTACATTTTTGT
ATTTTCGTGGTGCGTGATGAAGTGACAATGGACTGGGTTTGTGCGTGGTT
TTGATTTTGTTTTTGCTATTTCGATTTTTGTTTTTGAAATGCCGCTTCTA
CAAGAAAATACTCCAAAAAGAGATTTCCACACATCCCGAGAGATGAAGGC
AGACGAGAAGCCACCGATTAAGCCTCACCAAAGCACCTTCGGGTACCCTG
CGTTTCCAAACCCAGCGATGTTTACCCCATCACAAATCCTAATGGCTAGT
CAACTAATGGCAGCATCCGGCTTGACCATATCTGGAAACCCTGCCTTCTT
CCATCCAGGTTTGATGCCCCTACCATGGGCAGCAAACTCACCTCCTTCCA
ATTCTCCACCCTCACCACCGACCAGCAACGAACCGCTGTCCCCTGCGATG
AGTACAAGAAAACTTAGTCATAATAACAATAATGTTGTTAGTAGTAGTAC
GACGACAGAAATTAGGAAAAGAAAGTATAAGTTAGAAGAAGAAGCCACCA
GTTTACCGCTGCCATCCCCTACGTCTAGTGTATCGCCTCCTTCGGGGTCT
GATATTAAAGATCCTAATAGGGATAAGCAGTTCACCTGTGGAGTATGTAA
TAGATCATTTGGCTACAAACACGTCCTGCAAAATCACGAAAGGACACATA
CCGGAGAGAAACCTTTCGAATGTCCAGAGTGCCACAAAAGGTTCACCAGA
GACCATCACCTTAAGACCCACATGAGACTTCATACAGGAGAACGCCCTTA
TCATTGCGAACACTGTGACAGACAATTCGTCCAAGTTGCCAACCTTCGAA
GACATTTGAGGGTGCATACAGGAGAACGACCTTACTCTTGTGAACACTGT
ACTGCCAAATTCTCAGACTCCAATCAGCTCAAGGCTCACATACTTATCCA
TACCAATGAAAAACCATTTTCGTGTGAAAGGTGTCACAGCAGGTTCAGAA
GGAGGCATCACTTGTTGCACCATAAATGCGGCATGCCTGATAAAGAAATG
ACTCCTGATACAGATGAAGAGGAGTTAAAGAGGAGGAGAGTGCTACATTC
TCTTTCTACGCCAATTATTACGCTTCCTCTCCAAGTAGCTCTACCAGAAC
AAACCGAGCCCGAAGACCTCAGTATGTCGACGGGGATGCACTCAAACAAT
TCGGGAAGCAGCGGCTCCCCGATCGTAAGTCCTTGTTTAAAAGAAGAAGA
GGACGACATCGAAACAACTGCGATATTTCTACAAAGAAGGCCAATGAGTA
GATCCTAGTCTAGTTATTTTCGTGAGATATACGGGTATGTTAAAGTCAAA
TTATAATTATCCAAAATATATACATAGTCCCTTATCACAGCGTCACTTCC
AGAGGAATTGTTTTGGGGAATAAAAAAGTCTAAAAGGAAAGAAGTAACTG
TTACACACTTGCAAATAATTTCTAATACATATATTATATTTACTTGCACT
ACAGGTCTTGTGTTGAATTTTTTATAGAACAATAGATTTCAAATAACAAG
TAATAAAATACTCATTTATATTACAGTGGAACCCCGATAA

SEQ ID NO:3 shows the amino acid sequence of a Diabrotica KRUPPEL polypeptide encoded by an exemplary Diabrotica kruppel DNA:

MKADEKPPIKPHQSTFGYPAFPNPAMFTPSQILMASQLMAASGLTISGNP
AFFHPGLMPLPWAANSPPSNSPPSPPTSNEPLSPAMSTRKLSHNNNNVVS
SSTTTEIRKRKYKLEEEATSLPLPSPTSSVSPPSGSDIKDPNRDKQFTCG
VCNRSFGYKHVLQNHERTHTGEKPFECPECHKRFTRDHHLKTHMRLHTGE
RPYHCEHCDRQFVQVANLRRHLRVHTGERPYSCEHCTAKFSDSNQLKAHI
LIHTNEKPFSCERCHSRFRRRHHLLHHKCGMPDKEMTPDTDEEELKRRRV
LHSLSTPIITLPLQVALPEQTEPEDLSMSTGMHSNNSGSSGSPIVSPCLK
EEEDDIETTAIFLQRRPMSRS

SEQ ID NO:4 shows an exemplary Diabrotica kruppel DNA, referred to herein in some places as kruppel Region 1 (Reg1), which is used in some examples for the production of a dsRNA:

CCTGTGGAGTATGTAATAGATCATTTGGCTACAAACACGTCCTGCAAAAT
CACGAAAGGACACATACCGGAGAGAAACCTTTCGAATGTCCAGAGTGCCA
CAAAAGGTTCACCAGAGACCATCACCTTAAGACCCACATGAGACTTCATA
CAGGAGAACGCCCTTATCATTGCGAACACTGTGACAGACAATTCGTCCAA
GTTGCCAACCTTCGAAGACATTTGAGGGTGCATACAGGAGAACGACCTTA
CTCTTGTGAACACTGTACTGCCAAATTCTCAGACTCCAATC

SEQ ID NO:5 shows the nucleotide sequence of a T7 phage promoter.

SEQ ID NOs:6-9 show primers used to amplify gene regions of a Diabrotica kruppel gene or a GFP gene.

SEQ ID NO:10 shows a partial coding region of a GFP gene.

SEQ ID NO:11 shows an exemplary partial coding region of a YFP gene.

SEQ ID NO:12 shows a DNA sequence of annexin region 1.

SEQ ID NO:13 shows a DNA sequence of annexin region 2.

SEQ ID NO:14 shows a DNA sequence of beta spectrin 2 region 1.

SEQ ID NO:15 shows a DNA sequence of beta spectrin 2 region 2.

SEQ ID NO:16 shows a DNA sequence of mtRP-L4 region 1.

SEQ ID NO:17 shows a DNA sequence of mtRP-L4 region 2.

SEQ ID NOs:18-45 show primers used to amplify gene regions of annexin, beta spectrin 2, mtRP-L4, and YFP for dsRNA synthesis.

SEQ ID NO:46 shows an exemplary DNA comprising an ST-LS1 intron.

SEQ ID NO:47 shows the nucleotide sequence of a T20VN primer oligonucleotide.

SEQ ID NOs:48-52 show primers and probes used for dsRNA transcript expression analyses.

SEQ ID NO:53 shows a nucleotide sequence of a portion of a SpecR coding region used for binary vector backbone detection.

SEQ ID NO:54 shows a nucleotide sequence of an AAD1 coding region used for genomic copy number analysis.

SEQ ID NOs:55-66 show the nucleotide sequences of DNA oligonucleotides used for gene copy number determinations and binary vector backbone detection.

SEQ ID NOs:67-69 show exemplary RNAs transcribed from nucleic acids comprising exemplary kruppel polynucleotides and fragments thereof.

### DETAILED DESCRIPTION

### I. Overview of Several Embodiments

We developed RNA interference (RNAi) as a tool for insect pest management, using one of the most likely target pest species for transgenic plants that express dsRNA; the western corn rootworm. Thus far, most genes proposed as targets for RNAi in rootworm larvae do not achieve their purpose, and those useful targets that have been identified involve those that cause lethality in the larval stage. Herein, we describe RNAi-mediated knockdown of kruppel (kr) in the western corn rootworm, which is shown to disrupt embryonic development when, for example, iRNA molecules are delivered via kruppel dsRNA fed to adult females. Exposure of adult female insects to kruppel dsRNA did not affect adult longevity when administered orally. However, there was almost complete absence of hatching in the eggs collected from females exposed to kruppel dsRNA. In embodiments herein, the ability to deliver kruppel dsRNA by feeding to adult insects confers a pRNAi effect that is very useful for insect (e.g., coleopteran) pest management. Furthermore, the potential to affect multiple target sequences in both larval and adult rootworms may increase opportunities to develop sustainable approaches to insect pest management involving RNAi technologies.

Disclosed herein are methods and compositions for genetic control of coleopteran pest infestations. Methods for identifying one or more gene(s) essential to the life cycle of a coleopteran pest (e.g., gene(s) essential for normal reproductive capacity and/or embryonic and/or larval development) for use as a target gene for RNAi-mediated control of a coleopteran pest population are also provided. DNA plasmid vectors encoding an RNA molecule may be designed to suppress one or more target gene(s) essential for growth, survival, development, and/or reproduction. In some embodiments, the RNA molecule may be capable of forming dsRNA molecules. In some embodiments, methods are provided for post-transcriptional repression of expression or inhibition of a target gene via nucleic acid molecules that are complementary to a coding or non-coding sequence of the target gene in a coleopteran pest. In these and further embodiments, a coleopteran pest may ingest one or more dsRNA, siRNA, shRNA, miRNA, and/or hpRNA molecules transcribed from all or a portion of a nucleic acid molecule that is complementary to a coding or non-coding sequence of a target gene, thereby providing a plant-protective effect.

Some embodiments involve sequence-specific inhibition of expression of target gene products, using dsRNA, siRNA, shRNA, miRNA and/or hpRNA that is complementary to coding and/or non-coding sequences of the target gene(s) to achieve at least partial control of a coleopteran pest. Disclosed is a set of isolated and purified nucleic acid molecules comprising a polynucleotide, for example, as set forth in SEQ ID NOs: 1 and 2, and fragments thereof. In some embodiments, a stabilized dsRNA molecule may be expressed from these polynucleotides, fragments thereof, or a gene comprising one of these polynucleotides, for the post-transcriptional silencing or inhibition of a target gene. In certain embodiments, isolated and purified nucleic acid molecules comprise all or part of any of SEQ ID NOs:1; 2; and 4.

Other embodiments involve a recombinant host cell (e.g., a plant cell) having in its genome at least one recombinant DNA encoding at least one iRNA (e.g., dsRNA) molecule(s). In particular embodiments, the dsRNA molecule(s) may be produced when ingested by a coleopteran pest to post-transcriptionally silence or inhibit the expression of a target gene in the pest or progeny of the pest. The recombinant DNA may comprise, for example, any of SEQ ID NOs:1; 2; and 4, fragments of any of SEQ ID NOs:1; 2; and 4, and a polynucleotide consisting of a partial sequence of a gene comprising one of SEQ ID NOs:1; 2; and 4, and/or complements thereof.

Alternative embodiments involve a recombinant host cell having in its genome a recombinant DNA encoding at least one iRNA (e.g., dsRNA) molecule(s) comprising all or part of SEQ ID NO:67 or SEQ ID NO:68 (e.g., at least one polynucleotide selected from the group consisting of SEQ ID NOs:67-69). When ingested by a coleopteran pest, the iRNA molecule(s) may silence or inhibit the expression of a target kruppel gene (e.g., a DNA comprising all or part of a polynucleotide selected from the group consisting of SEQ ID NO:1 and SEQ ID NO:2) in the pest or progeny of the pest, and thereby result in cessation of reproduction in the pest, and/or growth, development, and/or feeding in progeny of the pest.

In some embodiments, a recombinant host cell having in its genome at least one recombinant DNA encoding at least one RNA molecule capable of forming a dsRNA molecule may be a transformed plant cell. Some embodiments involve transgenic plants comprising such a transformed plant cell. In addition to such transgenic plants, progeny plants of any transgenic plant generation, transgenic seeds, and transgenic plant products, are all provided, each of which comprises recombinant DNA(s). In particular embodiments, an RNA molecule capable of forming a dsRNA molecule may be expressed in a transgenic plant cell. Therefore, in these and other embodiments, a dsRNA molecule may be isolated from a transgenic plant cell. In particular embodiments, the transgenic plant is a plant selected from the group comprising corn (Zea mays), soybean (Glycine max), and plants of the family Poaceae.

Other embodiments involve a method for modulating the expression of a target gene in a coleopteran pest cell. In these and other embodiments, a nucleic acid molecule may be provided, wherein the nucleic acid molecule comprises a polynucleotide encoding an RNA molecule capable of forming a dsRNA molecule. In particular embodiments, a polynucleotide encoding an RNA molecule capable of forming a dsRNA molecule may be operatively linked to a promoter, and may also be operatively linked to a transcription termination sequence. In particular embodiments, a method for modulating the expression of a target gene in a coleopteran pest cell may comprise: (a) transforming a plant cell with a vector comprising a polynucleotide encoding an RNA molecule capable of forming a dsRNA molecule; (b) culturing the transformed plant cell under conditions sufficient to allow for development of a plant cell culture comprising a plurality of transformed plant cells; (c) selecting for a transformed plant cell that has integrated the vector into its genome; and (d) determining that the selected transformed plant cell comprises the RNA molecule capable of forming a dsRNA molecule encoded by the polynucleotide of the vector. A plant may be regenerated from a plant cell that has the vector integrated in its genome and comprises the dsRNA molecule encoded by the polynucleotide of the vector.

Also disclosed is a transgenic plant comprising a vector having a polynucleotide encoding an RNA molecule capable of forming a dsRNA molecule integrated in its genome, wherein the transgenic plant comprises the dsRNA molecule encoded by the polynucleotide of the vector. In particular embodiments, expression of an RNA molecule capable of forming a dsRNA molecule in the plant is sufficient to modulate the expression of a target gene in a cell of a coleopteran pest that contacts the transformed plant or plant cell (for example, by feeding on the transformed plant, a part of the plant (e.g., root) or plant cell) or in a cell of a progeny of the coleopteran pest that contacts the transformed plant or plant cell (for example, by parental transmission), such that reproduction of the pest is inhibited. Transgenic plants disclosed herein may display tolerance and/or protection from coleopteran pest infestations. Particular transgenic plants may display protection and/or enhanced protection from one or more coleopteran pest(s) selected from the group consisting of: WCR; NCR; SCR; MCR; D. balteata LeConte; D. u. tenella; D. speciosa Germar; and D. u. undecimpunctata Mannerheim.

Further disclosed herein are methods for delivery of control agents, such as an iRNA molecule, to a coleopteran pest. Such control agents may cause, directly or indirectly, an impairment in the ability of a coleopteran pest population to feed, grow or otherwise cause damage to a host. In some embodiments, a method is provided comprising delivery of a stabilized dsRNA molecule to a coleopteran pest to suppress at least one target gene in the pest or its progeny, thereby causing parental RNAi and reducing or eliminating plant damage. In some embodiments, a method of inhibiting expression of a target gene in a coleopteran pest may result in cessation of reproduction in the pest, and/or growth, development, and/or feeding in progeny of the pest. In some embodiments, the method may significantly reduce the size of a subsequent pest generation in an infestation, without directly resulting in mortality in the pest(s) that contact the iRNA molecule. In some embodiments, the method may significantly reduce the size of a subsequent pest generation in an infestation, while also resulting in mortality in the pest(s) that contact the iRNA molecule.

In some embodiments, compositions (e.g., a topical composition) are provided that comprise an iRNA (e.g., dsRNA) molecule for use with plants, animals, and/or the environment of a plant or animal to achieve the elimination or reduction of a coleopteran pest infestation. In some embodiments, compositions are provided that include a prokaryote comprising a DNA encoding an iRNA molecule; for example, a transformed bacterial cell. In particular examples, such a transformed bacterial cell may be utilized as a conventional pesticide formulation. In particular embodiments, the composition may be a nutritional composition or resource, or food source, to be fed to the coleopteran pest. Some embodiments comprise making the nutritional composition or food source available to the pest. Ingestion of a composition comprising iRNA molecules may result in the uptake of the molecules by one or more cells of the coleopteran pest, which may in turn result in the inhibition of expression of at least one target gene in cell(s) of the pest or its progeny. Ingestion of or damage to a plant or plant cell by a coleopteran pest infestation may be limited or eliminated in or on any host tissue or environment in which the pest is present by providing one or more compositions comprising an iRNA molecule in the host of the pest.

The compositions and methods disclosed herein may be used together in combinations with other methods and compositions for controlling damage by coleopteran pests. For example, an iRNA molecule as described herein for protecting plants from coleopteran pests may be used in a method comprising the additional use of one or more chemical agents effective against a coleopteran pest, including biopesticides effective against a coleopteran pest, crop rotation, recombinant genetic techniques that exhibit features different from the features of RNAi-mediated methods and RNAi compositions (e.g., recombinant production of proteins in plants that are harmful to a coleopteran pest (e.g., Bt toxins)), and/or recombinant expression of non-parental iRNA molecules (e.g., lethal iRNA molecules that result in mortality, the cessation of growth, development, and/or feeding in the coleopteran pest that ingests the iRNA molecule).

### II. Abbreviations

dsRNA double-stranded ribonucleic acid
GI growth inhibition
GFP green fluorescent protein
NCBI National Center for Biotechnology Information
gDNA genomic deoxyribonucleic acid
iRNA inhibitory ribonucleic acid
ORF open reading frame
RNAi ribonucleic acid interference
miRNA micro ribonucleic acid
siRNA small inhibitory ribonucleic acid
hpRNA hairpin ribonucleic acid
shRNA short hairpin ribonucleic acid
pRNAi parental RNA interference UTR untranslated region
WCR western corn rootworm (Diabrotica virgifera virgifera LeConte)
NCR northern corn rootworm (Diabrotica barberi Smith and Lawrence)
MCR Mexican corn rootworm (Diabrotica virgifera zeae Krysan and Smith)
PCR Polymerase chain reaction
qPCR quantitative polymerase chain reaction
RISC RNA-induced Silencing Complex
RH relative humidity
SCR southern corn rootworm (Diabrotica undecimpunctata howardi Barber)
SEM standard error of the mean
YFP yellow fluorescent protein

### III. Terms

In the description and tables which follow, a number of terms are used. In order to provide a clear and consistent understanding of the specification and claims, including the scope to be given such terms, the following definitions are provided:

Coleopteran pest: As used herein, the term “coleopteran pest” refers to pest insects of the order Coleoptera, including pest insects in the genus Diabrotica, which feed upon agricultural crops and crop products, including corn and other true grasses. In particular examples, a coleopteran pest is selected from a list comprising D. v. virgifera LeConte (WCR); D. barberi Smith and Lawrence (NCR); D. u. howardi (SCR); D. v. zeae (MCR); D. balteata LeConte; D. u. tenella; D. speciosa; and D. u. undecimpunctata Mannerheim.

Contact (with an organism): As used herein, the term “contact with” or “uptake by” an organism (e.g., a coleopteran pest), with regard to a nucleic acid molecule, includes internalization of the nucleic acid molecule into the organism, for example and without limitation: ingestion of the molecule by the organism (e.g., by feeding); contacting the organism with a composition comprising the nucleic acid molecule; and soaking of organisms with a solution comprising the nucleic acid molecule.

Contig: As used herein the term “contig” refers to a DNA sequence that is reconstructed from a set of overlapping DNA segments derived from a single genetic source.

Corn plant: As used herein, the term “corn plant” refers to a plant of the species, Zea mays (maize). The terms “corn plant” and “maize” are used interchangeably herein.

Expression: As used herein, “expression” of a coding polynucleotide (for example, a gene or a transgene) refers to the process by which the coded information of a nucleic acid transcriptional unit (including, e.g., gDNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, northern blot, RT-PCR, western blot, or in vitro, in situ, or in vivo protein activity assay(s).

Genetic material: As used herein, the term “genetic material” includes all genes, and nucleic acid molecules, such as DNA and RNA.

Inhibition: As used herein, the term “inhibition,” when used to describe an effect on a coding polynucleotide (for example, a gene), refers to a measurable decrease in the cellular level of mRNA transcribed from the coding polynucleotide and/or peptide, polypeptide, or protein product of the coding polynucleotide. In some examples, expression of a coding polynucleotide may be inhibited such that expression is approximately eliminated. “Specific inhibition” refers to the inhibition of a target coding polynucleotide without consequently affecting expression of other coding polynucleotides (e.g., genes) in the cell wherein the specific inhibition is being accomplished.

Isolated: An “isolated” biological component (such as a nucleic acid or protein) has been substantially separated, produced apart from, or purified away from other biological components in the cell of the organism in which the component naturally occurs (i.e., other chromosomal and extra-chromosomal DNA and RNA, and proteins), while effecting a chemical or functional change in the component (e.g., a nucleic acid may be isolated from a chromosome by breaking chemical bonds connecting the nucleic acid to the remaining DNA in the chromosome). Nucleic acid molecules and proteins that have been “isolated” include nucleic acid molecules and proteins purified by standard purification methods. The term also embraces nucleic acids and proteins prepared by recombinant expression in a host cell, as well as chemically-synthesized nucleic acid molecules, proteins, and peptides.

Nucleic acid molecule: As used herein, the term “nucleic acid molecule” may refer to a polymeric form of nucleotides, which may include both sense and anti-sense strands of RNA, cDNA, gDNA, and synthetic forms and mixed polymers of the above. A nucleotide or nucleobase may refer to a ribonucleotide, deoxyribonucleotide, or a modified form of either type of nucleotide. A “nucleic acid molecule” as used herein is synonymous with “nucleic acid” and “polynucleotide.” A nucleic acid molecule is usually at least 10 bases in length, unless otherwise specified. By convention, the nucleotide sequence of a nucleic acid molecule is read from the 5′ to the 3′ end of the molecule. The “complement” of a nucleic acid molecule refers to a polynucleotide having nucleobases that may form base pairs with the nucleobases of the nucleic acid molecule (i.e., A-T/U, and G-C).

Some embodiments include nucleic acids comprising a template DNA that is transcribed into an RNA molecule that is the complement of an mRNA molecule. In these embodiments, the complement of the nucleic acid transcribed into the mRNA molecule is present in the 5′ to 3′ orientation, such that RNA polymerase (which transcribes DNA in the 5′ to 3′ direction) will transcribe a nucleic acid from the complement that can hybridize to the mRNA molecule. Unless explicitly stated otherwise, or it is clear to be otherwise from the context, the term “complement” therefore refers to a polynucleotide having nucleobases, from 5′ to 3′, that may form base pairs with the nucleobases of a reference nucleic acid. Similarly, unless it is explicitly stated to be otherwise (or it is clear to be otherwise from the context), the “reverse complement” of a nucleic acid refers to the complement in reverse orientation. The foregoing is demonstrated in the following illustration:

| | |
|---|---|
| ATGATGATG | polynucleotide |
| TACTACTAC | “complement” of the polynucleotide |
| CATCATCAT | “reverse complement” of the |
| | polynucleotide |

Some embodiments of the invention may include hairpin RNA-forming RNAi molecules. In these RNAi molecules, both the complement of a nucleic acid to be targeted by RNA interference and the reverse complement may be found in the same molecule, such that the single-stranded RNA molecule may “fold over” and hybridize to itself over region comprising the complementary and reverse complementary polynucleotides.

“Nucleic acid molecules” include all polynucleotides, for example: single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), shRNA (small hairpin RNA), mRNA (messenger RNA), miRNA (micro-RNA), hpRNA (hairpin RNA), tRNA (transfer RNAs, whether charged or discharged with a corresponding acylated amino acid), and cRNA (complementary RNA). The term “deoxyribonucleic acid” (DNA) is inclusive of cDNA, gDNA, and DNA-RNA hybrids. The terms “polynucleotide” and “nucleic acid,” and “fragments” thereof will be understood by those in the art as a term that includes both gDNAs, ribosomal RNAs, transfer RNAs, messenger RNAs, operons, and smaller engineered polynucleotides that encode or may be adapted to encode, peptides, polypeptides, or proteins.

Oligonucleotide: An oligonucleotide is a short nucleic acid polymer. Oligonucleotides may be formed by cleavage of longer nucleic acid segments, or by polymerizing individual nucleotide precursors. Automated synthesizers allow the synthesis of oligonucleotides up to several hundred bases in length. Because oligonucleotides may bind to a complementary nucleic acid, they may be used as probes for detecting DNA or RNA. Oligonucleotides composed of DNA (oligodeoxyribonucleotides) may be used in PCR, a technique for the amplification of DNAs. In PCR, the oligonucleotide is typically referred to as a “primer,” which allows a DNA polymerase to extend the oligonucleotide and replicate the complementary strand.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

As used herein with respect to DNA, the term “coding polynucleotide,” “structural polynucleotide,” or “structural nucleic acid molecule” refers to a polynucleotide that is ultimately translated into a polypeptide, via transcription and mRNA, when placed under the control of appropriate regulatory elements. With respect to RNA, the term “coding polynucleotide” refers to a polynucleotide that is translated into a peptide, polypeptide, or protein. The boundaries of a coding polynucleotide are determined by a translation start codon at the 5′-terminus and a translation stop codon at the 3′-terminus. Coding polynucleotides include, but are not limited to: gDNA; cDNA; EST; and recombinant polynucleotides.

As used herein, “transcribed non-coding polynucleotide” refers to segments of mRNA molecules such as 5′UTR, 3′UTR and intron segments that are not translated into a peptide, polypeptide, or protein. Further, “transcribed non-coding polynucleotide” refers to a nucleic acid that is transcribed into an RNA that functions in the cell, for example, structural RNAs (e.g., ribosomal RNA (rRNA) as exemplified by 5S rRNA, 5.8S rRNA, 16S rRNA, 18S rRNA, 23S rRNA, and 28S rRNA, and the like); transfer RNA (tRNA); and small nuclear RNAs (snRNAs), such as U4, U5, U6, and the like. Transcribed non-coding polynucleotides also include, for example and without limitation, small RNAs (sRNA), which term is often used to describe small bacterial non-coding RNAs; small nucleolar RNAs (snoRNA); microRNAs; small interfering RNAs (siRNA); Piwi-interacting RNAs (piRNA); and long non-coding RNAs. Further still, “transcribed non-coding polynucleotide” refers to a polynucleotide that may natively exist as an intragenic “linker” in a nucleic acid and which is transcribed into an RNA molecule.

Lethal RNA interference: As used herein, the term “lethal RNA interference” refers to RNA interference that results in death or a reduction in viability of the subject individual to which, for example, a dsRNA, miRNA, siRNA, shRNA, and/or hpRNA is delivered.

Parental RNA interference: As used herein, the term “parental RNA interference” (pRNAi) refers to a RNA interference phenotype that is observable in progeny of the subject (e.g., a coleopteran pest) to which, for example, a dsRNA, miRNA, siRNA, shRNA, and/or hpRNA is delivered. In some embodiments, pRNAi comprises the delivery of a dsRNA to a coleopteran pest, wherein the pest is thereby rendered less able to produce viable offspring. A nucleic acid that initiates pRNAi may or may not increase the incidence of mortality in a population into which the nucleic acid is delivered. In certain examples, the nucleic acid that initiates pRNAi does not increase the incidence of mortality in the population into which the nucleic acid is delivered. For example, a population of coleopteran pests may be fed one or more nucleic acids that initiate pRNAi, wherein the pests survive and mate but produce eggs that are less able to hatch viable progeny than eggs produced by pests of the same species that are not fed the nucleic acid(s). In one mechanism of pRNAi, parental RNAi delivered to a female is able to knock down zygotic gene expression in offspring embryos of the female. Bucher et al. (2002) Curr. Biol. 12(3):R85-6.

Genome: As used herein, the term “genome” refers to chromosomal DNA found within the nucleus of a cell, and also refers to organelle DNA found within subcellular components of the cell. In some embodiments of the invention, a DNA molecule may be introduced into a plant cell, such that the DNA molecule is integrated into the genome of the plant cell. In these and further embodiments, the DNA molecule may be either integrated into the nuclear DNA of the plant cell, or integrated into the DNA of the chloroplast or mitochondrion of the plant cell. The term “genome,” as it applies to bacteria, refers to both the chromosome and plasmids within the bacterial cell. In some embodiments of the invention, a DNA molecule may be introduced into a bacterium such that the DNA molecule is integrated into the genome of the bacterium. In these and further embodiments, the DNA molecule may be either chromosomally-integrated or located as or in a stable plasmid.

Sequence identity: The term “sequence identity” or “identity,” as used herein in the context of two polynucleotides or polypeptides, refers to the residues in the sequences of the two molecules that are the same when aligned for maximum correspondence over a specified comparison window.

As used herein, the term “percentage of sequence identity” may refer to the value determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default BLOSUM62 matrix set to default parameters. Nucleic acids with even greater sequence similarity to the sequences of the reference polynucleotides will show increasing percentage identity when assessed by this method.

Specifically hybridizable/Specifically complementary: As used herein, the terms “Specifically hybridizable” and “Specifically complementary” are terms that indicate a sufficient degree of complementarity such that stable and specific binding occurs between the nucleic acid molecule and a target nucleic acid molecule. Hybridization between two nucleic acid molecules involves the formation of an anti-parallel alignment between the nucleobases of the two nucleic acid molecules. The two molecules are then able to form hydrogen bonds with corresponding bases on the opposite strand to form a duplex molecule that, if it is sufficiently stable, is detectable using methods well known in the art. A polynucleotide need not be 100% complementary to its target nucleic acid to be specifically hybridizable. However, the amount of complementarity that must exist for hybridization to be specific is a function of the hybridization conditions used.

Hybridization conditions resulting in particular degrees of stringency will vary depending upon the nature of the hybridization method of choice and the composition and length of the hybridizing nucleic acids. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are known to those of ordinary skill in the art, and are discussed, for example, in Sambrook et al. (ed.) Molecular Cloning: A Laboratory Manual, 2^{nd} ed., vol. 1-3, Cold Spring Harbor Laboratory Press, Cold Spring Harbor, N. Y., 1989, chapters 9 and 11; and Hames and Higgins (eds.) Nucleic Acid Hybridization, IRL Press, Oxford, 1985. Further detailed instruction and guidance with regard to the hybridization of nucleic acids may be found, for example, in Tijssen, “Overview of principles of hybridization and the strategy of nucleic acid probe assays,” in Laboratory Techniques in Biochemistry and Molecular Biology-Hybridization with Nucleic Acid Probes, Part I, Chapter 2, Elsevier, N.Y., 1993; and Ausubel et al., Eds., Current Protocols in Molecular Biology, Chapter 2, Greene Publishing and Wiley-Interscience, N.Y., 1995.

As used herein, “stringent conditions” encompass conditions under which hybridization will only occur if there is less than 20% mismatch between the sequence of the hybridization molecule and a homologous polynucleotide within the target nucleic acid molecule. “Stringent conditions” include further particular levels of stringency. Thus, as used herein, “moderate stringency” conditions are those under which molecules with more than 20% sequence mismatch will not hybridize; conditions of “high stringency” are those under which sequences with more than 10% mismatch will not hybridize; and conditions of “very high stringency” are those under which sequences with more than 5% mismatch will not hybridize.

The following are representative, non-limiting hybridization conditions.

High Stringency condition (detects polynucleotides that share at least 90% sequence identity): Hybridization in 5×SSC buffer at 65° C. for 16 hours; wash twice in 2×SSC buffer at room temperature for 15 minutes each; and wash twice in 0.5×SSC buffer at 65° C. for 20 minutes each. Moderate Stringency condition (detects polynucleotides that share at least 80% sequence identity): Hybridization in 5×-6×SSC buffer at 65-70° C. for 16-20 hours; wash twice in 2×SSC buffer at room temperature for 5-20 minutes each; and wash twice in 1×SSC buffer at 55-70° C. for 30 minutes each. Non-stringent control condition (polynucleotides that share at least 50% sequence identity will hybridize): Hybridization in 6×SSC buffer at room temperature to 55° C. for 16-20 hours; wash at least twice in 2×-3×SSC buffer at room temperature to 55° C. for 20-30 minutes each.

As used herein, the term “substantially homologous” or “substantial homology,” with regard to a nucleic acid, refers to a polynucleotide having contiguous nucleobases that hybridize under stringent conditions to the reference nucleic acid. For example, nucleic acids that are substantially homologous to a reference nucleic acid of any of SEQ ID NOs:1, 2, and 4 are those nucleic acids that hybridize under stringent conditions (e.g., the Moderate Stringency conditions set forth, supra) to the reference nucleic acid of any of SEQ ID NOs:1, 2, and 4. Substantially homologous polynucleotides may have at least 80% sequence identity. For example, substantially homologous polynucleotides may have from about 80% to 100% sequence identity, such as 79%; 80%; about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target polynucleotides under conditions where specific binding is desired, for example, under stringent hybridization conditions.

As used herein, the term “ortholog” refers to a gene in two or more species that has evolved from a common ancestral nucleic acid, and may retain the same function in the two or more species.

As used herein, two nucleic acid molecules are said to exhibit “complete complementarity” when every nucleotide of a polynucleotide read in the 5′ to 3′ direction is complementary to every nucleotide of the other polynucleotide when read in the 3′ to 5′ direction. A polynucleotide that is complementary to a reference polynucleotide will exhibit a sequence identical to the reverse complement of the reference polynucleotide. These terms and descriptions are well defined in the art and are easily understood by those of ordinary skill in the art.

Operably linked: A first polynucleotide is operably linked with a second polynucleotide when the first polynucleotide is in a functional relationship with the second polynucleotide. When recombinantly produced, operably linked polynucleotides are generally contiguous, and, where necessary to join two protein-coding regions, in the same reading frame (e.g., in a translationally fused ORF). However, nucleic acids need not be contiguous to be operably linked.

The term, “operably linked,” when used in reference to a regulatory genetic element and a coding polynucleotide, means that the regulatory element affects the expression of the linked coding polynucleotide. “Regulatory elements,” or “control elements,” refer to polynucleotides that influence the timing and level/amount of transcription, RNA processing or stability, or translation of the associated coding polynucleotide. Regulatory elements may include promoters; translation leaders; introns; enhancers; stem-loop structures; repressor binding polynucleotides; polynucleotides with a termination sequence; polynucleotides with a polyadenylation recognition sequence; etc. Particular regulatory elements may be located upstream and/or downstream of a coding polynucleotide operably linked thereto. Also, particular regulatory elements operably linked to a coding polynucleotide may be located on the associated complementary strand of a double-stranded nucleic acid molecule.

Promoter: As used herein, the term “promoter” refers to a region of DNA that may be upstream from the start of transcription, and that may be involved in recognition and binding of RNA polymerase and other proteins to initiate transcription. A promoter may be operably linked to a coding polynucleotide for expression in a cell, or a promoter may be operably linked to a polynucleotide encoding a signal peptide which may be operably linked to a coding polynucleotide for expression in a cell. A “plant promoter” may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters which initiate transcription only in certain tissues are referred to as “tissue-specific”. A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter which may be active under most environmental conditions or in most tissue or cell types.

Any inducible promoter can be used in some embodiments of the invention. See Ward et al. (1993) Plant Mol. Biol. 22:361-366. With an inducible promoter, the rate of transcription increases in response to an inducing agent. Exemplary inducible promoters include, but are not limited to: Promoters from the ACEI system that respond to copper; In2 gene from maize that responds to benzenesulfonamide herbicide safeners; Tet repressor from Tn10; and the inducible promoter from a steroid hormone gene, the transcriptional activity of which may be induced by a glucocorticosteroid hormone (Schena et al. (1991) Proc. Natl. Acad. Sci. USA 88:0421).

Exemplary constitutive promoters include, but are not limited to: Promoters from plant viruses, such as the 35S promoter from Cauliflower Mosaic Virus (CaMV); promoters from rice actin genes; ubiquitin promoters; pEMU; MAS; maize H3 histone promoter; and the ALS promoter, XbaI/NcoI fragment 5′ to the Brassica napus ALS3 structural gene (or a polynucleotide similar to said XbaI/NcoI fragment) (International PCT Publication No. WO96/30530).

Additionally, any tissue-specific or tissue-preferred promoter may be utilized in some embodiments of the invention. Plants transformed with a nucleic acid molecule comprising a coding polynucleotide operably linked to a tissue-specific promoter may produce the product of the coding polynucleotide exclusively, or preferentially, in a specific tissue. Exemplary tissue-specific or tissue-preferred promoters include, but are not limited to: A seed-preferred promoter, such as that from the phaseolin gene; a leaf-specific and light-induced promoter such as that from cab or rubisco; an anther-specific promoter such as that from LAT52; a pollen-specific promoter such as that from Zm13; and a microspore-preferred promoter such as that from apg.

Soybean plant: As used herein, the term “soybean plant” refers to a plant of a Glycine species; for example, G. max.

Transformation: As used herein, the term “transformation” or “transduction” refers to the transfer of one or more nucleic acid molecule(s) into a cell. A cell is “transformed” by a nucleic acid molecule transduced into the cell when the nucleic acid molecule becomes stably replicated by the cell, either by incorporation of the nucleic acid molecule into the cellular genome, or by episomal replication. As used herein, the term “transformation” encompasses all techniques by which a nucleic acid molecule can be introduced into such a cell. Examples include, but are not limited to: transfection with viral vectors; transformation with plasmid vectors; electroporation (Fromm et al. (1986) Nature 319:791-3); lipofection (Feigner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; and microprojectile bombardment (Klein et al. (1987) Nature 327:70).

Transgene: An exogenous nucleic acid. In some examples, a transgene may be a DNA that encodes one or both strand(s) of an RNA capable of forming a dsRNA molecule that comprises a polynucleotide that is complementary to a nucleic acid molecule found in a coleopteran pest. In further examples, a transgene may be an antisense polynucleotide, wherein expression of the antisense polynucleotide inhibits expression of a target nucleic acid, thereby producing a parental RNAi phenotype. In still further examples, a transgene may be a gene (e.g., a herbicide-tolerance gene, a gene encoding an industrially or pharmaceutically useful compound, or a gene encoding a desirable agricultural trait). In these and other examples, a transgene may contain regulatory elements operably linked to a coding polynucleotide of the transgene (e.g., a promoter).

Vector: A nucleic acid molecule as introduced into a cell, for example, to produce a transformed cell. A vector may include genetic elements that permit it to replicate in the host cell, such as an origin of replication. Examples of vectors include, but are not limited to: a plasmid; cosmid; bacteriophage; or virus that carries exogenous DNA into a cell. A vector may also include one or more genes, including ones that produce antisense molecules, and/or selectable marker genes and other genetic elements known in the art. A vector may transduce, transform, or infect a cell, thereby causing the cell to express the nucleic acid molecules and/or proteins encoded by the vector. A vector optionally includes materials to aid in achieving entry of the nucleic acid molecule into the cell (e.g., a liposome, protein coating, etc.).

Yield: A stabilized yield of about 100% or greater relative to the yield of check varieties in the same growing location growing at the same time and under the same conditions. In particular embodiments, “improved yield” or “improving yield” means a cultivar having a stabilized yield of 105% or greater relative to the yield of check varieties in the same growing location containing significant densities of the coleopteran pests that are injurious to that crop growing at the same time and under the same conditions, which are targeted by the compositions and methods herein.

Unless specifically indicated or implied, the terms “a,” “an,” and “the” signify “at least one,” as used herein.

Unless otherwise specifically explained, all technical and scientific terms used herein have the same meaning as commonly understood by those of ordinary skill in the art to which this disclosure belongs. Definitions of common terms in molecular biology can be found in, for example, Lewin's Genes X, Jones & Bartlett Publishers, 2009 (ISBN 10 0763766321); Krebs et al. (eds.), The Encyclopedia of Molecular Biology, Blackwell Science Ltd., 1994 (ISBN 0-632-02182-9); and Meyers R. A. (ed.), Molecular Biology and Biotechnology: A Comprehensive Desk Reference, VCH Publishers, Inc., 1995 (ISBN 1-56081-569-8). All percentages are by weight and all solvent mixture proportions are by volume unless otherwise noted. All temperatures are in degrees Celsius.

### IV. Nucleic Acid Molecules Comprising a Coleopteran Pest Polynucleotide

### A. Overview

Described herein are nucleic acid molecules useful for the control of coleopteran pests. Described nucleic acid molecules include target polynucleotides (e.g., native genes, and non-coding polynucleotides), dsRNAs, siRNAs, shRNAs, hpRNAs, and miRNAs. For example, dsRNA, siRNA, miRNA, shRNA, and/or hpRNA molecules are described in some embodiments that may be specifically complementary to all or part of one or more native nucleic acids in a coleopteran pest. In these and further embodiments, the native nucleic acid(s) may be one or more target gene(s), the product of which may be, for example and without limitation: involved in a reproductive process or involved in larval development. Nucleic acid molecules described herein, when introduced into a cell (e.g., through parental transmission) comprising at least one native nucleic acid(s) to which the nucleic acid molecules are specifically complementary, may initiate RNAi in the cell, and consequently reduce or eliminate expression of the native nucleic acid(s). In some examples, reduction or elimination of the expression of a target gene by a nucleic acid molecule specifically complementary thereto may result in reduction or cessation of reproduction in the coleopteran pest, and/or growth, development, and/or feeding in progeny of the pest. These methods may significantly reduce the size of a subsequent pest generation in an infestation, for example, without directly resulting in mortality in the pest(s) that contact the iRNA molecule.

In some embodiments, at least one target gene in a coleopteran pest may be selected, wherein the target gene comprises a kruppel polynucleotide. In particular examples, a target gene in a coleopteran pest is selected, wherein the target gene comprises a polynucleotide selected from among SEQ ID NOs:1, 2, and 4.

The western corn rootworm kruppel represents a sequence of 1617 bp (SEQ ID NO:1), 1640 bp (SEQ ID NO:2), and 371 amino acids (KRUPPEL protein (SEQ ID NO:3)). Within this sequence, four H2C2 type zinc finger domains were predicted at amino acid positions 162-186, 189-213, 217-241, and 246-269 in agreement with its role as a DNA binding transcription factor. See, e.g., Zuo (1991) Genes Dev 5:254-64. When searched in NCBI database using the BLASTp algorithm, the most similar sequence was from Tribolium castaneum, and it exhibited only 65 percent sequence identity (NP_001034527.2).

In some embodiments, a target gene may be a nucleic acid molecule comprising a polynucleotide that can be reverse translated in silico to a polypeptide comprising a contiguous amino acid sequence that is at least about 85% identical (e.g., at least 84%, 85%, about 90%, about 95%, about 96%, about 97%, about 98%, about 99%, about 100%, or 100% identical) to the amino acid sequence of a protein product of a kruppel polynucleotide. A target gene may be any nucleic acid in a coleopteran pest, the post-transcriptional inhibition of which has a deleterious effect on the capacity of the pest to produce viable offspring, for example, to provide a protective benefit against the pest to a plant. In particular examples, a target gene is a nucleic acid molecule comprising a polynucleotide that can be reverse translated in silico to a polypeptide comprising a contiguous amino acid sequence that is at least about 85% identical, about 90% identical, about 95% identical, about 96% identical, about 97% identical, about 98% identical, about 99% identical, about 100% identical, or 100% identical to the amino acid sequence that is the in silico translation product of SEQ ID NO:1 and/or SEQ ID NO:2 (e.g., SEQ ID NO:3).

Provided in some embodiments are DNAs, the expression of which results in an RNA molecule comprising a polynucleotide that is specifically complementary to all or part of a native RNA molecule that is encoded by a coding polynucleotide in a coleopteran pest. In some embodiments, after ingestion of the expressed RNA molecule by a coleopteran pest, down-regulation of the coding polynucleotide in cells of the pest, or in cells of progeny of the pest, may be obtained. In particular embodiments, down-regulation of the coding polynucleotide in cells of the coleopteran pest may result in reduction or cessation of reproduction and/or proliferation in the pest, and/or growth, development, and/or feeding in progeny of the pest.

In some embodiments, target polynucleotides include transcribed non-coding RNAs, such as 5′UTRs; 3′UTRs; spliced leaders; introns; outrons (e.g., 5′UTR RNA subsequently modified in trans splicing); donatrons (e.g., non-coding RNA required to provide donor sequences for trans splicing); and other non-coding transcribed RNA of target coleopteran pest genes. Such polynucleotides may be derived from both mono-cistronic and poly-cistronic genes.

Thus, also described herein in connection with some embodiments are iRNA molecules (e.g., dsRNAs, siRNAs, miRNAs, shRNAs, and hpRNAs) that comprise at least one polynucleotide that is specifically complementary to all or part of a target nucleic acid in a coleopteran pest. In some embodiments an iRNA molecule may comprise polynucleotide(s) that are complementary to all or part of a plurality of target nucleic acids; for example, 2, 3, 4, 5, 6, 7, 8, 9, 10, or more target nucleic acids. In particular embodiments, an iRNA molecule may be produced in vitro, or in vivo by a genetically-modified organism, such as a plant or bacterium. Also disclosed are cDNAs that may be used for the production of dsRNA molecules, siRNA molecules, miRNA molecules, shRNA molecules, and/or hpRNA molecules that are specifically complementary to all or part of a target nucleic acid in a coleopteran pest. Further described are recombinant DNA constructs for use in achieving stable transformation of particular host targets. Transformed host targets may express effective levels of dsRNA, siRNA, miRNA, shRNA, and/or hpRNA molecules from the recombinant DNA constructs. Therefore, also described is a plant transformation vector comprising at least one polynucleotide operably linked to a heterologous promoter functional in a plant cell, wherein expression of the polynucleotide(s) results in an RNA molecule comprising a string of contiguous nucleobases that is specifically complementary to all or part of a target nucleic acid in a coleopteran pest.

In particular examples, nucleic acid molecules useful for the control of coleopteran pests may include: all or part of a native nucleic acid isolated from Diabrotica comprising a kruppel polynucleotide (e.g., any of SEQ ID NOs:1 and 2); DNAs that when expressed result in an RNA molecule comprising a polynucleotide that is specifically complementary to all or part of a native RNA molecule that is encoded by kruppel; iRNA molecules (e.g., dsRNAs, siRNAs, miRNAs, shRNAs, and hpRNAs) that comprise at least one polynucleotide that is specifically complementary to all or part of an RNA molecule encoded by kruppel; cDNAs that may be used for the production of dsRNA molecules, siRNA molecules, miRNA molecules, shRNA molecules, and/or hpRNA molecules that are specifically complementary to all or part of an RNA molecule encoded by kruppel; and recombinant DNA constructs for use in achieving stable transformation of particular host targets, wherein a transformed host target comprises one or more of the foregoing nucleic acid molecules.

### B. Nucleic Acid Molecules

The present invention provides, inter alia, iRNA (e.g., dsRNA, siRNA, miRNA, shRNA, and hpRNA) molecules that inhibit target gene expression in a cell, tissue, or organ of a coleopteran pest; and DNA molecules capable of being expressed as an iRNA molecule in a cell or microorganism to inhibit target gene expression in a cell, tissue, or organ of a coleopteran pest.

Some embodiments of the invention provide an isolated nucleic acid molecule comprising at least one (e.g., one, two, three, or more) polynucleotide(s) selected from the group consisting of: SEQ ID NO:1; the complement of SEQ ID NO:1; SEQ ID NO:2; the complement of SEQ ID NO:2; a fragment of at least 15 contiguous nucleotides (e.g., at least 19 contiguous nucleotides) of SEQ ID NO:1 and/or SEQ ID NO:2 (e.g., SEQ ID NO:4); the complement of a fragment of at least 15 contiguous nucleotides of SEQ ID NO:1 and/or SEQ ID NO:2; a native coding polynucleotide of a Diabrotica organism (e.g., WCR) comprising SEQ ID NO:1 and/or SEQ ID NO:2; the complement of a native coding polynucleotide of a Diabrotica organism comprising SEQ ID NO:1 and/or SEQ ID NO:2; a fragment of at least 15 contiguous nucleotides of a native coding polynucleotide of a Diabrotica organism comprising SEQ ID NO:1 and/or SEQ ID NO:2; and the complement of a fragment of at least 15 contiguous nucleotides of a native coding polynucleotide of a Diabrotica organism comprising SEQ ID NO:1 and/or SEQ ID NO:2. In particular embodiments, contact with or uptake by a coleopteran pest of the isolated polynucleotide inhibits the growth, development, reproduction and/or feeding of the pest.

In some embodiments, an isolated nucleic acid molecule of the invention may comprise at least one (e.g., one, two, three, or more) polynucleotide(s) selected from the group consisting of: SEQ ID NO:67; the complement of SEQ ID NO:67; SEQ ID NO:68; the complement of SEQ ID NO:68; SEQ ID NO:69; the complement of SEQ ID NO:69; a fragment of at least 15 contiguous nucleotides of any of SEQ ID NOs:67-69; the complement of a fragment of at least 15 contiguous nucleotides of any of SEQ ID NOs:67-69; a native polyribonucleotide transcribed in a Diabrotica organism from a gene comprising SEQ ID NO:1 and/or SEQ ID NO:2; the complement of a native polyribonucleotide transcribed in a Diabrotica organism from a gene comprising SEQ ID NO:1 and/or SEQ ID NO:2; a fragment of at least 15 contiguous nucleotides of a native polyribonucleotide transcribed in a Diabrotica organism from a gene comprising SEQ ID NO:1 and/or SEQ ID NO:2; and the complement of a fragment of at least 15 contiguous nucleotides of a native polyribonucleotide transcribed in a Diabrotica organism from a gene comprising SEQ ID NO:1 and/or SEQ ID NO:2. In particular embodiments, contact with or uptake by a coleopteran pest of the isolated polynucleotide inhibits the growth, development, reproduction and/or feeding of the pest.

In other embodiments, a nucleic acid molecule of the invention may comprise at least one (e.g., one, two, three, or more) DNA(s) capable of being expressed as an iRNA molecule in a cell or microorganism to inhibit target gene expression in a cell, tissue, or organ of a coleopteran pest. Such DNA(s) may be operably linked to a promoter that functions in a cell comprising the DNA molecule to initiate or enhance the transcription of the encoded RNA capable of forming a dsRNA molecule(s). In one embodiment, the at least one (e.g., one, two, three, or more) DNA(s) may be derived from the polynucleotide of SEQ ID NO:1 and/or SEQ ID NO:2. Derivatives of SEQ ID NO:1 and/or SEQ ID NO:2 includes fragments of SEQ ID NO:1 and/or SEQ ID NO:2. In some embodiments, such a fragment may comprise, for example, at least about 15 contiguous nucleotides of SEQ ID NO:1 and/or SEQ ID NO:2, or a complement thereof. Thus, such a fragment may comprise, for example, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200 or more contiguous nucleotides of SEQ ID NO:1 and/or SEQ ID NO:2, or a complement thereof. In some examples, such a fragment may comprise, for example, at least 19 contiguous nucleotides (e.g., 19, 20, 21, 22, 23, 24, 25, 26, 27, 28, 29, or 30 contiguous nucleotides) of SEQ ID NO:1 and/or SEQ ID NO:2, or a complement thereof.

Particular embodiments comprise introducing partially- or fully-stabilized dsRNA molecules into a coleopteran pest to inhibit expression of a target gene in a cell, tissue, or organ of the coleopteran pest. When expressed as an iRNA molecule (e.g., dsRNA, siRNA, miRNA, shRNA, and hpRNA) and taken up by a coleopteran pest, polynucleotides comprising one or more fragments of either of SEQ ID NOs:1 and 2, and the complements thereof, may cause one or more of death, developmental arrest, growth inhibition, change in sex ratio, reduction in brood size, cessation of infection, and/or cessation of feeding by a coleopteran pest. In particular examples, polynucleotides comprising one or more fragments (e.g., polynucleotides including about 15 to about 300 nucleotides) of either of SEQ ID NOs:1 and 2, and the complements thereof, cause a reduction in the capacity of an existing generation of the pest to produce a subsequent generation of the pest.

In certain embodiments, dsRNA molecules provided by the invention comprise polynucleotides complementary to a transcript from a target gene comprising SEQ ID NO:1 and/or SEQ ID NO:2, and/or polynucleotides complementary to a fragment of SEQ ID NO:1 and/or SEQ ID NO:2, the inhibition of which target gene in a coleopteran pest results in the reduction or removal of a polypeptide or polynucleotide agent that is essential for the pest's or the pest's progeny's growth, development, or other biological function. A selected polynucleotide may exhibit from about 80% to about 100% sequence identity to SEQ ID NOs:1, 2, and/or 4, a contiguous fragment of SEQ ID NO:1 and/or SEQ ID NO:2, or the complement of either of the foregoing. For example, a selected polynucleotide may exhibit 79%; 80%; about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; or about 100% sequence identity to SEQ ID NOs:1, 2, and/or 4, a contiguous fragment of SEQ ID NO:1 and/or SEQ ID NO:2, or the complement of either of the foregoing.

In some embodiments, a DNA molecule capable of being expressed as an iRNA molecule in a cell or microorganism to inhibit target gene expression may comprise a single polynucleotide that is specifically complementary to all or part of a native polynucleotide found in one or more target coleopteran pest species, or the DNA molecule can be constructed as a chimera from a plurality of such specifically complementary polynucleotides.

In alternative embodiments, a nucleic acid molecule may comprise a first and a second polynucleotide separated by a “linker.” A linker may be a region comprising any sequence of nucleotides that facilitates secondary structure formation between the first and second polynucleotides, where this is desired. In one embodiment, the linker is part of a sense or antisense coding polynucleotide for mRNA. The linker may alternatively comprise any combination of nucleotides or homologues thereof that are capable of being linked covalently to a nucleic acid molecule. In some examples, the linker may comprise an intron (e.g., as ST-LS1 intron).

For example, in some embodiments, the DNA molecule may comprise a polynucleotide coding for one or more different RNA molecules, wherein each of the different RNA molecules comprises a first polynucleotide and a second polynucleotide, wherein the first and second polynucleotides are complementary to each other. The first and second polynucleotides may be connected within an RNA molecule by a linker. The linker may constitute part of the first polynucleotide or the second polynucleotide. Expression of an RNA molecule comprising the first and second nucleotide polynucleotides may lead to the formation of a dsRNA molecule of the present invention, by specific intramolecular base-pairing of the first and second nucleotide polynucleotides. The first polynucleotide or the second polynucleotide may be substantially identical to a polynucleotide native to a coleopteran pest (e.g., a target gene, or transcribed non-coding polynucleotide), a derivative thereof, or a complementary polynucleotide thereto.

dsRNA nucleic acid molecules comprise double strands of polymerized ribonucleotides, and may include modifications to either the phosphate-sugar backbone or the nucleoside. Modifications in RNA structure may be tailored to allow specific inhibition. In one embodiment, dsRNA molecules may be modified through a ubiquitous enzymatic process so that siRNA molecules may be generated. This enzymatic process may utilize an RNase III enzyme, such as DICER in eukaryotes, either in vitro or in vivo. See Elbashir et al. (2001) Nature 411:494-8; and Hamilton and Baulcombe (1999) Science 286(5441):950-2. DICER or functionally-equivalent RNase III enzymes cleave larger dsRNA strands and/or hpRNA molecules into smaller oligonucleotides (e.g., siRNAs), each of which is about 19-25 nucleotides in length. The siRNA molecules produced by these enzymes have 2 to 3 nucleotide 3′ overhangs, and 5′ phosphate and 3′ hydroxyl termini. The siRNA molecules generated by RNase III enzymes are unwound and separated into single-stranded RNA in the cell. The siRNA molecules then specifically hybridize with RNAs transcribed from a target gene, and both RNA molecules are subsequently degraded by an inherent cellular RNA-degrading mechanism. This process may result in the effective degradation or removal of the RNA encoded by the target gene in the target organism. The outcome is the post-transcriptional silencing of the targeted gene. In some embodiments, siRNA molecules produced by endogenous RNase III enzymes from heterologous nucleic acid molecules may efficiently mediate the down-regulation of target genes in coleopteran pests.

In some embodiments, a nucleic acid molecule of the invention may include at least one non-naturally occurring polynucleotide that can be transcribed into a single-stranded RNA molecule capable of forming a dsRNA molecule in vivo through intermolecular hybridization. Such dsRNAs typically self-assemble, and can be provided in the nutrition source of a coleopteran pest to achieve the post-transcriptional inhibition of a target gene. In these and further embodiments, a nucleic acid molecule of the invention may comprise two different non-naturally occurring polynucleotides, each of which is specifically complementary to a different target gene in a coleopteran pest. When such a nucleic acid molecule is provided as a dsRNA molecule to a coleopteran pest, the dsRNA molecule inhibits the expression of at least two different target genes in the pest.

### C. Obtaining Nucleic Acid Molecules

A variety of polynucleotides in coleopteran pests may be used as targets for the design of nucleic acid molecules of the invention, such as iRNAs and DNA molecules encoding iRNAs. Selection of native polynucleotides is not, however, a straight-forward process. Only a small number of native polynucleotides in the coleopteran pest will be effective targets. For example, it cannot be predicted with certainty whether a particular native polynucleotide can be effectively down-regulated by nucleic acid molecules of the invention, or whether down-regulation of a particular native polynucleotide will have a detrimental effect on the growth, viability, proliferation, and/or reproduction of the coleopteran pest. The vast majority of native coleopteran pest polynucleotides, such as ESTs isolated therefrom (e.g., the coleopteran pest polynucleotides listed in U.S. Pat. No. 7,612,194), do not have a detrimental effect on the growth, viability, proliferation, and/or reproduction of the pest. Neither is it predictable which of the native polynucleotides that may have a detrimental effect on a coleopteran pest are able to be used in recombinant techniques for expressing nucleic acid molecules complementary to such native polynucleotides in a host plant and providing the detrimental effect on the pest upon feeding without causing harm to the host plant.

In some embodiments, nucleic acid molecules of the invention (e.g., dsRNA molecules to be provided in the host plant of a coleopteran pest) are selected to target cDNAs that encode proteins or parts of proteins essential for coleopteran pest reproduction and/or development, such as polypeptides involved in metabolic or catabolic biochemical pathways, cell division, reproduction, energy metabolism, embryonic development, larval development, transcriptional regulation, and the like. As described herein, ingestion of compositions by a target organism containing one or more dsRNAs, at least one segment of which is specifically complementary to at least a substantially identical segment of RNA produced in the cells of the target pest organism, can result in failure or reduction of the capacity to mate, oviposit, or produce viable progeny. A polynucleotide, either DNA or RNA, derived from a coleopteran pest can be used to construct plant cells resistant to infestation by the pests. The host plant of the coleopteran pest (e.g., Z. mays), for example, can be transformed to contain one or more of the polynucleotides derived from the coleopteran pest as provided herein. The polynucleotide transformed into the host may encode one or more RNAs that form into a dsRNA structure in the cells or biological fluids within the transformed host, thus making the dsRNA available if/when the pest forms a nutritional relationship with the transgenic host. This may result in the suppression of expression of one or more genes in the cells of the pest, and ultimately inhibition of reproduction and/or development.

Thus, in some embodiments, a gene is targeted that is essentially involved in the growth, development and reproduction of a coleopteran pest. Other target genes for use in the present invention may include, for example, those that play important roles in coleopteran pest viability, movement, migration, growth, development, infectivity, and establishment of feeding sites. A target gene may therefore be a housekeeping gene or a transcription factor. Additionally, a native coleopteran pest polynucleotide for use in the present invention may also be derived from a homolog (e.g., an ortholog), of a plant, viral, bacterial or insect gene, the function of which is known to those of skill in the art, and the polynucleotide of which is specifically hybridizable with a target gene in the genome of the target coleopteran pest. Methods for identifying a homolog of a gene with a known nucleotide sequence by hybridization are known to those of skill in the art.

In some embodiments, the invention provides methods for obtaining a nucleic acid molecule comprising a polynucleotide for producing an iRNA (e.g., dsRNA, siRNA, miRNA, shRNA, and hpRNA) molecule. One such embodiment comprises: (a) analyzing one or more target gene(s) for their expression, function, and phenotype upon dsRNA-mediated gene suppression in a coleopteran pest; (b) probing a cDNA or gDNA library with a probe comprising all or a portion of a polynucleotide or a homolog thereof from a targeted coleopteran pest that displays an altered (e.g., reduced) reproduction or development phenotype in a dsRNA-mediated suppression analysis; (c) identifying a DNA clone that specifically hybridizes with the probe; (d) isolating the DNA clone identified in step (b); (e) sequencing the cDNA or gDNA fragment that comprises the clone isolated in step (d), wherein the sequenced nucleic acid molecule comprises all or a substantial portion of the RNA or a homolog thereof; and (f) chemically synthesizing all or a substantial portion of a gene, or an siRNA, miRNA, hpRNA, mRNA, shRNA, or dsRNA.

In further embodiments, a method for obtaining a nucleic acid fragment comprising a polynucleotide for producing a substantial portion of an iRNA (e.g., dsRNA, siRNA, miRNA, shRNA, and hpRNA) molecule includes: (a) synthesizing first and second oligonucleotide primers specifically complementary to a portion of a native polynucleotide from a targeted coleopteran pest; and (b) amplifying a cDNA or gDNA insert present in a cloning vector using the first and second oligonucleotide primers of step (a), wherein the amplified nucleic acid molecule comprises a substantial portion of a siRNA, miRNA, hpRNA, mRNA, shRNA, or dsRNA molecule.

Nucleic acids of the invention can be isolated, amplified, or produced by a number of approaches. For example, an iRNA (e.g., dsRNA, siRNA, miRNA, shRNA, and hpRNA) molecule may be obtained by PCR amplification of a target polynucleotide (e.g., a target gene or a target transcribed non-coding polynucleotide) derived from a gDNA or cDNA library, or portions thereof. DNA or RNA may be extracted from a target organism, and nucleic acid libraries may be prepared therefrom using methods known to those ordinarily skilled in the art. gDNA or cDNA libraries generated from a target organism may be used for PCR amplification and sequencing of target genes. A confirmed PCR product may be used as a template for in vitro transcription to generate sense and antisense RNA with minimal promoters. Alternatively, nucleic acid molecules may be synthesized by any of a number of techniques (See, e.g., Ozaki et al. (1992) Nucleic Acids Research, 20: 5205-5214; and Agrawal et al. (1990) Nucleic Acids Research, 18: 5419-5423), including use of an automated DNA synthesizer (for example, a P.E. Biosystems, Inc. (Foster City, Calif.) model 392 or 394 DNA/RNA Synthesizer), using standard chemistries, such as phosphoramidite chemistry. See, e.g., Beaucage et al. (1992) Tetrahedron, 48: 2223-2311; U.S. Pat. Nos. 4,980,460, 4,725,677, 4,415,732, 4,458,066, and 4,973,679. Alternative chemistries resulting in non-natural backbone groups, such as phosphorothioate, phosphoramidate, and the like, can also be employed.

An RNA, dsRNA, siRNA, miRNA, shRNA, or hpRNA molecule of the present invention may be produced chemically or enzymatically by one skilled in the art through manual or automated reactions, or in vivo in a cell comprising a nucleic acid molecule comprising a polynucleotide encoding the RNA, dsRNA, siRNA, miRNA, shRNA, or hpRNA molecule. RNA may also be produced by partial or total organic synthesis; any modified ribonucleotide can be introduced by in vitro enzymatic or organic synthesis. An RNA molecule may be synthesized by a cellular RNA polymerase or a bacteriophage RNA polymerase (e.g., T3 RNA polymerase, T7 RNA polymerase, and SP6 RNA polymerase). Expression constructs useful for the cloning and expression of polynucleotides are known in the art. See, e.g., International PCT Publication No. WO97/32016; and U.S. Pat. Nos. 5,593,874, 5,698,425, 5,712,135, 5,789,214, and 5,804,693. RNA molecules that are synthesized chemically or by in vitro enzymatic synthesis may be purified prior to introduction into a cell. For example, RNA molecules can be purified from a mixture by extraction with a solvent or resin, precipitation, electrophoresis, chromatography, or a combination thereof. Alternatively, RNA molecules that are synthesized chemically or by in vitro enzymatic synthesis may be used with no or a minimum of purification, for example, to avoid losses due to sample processing. The RNA molecules may be dried for storage or dissolved in an aqueous solution. The solution may contain buffers or salts to promote annealing, and/or stabilization of dsRNA molecule duplex strands.

In particular embodiments, a dsRNA molecule may be formed by a single self-complementary RNA strand or from two complementary RNA strands. dsRNA molecules may be synthesized either in vivo or in vitro. An endogenous RNA polymerase of the cell may mediate transcription of the one or two RNA strands in vivo, or cloned RNA polymerase may be used to mediate transcription in vivo or in vitro. Post-transcriptional inhibition of a target gene in a coleopteran pest may be host-targeted by specific transcription in an organ, tissue, or cell type of the host (e.g., by using a tissue-specific promoter); stimulation of an environmental condition in the host (e.g., by using an inducible promoter that is responsive to infection, stress, temperature, and/or chemical inducers); and/or engineering transcription at a developmental stage or age of the host (e.g., by using a developmental stage-specific promoter). RNA strands that form a dsRNA molecule, whether transcribed in vitro or in vivo, may or may not be polyadenylated, and may or may not be capable of being translated into a polypeptide by a cell's translational apparatus.

### D. Recombinant Vectors and Host Cell Transformation

In some embodiments, the invention also provides a DNA molecule for introduction into a cell (e.g., a bacterial cell, a yeast cell, or a plant cell), wherein the DNA molecule comprises a polynucleotide that, upon expression to RNA and ingestion by a coleopteran pest, achieves suppression of a target gene in a cell, tissue, or organ of the pest. Thus, some embodiments provide a recombinant nucleic acid molecule comprising a polynucleotide capable of being expressed as an iRNA (e.g., dsRNA, siRNA, miRNA, shRNA, and hpRNA) molecule in a plant cell to inhibit target gene expression in a coleopteran pest. In order to initiate or enhance expression, such recombinant nucleic acid molecules may comprise one or more regulatory elements, which regulatory elements may be operably linked to the polynucleotide capable of being expressed as an iRNA. Methods to express a gene suppression molecule in plants are known, and may be used to express a polynucleotide of the present invention. See, e.g., International PCT Publication No. WO06/073727; and U.S. Patent Publication No. 2006/0200878 A1).

In specific embodiments, a recombinant DNA molecule of the invention may comprise a polynucleotide encoding an RNA that may form a dsRNA molecule. Such recombinant DNA molecules may encode RNAs that may form dsRNA molecules capable of inhibiting the expression of endogenous target gene(s) in a coleopteran pest cell upon ingestion. In many embodiments, a transcribed RNA may form a dsRNA molecule that may be provided in a stabilized form; e.g., as a hairpin and stem and loop structure.

In some embodiments, one strand of a dsRNA molecule may be formed by transcription from a polynucleotide which is substantially homologous to the RNA encoded by a polynucleotide selected from the group consisting of SEQ ID NOs:1, 2, and 4; the complement of SEQ ID NOs:1, 2, and/or 4; a fragment of at least 15 contiguous nucleotides of SEQ ID NO:1, SEQ ID NO:2, and/or SEQ ID NO:4; the complement of a fragment of at least 15 contiguous nucleotides of SEQ ID NO:1, SEQ ID NO:2, and/or SEQ ID NO:4; a native coding polynucleotide of a Diabrotica organism (e.g., WCR) comprising SEQ ID NOs:1, 2, and/or 4; the complement of a native coding polynucleotide of a Diabrotica organism comprising SEQ ID NOs:1, 2, and/or 4; a fragment of at least 15 contiguous nucleotides of a native coding polynucleotide of a Diabrotica organism comprising SEQ ID NOs:1, 2, and/or 4; and the complement of a fragment of at least 15 contiguous nucleotides of a native coding polynucleotide of a Diabrotica organism comprising SEQ ID NOs:1, 2, and/or 4.

In particular embodiments, a recombinant DNA molecule encoding an RNA that may form a dsRNA molecule may comprise a coding region wherein at least two polynucleotides are arranged such that one polynucleotide is in a sense orientation, and the other polynucleotide is in an antisense orientation, relative to at least one promoter, wherein the sense polynucleotide and the antisense polynucleotide are linked or connected by a linker of, for example, from about five (˜5) to about one thousand (˜1000) nucleotides. The linker may form a loop between the sense and antisense polynucleotides. The sense polynucleotide or the antisense polynucleotide may be substantially homologous to an RNA encoded by a target gene (e.g., a kruppel gene comprising SEQ ID NO:1 and/or SEQ ID NO:2) or fragment thereof. In some embodiments, however, a recombinant DNA molecule may encode an RNA that may form a dsRNA molecule without a linker. In embodiments, a sense coding polynucleotide and an antisense coding polynucleotide may be different lengths.

Polynucleotides identified as having a deleterious effect on coleopteran pests or a plant-protective effect with regard to coleopteran pests may be readily incorporated into expressed dsRNA molecules through the creation of appropriate expression cassettes in a recombinant nucleic acid molecule of the invention. For example, such polynucleotides may be expressed as a hairpin with stem and loop structure by taking a first segment corresponding to an RNA encoded by a target gene polynucleotide (e.g., a kruppel gene comprising SEQ ID NO:1 and/or SEQ ID NO:2, and fragments thereof (e.g., SEQ ID NO:4)); linking this polynucleotide to a second segment linker region that is not homologous or complementary to the first segment; and linking this to a third segment, wherein at least a portion of the third segment is substantially complementary to the first segment. Such a construct forms a stem and loop structure by intramolecular base-pairing of the first segment with the third segment, wherein the loop structure forms comprising the second segment. See, e.g., U.S. Patent Publication Nos. 2002/0048814 and 2003/0018993; and International PCT Publication Nos. WO94/01550 and WO98/05770. A dsRNA molecule may be generated, for example, in the form of a double-stranded structure such as a stem-loop structure (e.g., hairpin), whereby production of siRNA targeted for a native coleopteran pest polynucleotide is enhanced by co-expression of a fragment of the targeted gene, for instance on an additional plant expressible cassette, that leads to enhanced siRNA production, or reduces methylation to prevent transcriptional gene silencing of the dsRNA hairpin promoter.

Certain embodiments of the invention include introduction of a recombinant nucleic acid molecule of the present invention into a plant (i.e., transformation) to achieve coleopteran pest-protective levels of expression of one or more iRNA molecules. A recombinant DNA molecule may, for example, be a vector, such as a linear or a closed circular plasmid. The vector system may be a single vector or plasmid, or two or more vectors or plasmids that together contain the total DNA to be introduced into the genome of a host. In addition, a vector may be an expression vector. Nucleic acids of the invention can, for example, be suitably inserted into a vector under the control of a suitable promoter that functions in one or more hosts to drive expression of a linked coding polynucleotide or other DNA element. Many vectors are available for this purpose, and selection of the appropriate vector will depend mainly on the size of the nucleic acid to be inserted into the vector and the particular host cell to be transformed with the vector. Each vector contains various components depending on its function (e.g., amplification of DNA or expression of DNA) and the particular host cell with which it is compatible.

To impart protection from a coleopteran pest to a transgenic plant, a recombinant DNA may, for example, be transcribed into an iRNA molecule (e.g., an RNA molecule that forms a dsRNA molecule) within the tissues or fluids of the recombinant plant. An iRNA molecule may comprise a polynucleotide that is substantially homologous and specifically hybridizable to a corresponding transcribed polynucleotide within a coleopteran pest that may cause damage to the host plant species. The coleopteran pest may contact the iRNA molecule that is transcribed in cells of the transgenic host plant, for example, by ingesting cells or fluids of the transgenic host plant that comprise the iRNA molecule. Thus, expression of a target gene is suppressed by the iRNA molecule within coleopteran pests that infest the transgenic host plant. In some embodiments, suppression of expression of the target gene in the target coleopteran pest may result in the plant being resistant to attack by the pest.

In order to enable delivery of iRNA molecules to a coleopteran pest in a nutritional relationship with a plant cell that has been transformed with a recombinant nucleic acid molecule of the invention, expression (i.e., transcription) of iRNA molecules in the plant cell is required. Thus, a recombinant nucleic acid molecule may comprise a polynucleotide of the invention operably linked to one or more regulatory elements, such as a heterologous promoter element that functions in a host cell, such as a bacterial cell wherein the nucleic acid molecule is to be amplified, and a plant cell wherein the nucleic acid molecule is to be expressed.

Promoters suitable for use in nucleic acid molecules of the invention include those that are inducible, viral, synthetic, or constitutive, all of which are well known in the art. Non-limiting examples describing such promoters include U.S. Pat. No. 6,437,217 (maize RS81 promoter); U.S. Pat. No. 5,641,876 (rice actin promoter); U.S. Pat. No. 6,426,446 (maize RS324 promoter); U.S. Pat. No. 6,429,362 (maize PR-1 promoter); U.S. Pat. No. 6,232,526 (maize A3 promoter); U.S. Pat. No. 6,177,611 (constitutive maize promoters); U.S. Pat. Nos. 5,322,938, 5,352,605, 5,359,142, and 5,530,196 (CaMV 35S promoter); U.S. Pat. No. 6,433,252 (maize L3 oleosin promoter); U.S. Pat. No. 6,429,357 (rice actin 2 promoter, and rice actin 2 intron); U.S. Pat. No. 6,294,714 (light-inducible promoters); U.S. Pat. No. 6,140,078 (salt-inducible promoters); U.S. Pat. No. 6,252,138 (pathogen-inducible promoters); U.S. Pat. No. 6,175,060 (phosphorous deficiency-inducible promoters); U.S. Pat. No. 6,388,170 (bidirectional promoters); U.S. Pat. No. 6,635,806 (gamma-coixin promoter); and U.S. Patent Publication No. 2009/757,089 (maize chloroplast aldolase promoter). Additional promoters include the nopaline synthase (NOS) promoter (Ebert et al. (1987) Proc. Natl. Acad. Sci. USA 84(16):5745-9) and the octopine synthase (OCS) promoters (which are carried on tumor-inducing plasmids of Agrobacterium tumefaciens); the caulimovirus promoters such as the cauliflower mosaic virus (CaMV) 19S promoter (Lawton et al. (1987) Plant Mol. Biol. 9:315-24); the CaMV 35S promoter (Odell et al. (1985) Nature 313:810-2; the figwort mosaic virus 35S-promoter (Walker et al. (1987) Proc. Natl. Acad. Sci. USA 84(19):6624-8); the sucrose synthase promoter (Yang and Russell (1990) Proc. Natl. Acad. Sci. USA 87:4144-8); the R gene complex promoter (Chandler et al. (1989) Plant Cell 1:1175-83); the chlorophyll a/b binding protein gene promoter; CaMV 35S (U.S. Pat. Nos. 5,322,938, 5,352,605, 5,359,142, and 5,530,196); FMV 35S (U.S. Pat. Nos. 6,051,753, and 5,378,619); a PC1SV promoter (U.S. Pat. No. 5,850,019); the SCP1 promoter (U.S. Pat. No. 6,677,503); and AGRtu.nos promoters (GenBank™ Accession No. V00087; Depicker et al. (1982) J. Mol. Appl. Genet. 1:561-73; Bevan et al. (1983) Nature 304:184-7).

In particular embodiments, nucleic acid molecules of the invention comprise a tissue-specific promoter, such as a root-specific promoter. Root-specific promoters drive expression of operably-linked coding polynucleotides exclusively or preferentially in root tissue. Examples of root-specific promoters are known in the art. See, e.g., U.S. Pat. Nos. 5,110,732; 5,459,252 and 5,837,848; and Opperman et al. (1994) Science 263:221-3; and Hirel et al. (1992) Plant Mol. Biol. 20:207-18. In some embodiments, a polynucleotide or fragment for coleopteran pest control according to the invention may be cloned between two root-specific promoters oriented in opposite transcriptional directions relative to the polynucleotide or fragment, and which are operable in a transgenic plant cell and expressed therein to produce RNA molecules in the transgenic plant cell that subsequently may form dsRNA molecules, as described, supra. The iRNA molecules expressed in plant tissues may be ingested by a coleopteran pest so that suppression of target gene expression is achieved.

Additional regulatory elements that may optionally be operably linked to a nucleic acid molecule of interest include 5′UTRs located between a promoter element and a coding polynucleotide that function as a translation leader element. The translation leader element is present in the fully-processed mRNA, and it may affect processing of the primary transcript, and/or RNA stability. Examples of translation leader elements include maize and petunia heat shock protein leaders (U.S. Pat. No. 5,362,865), plant virus coat protein leaders, plant rubisco leaders, and others. See, e.g., Turner and Foster (1995) Molecular Biotech. 3(3):225-36. Non-limiting examples of 5′UTRs include GmHsp (U.S. Pat. No. 5,659,122); PhDnaK (U.S. Pat. No. 5,362,865); AtAnt1; TEV (Carrington and Freed (1990) J. Virol. 64:1590-7); and AGRtunos (GenBank™ Accession No. V00087; and Bevan et al. (1983) Nature 304:184-7).

Additional regulatory elements that may optionally be operably linked to a nucleic acid molecule of interest also include 3′ non-translated elements, 3′ transcription termination regions, or polyadenylation regions. These are genetic elements located downstream of a polynucleotide, and include polynucleotides that provide polyadenylation signal, and/or other regulatory signals capable of affecting transcription or mRNA processing. The polyadenylation signal functions in plants to cause the addition of polyadenylate nucleotides to the 3′ end of the mRNA precursor. The polyadenylation element can be derived from a variety of plant genes, or from T-DNA genes. A non-limiting example of a 3′ transcription termination region is the nopaline synthase 3′ region (nos 3; Fraley et al. (1983) Proc. Natl. Acad. Sci. USA 80:4803-7). An example of the use of different 3′ nontranslated regions is provided in Ingelbrecht et al., (1989) Plant Cell 1:671-80. Non-limiting examples of polyadenylation signals include one from a Pisum sativum RbcS2 gene (Ps.RbcS2-E9; Coruzzi et al. (1984) EMBO J. 3:1671-9) and AGRtu.nos (GenBank™ Accession No. E01312).

Some embodiments may include a plant transformation vector that comprises an isolated and purified DNA molecule comprising at least one of the above-described regulatory elements operatively linked to one or more polynucleotides of the present invention. When expressed, the one or more polynucleotides result in one or more RNA molecule(s) comprising a polynucleotide that is specifically complementary to all or part of a native RNA molecule in a coleopteran pest. Thus, the polynucleotide(s) may comprise a segment encoding all or part of a polyribonucleotide present within a targeted coleopteran pest RNA transcript, and may comprise inverted repeats of all or a part of a targeted pest transcript. A plant transformation vector may contain polynucleotides specifically complementary to more than one target polynucleotide, thus allowing production of more than one dsRNA for inhibiting expression of two or more genes in cells of one or more populations or species of target coleopteran pests. Segments of polynucleotides specifically complementary to polynucleotides present in different genes can be combined into a single composite nucleic acid molecule for expression in a transgenic plant. Such segments may be contiguous or separated by a linker.

In other embodiments, a plasmid of the present invention already containing at least one polynucleotide(s) of the invention can be modified by the sequential insertion of additional polynucleotide(s) in the same plasmid, wherein the additional polynucleotide(s) are operably linked to the same regulatory elements as the original at least one polynucleotide(s). In some embodiments, a nucleic acid molecule may be designed for the inhibition of multiple target genes. In some embodiments, the multiple genes to be inhibited can be obtained from the same coleopteran pest species, which may enhance the effectiveness of the nucleic acid molecule. In other embodiments, the genes can be derived from different insect (e.g., coleopteran) pests, which may broaden the range of pests against which the agent(s) is/are effective. When multiple genes are targeted for suppression or a combination of expression and suppression, a polycistronic DNA element can be engineered.

A recombinant nucleic acid molecule or vector of the present invention may comprise a selectable marker that confers a selectable phenotype on a transformed cell, such as a plant cell. Selectable markers may also be used to select for plants or plant cells that comprise a recombinant nucleic acid molecule of the invention. The marker may encode biocide resistance, antibiotic resistance (e.g., kanamycin, Geneticin (G418), bleomycin, hygromycin, etc.), or herbicide tolerance (e.g., glyphosate, etc.). Examples of selectable markers include, but are not limited to: a neo gene which codes for kanamycin resistance and can be selected for using kanamycin, G418, etc.; a bar gene which codes for bialaphos resistance; a mutant EPSP synthase gene which encodes glyphosate tolerance; a nitrilase gene which confers resistance to bromoxynil; a mutant acetolactate synthase (ALS) gene which confers imidazolinone or sulfonylurea tolerance; and a methotrexate resistant DHFR gene. Multiple selectable markers are available that confer resistance to ampicillin, bleomycin, chloramphenicol, gentamycin, hygromycin, kanamycin, lincomycin, methotrexate, phosphinothricin, puromycin, spectinomycin, rifampicin, streptomycin and tetracycline, and the like. Examples of such selectable markers are illustrated in, e.g., U.S. Pat. Nos. 5,550,318; 5,633,435; 5,780,708; and 6,118,047.

A recombinant nucleic acid molecule or vector of the present invention may also include a screenable marker. Screenable markers may be used to monitor expression. Exemplary screenable markers include a β-glucuronidase or uidA gene (GUS) which encodes an enzyme for which various chromogenic substrates are known (Jefferson et al. (1987) Plant Mol. Biol. Rep. 5:387-405); an R-locus gene, which encodes a product that regulates the production of anthocyanin pigments (red color) in plant tissues (Dellaporta et al. (1988) “Molecular cloning of the maize R-nj allele by transposon tagging with Ac.” In 18^{th} Stadler Genetics Symposium, P. Gustafson and R. Appels, eds. (New York: Plenum), pp. 263-82); a β-lactamase gene (Sutcliffe et al. (1978) Proc. Natl. Acad. Sci. USA 75:3737-41); a gene which encodes an enzyme for which various chromogenic substrates are known (e.g., PADAC, a chromogenic cephalosporin); a luciferase gene (Ow et al. (1986) Science 234:856-9); an xy/E gene that encodes a catechol dioxygenase that can convert chromogenic catechols (Zukowski et al. (1983) Gene 46(2-3):247-55); an amylase gene (Ikatu et al. (1990) Bio/Technol. 8:241-2); a tyrosinase gene which encodes an enzyme capable of oxidizing tyrosine to DOPA and dopaquinone which in turn condenses to melanin (Katz et al. (1983) J. Gen. Microbiol. 129:2703-14); and an α-galactosidase.

In some embodiments, recombinant nucleic acid molecules, as described, supra, may be used in methods for the creation of transgenic plants and expression of heterologous nucleic acids in plants to prepare transgenic plants that exhibit reduced susceptibility to coleopteran pests. Plant transformation vectors can be prepared, for example, by inserting nucleic acid molecules encoding iRNA molecules into plant transformation vectors and introducing these into plants.

Suitable methods for transformation of host cells include any method by which DNA can be introduced into a cell, such as by transformation of protoplasts (See, e.g., U.S. Pat. No. 5,508,184), by desiccation/inhibition-mediated DNA uptake (See, e.g., Potrykus et al. (1985) Mol. Gen. Genet. 199:183-8), by electroporation (See, e.g., U.S. Pat. No. 5,384,253), by agitation with silicon carbide fibers (See, e.g., U.S. Pat. Nos. 5,302,523 and 5,464,765), by Agrobacterium-mediated transformation (See, e.g., U.S. Pat. Nos. 5,563,055; 5,591,616; 5,693,512; 5,824,877; 5,981,840; and 6,384,301) and by acceleration of DNA-coated particles (See, e.g., U.S. Pat. Nos. 5,015,580; 5,550,318; 5,538,880; 6,160,208; 6,399,861; and 6,403,865), etc. Techniques that are particularly useful for transforming corn are described, for example, in U.S. Pat. Nos. 7,060,876 and 5,591,616; and International PCT Publication WO95/06722. Through the application of techniques such as these, the cells of virtually any species may be stably transformed. In some embodiments, transforming DNA is integrated into the genome of the host cell. In the case of multicellular species, transgenic cells may be regenerated into a transgenic organism. Any of these techniques may be used to produce a transgenic plant, for example, comprising one or more nucleic acids encoding one or more iRNA molecules in the genome of the transgenic plant.

The most widely utilized method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. The Ti (tumor-inducing)-plasmids contain a large segment, known as T-DNA, which is transferred to transformed plants. Another segment of the Ti plasmid, the Vir region, is responsible for T-DNA transfer. The T-DNA region is bordered by terminal repeats. In modified binary vectors, the tumor-inducing genes have been deleted, and the functions of the Vir region are utilized to transfer foreign DNA bordered by the T-DNA border elements. The T-region may also contain a selectable marker for efficient recovery of transgenic cells and plants, and a multiple cloning site for inserting polynucleotides for transfer such as a dsRNA encoding nucleic acid.

Thus, in some embodiments, a plant transformation vector is derived from a Ti plasmid of A. tumefaciens (See, e.g., U.S. Pat. Nos. 4,536,475, 4,693,977, 4,886,937, and 5,501,967; and European Patent No. EP 0 122 791) or a Ri plasmid of A. rhizogenes. Additional plant transformation vectors include, for example and without limitation, those described by Herrera-Estrella et al. (1983) Nature 303:209-13; Bevan et al. (1983) Nature 304:184-7; Klee et al. (1985) Bio/Technol. 3:637-42; and in European Patent No. EP 0 120 516, and those derived from any of the foregoing. Other bacteria such as Sinorhizobium, Rhizobium, and Mesorhizobium that interact with plants naturally can be modified to mediate gene transfer to a number of diverse plants. These plant-associated symbiotic bacteria can be made competent for gene transfer by acquisition of both a disarmed Ti plasmid and a suitable binary vector.

After providing exogenous DNA to recipient cells, transformed cells are generally identified for further culturing and plant regeneration. In order to improve the ability to identify transformed cells, one may desire to employ a selectable or screenable marker gene, as previously set forth, with the transformation vector used to generate the transformant. In the case where a selectable marker is used, transformed cells are identified within the potentially transformed cell population by exposing the cells to a selective agent or agents. In the case where a screenable marker is used, cells may be screened for the desired marker gene trait.

Cells that survive the exposure to the selective agent, or cells that have been scored positive in a screening assay, may be cultured in media that supports regeneration of plants. In some embodiments, any suitable plant tissue culture media (e.g., MS and N6 media) may be modified by including further substances, such as growth regulators. Tissue may be maintained on a basic medium with growth regulators until sufficient tissue is available to begin plant regeneration efforts, or following repeated rounds of manual selection, until the morphology of the tissue is suitable for regeneration (e.g., at least 2 weeks), then transferred to media conducive to shoot formation. Cultures are transferred periodically until sufficient shoot formation has occurred. Once shoots are formed, they are transferred to media conducive to root formation. Once sufficient roots are formed, plants can be transferred to soil for further growth and maturation.

To confirm the presence of a nucleic acid molecule of interest (for example, a DNA encoding one or more iRNA molecules that inhibit target gene expression in a coleopteran pest) in the regenerating plants, a variety of assays may be performed. Such assays include, for example: molecular biological assays, such as Southern and northern blotting, PCR, and nucleic acid sequencing; biochemical assays, such as detecting the presence of a protein product, e.g., by immunological means (ELISA and/or western blots) or by enzymatic function; plant part assays, such as leaf or root assays; and analysis of the phenotype of the whole regenerated plant.

Integration events may be analyzed, for example, by PCR amplification using, e.g., oligonucleotide primers specific for a nucleic acid molecule of interest. PCR genotyping is understood to include, but not be limited to, polymerase-chain reaction (PCR) amplification of gDNA derived from isolated host plant callus tissue predicted to contain a nucleic acid molecule of interest integrated into the genome, followed by standard cloning and sequence analysis of PCR amplification products. Methods of PCR genotyping have been well described (for example, Rios, G. et al. (2002) Plant J. 32:243-53) and may be applied to gDNA derived from any plant species (e.g., Z. mays) or tissue type, including cell cultures.

A transgenic plant formed using Agrobacterium-dependent transformation methods typically contains a single recombinant DNA inserted into one chromosome. The polynucleotide of the single recombinant DNA is referred to as a “transgenic event” or “integration event”. Such transgenic plants are heterozygous for the inserted exogenous polynucleotide. In some embodiments, a transgenic plant homozygous with respect to a transgene may be obtained by sexually mating (selfing) an independent segregant transgenic plant that contains a single exogenous gene to itself, for example a T₀ plant, to produce T₁ seed. One fourth of the T₁ seed produced will be homozygous with respect to the transgene. Germinating T₁ seed results in plants that can be tested for heterozygosity, typically using an SNP assay or a thermal amplification assay that allows for the distinction between heterozygotes and homozygotes (i.e., a zygosity assay).

In particular embodiments, at least 2, 3, 4, 5, 6, 7, 8, 9 or 10 or more different iRNA molecules are produced in a plant cell that have a coleopteran pest-protective effect. The iRNA molecules (e.g., dsRNA molecules) may be expressed from multiple nucleic acids introduced in different transformation events, or from a single nucleic acid introduced in a single transformation event. In some embodiments, a plurality of iRNA molecules are expressed under the control of a single promoter. In other embodiments, a plurality of iRNA molecules are expressed under the control of multiple promoters. Single iRNA molecules may be expressed that comprise multiple polynucleotides that are each homologous to different loci within one or more coleopteran pests (for example, the loci defined by SEQ ID NOs:1, 2, and 4), both in different populations of the same species of coleopteran pest, or in different species of coleopteran pests.

In addition to direct transformation of a plant with a recombinant nucleic acid molecule, transgenic plants can be prepared by crossing a first plant having at least one transgenic event with a second plant lacking such an event. For example, a recombinant nucleic acid molecule comprising a polynucleotide that encodes an iRNA molecule may be introduced into a first plant line that is amenable to transformation to produce a transgenic plant, which transgenic plant may be crossed with a second plant line to introgress the polynucleotide that encodes the iRNA molecule into the second plant line.

The invention also includes commodity products containing one or more of the polynucleotides of the present invention. Particular embodiments include commodity products produced from a recombinant plant or seed containing one or more of the polynucleotides of the present invention. A commodity product containing one or more of the polynucleotides of the present invention is intended to include, but not be limited to, meals, oils, crushed or whole grains or seeds of a plant, or any food product comprising any meal, oil, or crushed or whole grain of a recombinant plant or seed containing one or more of the polynucleotides of the present invention. The detection of one or more of the polynucleotides of the present invention in one or more commodity or commodity products contemplated herein is de facto evidence that the commodity or commodity product is produced from a transgenic plant designed to express one or more of the polynucleotides of the present invention for the purpose of controlling plant pests using dsRNA-mediated gene suppression methods.

In some aspects, seeds and commodity products produced by transgenic plants derived from transformed plant cells are included, wherein the seeds or commodity products comprise a detectable amount of a nucleic acid of the invention. In some embodiments, such commodity products may be produced, for example, by obtaining transgenic plants and preparing food or feed from them. Commodity products comprising one or more of the polynucleotides of the invention includes, for example and without limitation: meals, oils, crushed or whole grains or seeds of a plant, and any food product comprising any meal, oil, or crushed or whole grain of a recombinant plant or seed comprising one or more of the nucleic acids of the invention. The detection of one or more of the polynucleotides of the invention in one or more commodity or commodity products is de facto evidence that the commodity or commodity product is produced from a transgenic plant designed to express one or more of the iRNA molecules of the invention for the purpose of controlling coleopteran pests.

In some embodiments, a transgenic plant or seed comprising a nucleic acid molecule of the invention also may comprise at least one other transgenic event in its genome, including without limitation: a transgenic event from which is transcribed an iRNA molecule targeting a locus in a coleopteran pest other than the ones defined by SEQ ID NOs: 1, 2, and 4; a transgenic event from which is transcribed an iRNA molecule targeting a gene in an organism other than a coleopteran pest (e.g., a plant-parasitic nematode); a gene encoding an insecticidal protein (e.g., a Bacillus thuringiensis insecticidal protein); a herbicide tolerance gene (e.g., a gene providing tolerance to glyphosate); and a gene contributing to a desirable phenotype in the transgenic plant, such as increased yield, altered fatty acid metabolism, or restoration of cytoplasmic male sterility. In particular embodiments, polynucleotides encoding iRNA molecules of the invention may be combined with other insect control and disease traits in a plant to achieve desired traits for enhanced control of plant disease and insect damage. Combining insect control traits that employ distinct modes-of-action may provide protected transgenic plants with superior durability over plants harboring a single control trait, for example, because of the reduced probability that resistance to the trait(s) will develop in the field.

### V. Target Gene Suppression in a Coleopteran Pest

### A. Overview

In some embodiments of the invention, at least one nucleic acid molecule useful for the control of coleopteran pests may be provided to a coleopteran pest, wherein the nucleic acid molecule leads to RNAi-mediated gene silencing in the pest. In particular embodiments, an iRNA molecule (e.g., dsRNA, siRNA, miRNA, shRNA, and hpRNA) may be provided to a pest. In some embodiments, a nucleic acid molecule useful for the control of coleopteran pests may be provided to a pest by contacting the nucleic acid molecule with the pest. In these and further embodiments, a nucleic acid molecule useful for the control of coleopteran pests may be provided in a feeding substrate of the pest, for example, a nutritional composition. In these and further embodiments, a nucleic acid molecule useful for the control of a coleopteran pest may be provided through ingestion of plant material comprising the nucleic acid molecule that is ingested by the pest. In certain embodiments, the nucleic acid molecule is present in plant material through expression of a recombinant nucleic acid introduced into the plant material, for example, by transformation of a plant cell with a vector comprising the recombinant nucleic acid and regeneration of a plant material or whole plant from the transformed plant cell.

### B. RNAi-Mediated Target Gene Suppression

In embodiments, the invention provides iRNA molecules (e.g., dsRNA, siRNA, miRNA, shRNA, and hpRNA) that may be designed to target essential native polynucleotides (e.g., essential genes) in the transcriptome of a coleopteran (e.g., WCR or NCR) pest, for example by designing an iRNA molecule that comprises at least one strand comprising a polynucleotide that is specifically complementary to the target polynucleotide. The sequence of an iRNA molecule so designed may be identical to that of the target polynucleotide, or may incorporate mismatches that do not prevent specific hybridization between the iRNA molecule and its target polynucleotide.

iRNA molecules of the invention may be used in methods for gene suppression in a coleopteran pest, thereby reducing the level or incidence of damage caused by the pest on a plant (for example, a protected transformed plant comprising an iRNA molecule). As used herein the term “gene suppression” refers to any of the well-known methods for reducing the levels of protein produced as a result of gene transcription to mRNA and subsequent translation of the mRNA, including the reduction of protein expression from a gene or a coding polynucleotide including post-transcriptional inhibition of expression and transcriptional suppression. Post-transcriptional inhibition is mediated by specific homology between all or a part of an mRNA transcribed from a gene targeted for suppression and the corresponding iRNA molecule used for suppression. Additionally, post-transcriptional inhibition refers to the substantial and measurable reduction of the amount of mRNA available in the cell for binding by ribosomes.

In particular embodiments wherein an iRNA molecule is a dsRNA molecule, the dsRNA molecule may be cleaved by the enzyme, DICER, into short siRNA molecules (approximately 20 nucleotides in length). The double-stranded siRNA molecule generated by DICER activity upon the dsRNA molecule may be separated into two single-stranded siRNAs; the “passenger strand” and the “guide strand”. The passenger strand may be degraded, and the guide strand may be incorporated into RISC. Post-transcriptional inhibition occurs by specific hybridization of the guide strand with a specifically complementary polynucleotide of an mRNA molecule, and subsequent cleavage by the enzyme, Argonaute (catalytic component of the RISC complex).

In other embodiments of the invention, any form of iRNA molecule may be used. Those of skill in the art will understand that dsRNA molecules typically are more stable during preparation and during the step of providing the iRNA molecule to a cell than are single-stranded RNA molecules, and are typically also more stable in a cell. Thus, while siRNA and miRNA molecules, for example, may be equally effective in some embodiments, a dsRNA molecule may be chosen due to its stability.

In certain embodiments, a nucleic acid molecule is provided that comprises a polynucleotide, which polynucleotide may be expressed in vitro to produce an iRNA molecule that is substantially homologous to a nucleic acid molecule encoded by a polynucleotide within the genome of a coleopteran pest. In certain embodiments, the in vitro transcribed iRNA molecule may be a stabilized dsRNA molecule that comprises a stem-loop structure. After a coleopteran pest contacts the in vitro transcribed iRNA molecule, post-transcriptional inhibition of a target gene in the pest (for example, an essential gene) may occur.

In some embodiments of the invention, expression of an iRNA from a nucleic acid molecule comprising at least 15 contiguous nucleotides (e.g., at least 19 contiguous nucleotides) of a polynucleotide are used in a method for post-transcriptional inhibition of a target gene in a coleopteran pest, wherein the polynucleotide is selected from the group consisting of: SEQ ID NO:1; the complement of SEQ ID NO:1; SEQ ID NO:2; the complement of SEQ ID NO:2; SEQ ID NO:4; the complement of SEQ ID NO:4; a fragment of at least 15 contiguous nucleotides of SEQ ID NO:1; the complement of a fragment of at least 15 contiguous nucleotides of SEQ ID NO:1; a fragment of at least 15 contiguous nucleotides of SEQ ID NO:2; the complement of a fragment of at least 15 contiguous nucleotides of SEQ ID NO:2; a native coding polynucleotide of a Diabrotica organism comprising SEQ ID NO:1; the complement of a native coding polynucleotide of a Diabrotica organism comprising SEQ ID NO:1; a native coding polynucleotide of a Diabrotica organism comprising SEQ ID NO:2; the complement of a native coding polynucleotide of a Diabrotica organism comprising SEQ ID NO:2; a fragment of at least 15 contiguous nucleotides of a native coding polynucleotide of a Diabrotica organism comprising SEQ ID NO:1; the complement of a fragment of at least 15 contiguous nucleotides of a native coding polynucleotide of a Diabrotica organism comprising SEQ ID NO:1; a fragment of at least 15 contiguous nucleotides of a native coding polynucleotide of a Diabrotica organism comprising SEQ ID NO:2; and the complement of a fragment of at least 15 contiguous nucleotides of a native coding polynucleotide of a Diabrotica organism comprising SEQ ID NO:2. In certain embodiments, expression of a nucleic acid molecule that is at least about 80% identical (e.g., 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, and 100%) with any of the foregoing may be used. In these and further embodiments, a nucleic acid molecule may be expressed that specifically hybridizes to an RNA molecule present in at least one cell of a coleopteran pest.

It is an important feature of some embodiments herein that the RNAi post-transcriptional inhibition system is able to tolerate sequence variations among target genes that might be expected due to genetic mutation, strain polymorphism, or evolutionary divergence. The introduced nucleic acid molecule may not need to be absolutely homologous to either a primary transcription product or a fully processed mRNA of a target gene, so long as the introduced nucleic acid molecule is specifically hybridizable to either a primary transcription product or a fully-processed mRNA of the target gene. Moreover, the introduced nucleic acid molecule may not need to be full-length, relative to either a primary transcription product or a fully processed mRNA of the target gene.

Inhibition of a target gene using the iRNA technology of the present invention is sequence-specific; i.e., polynucleotides substantially homologous to the iRNA molecule(s) are targeted for genetic inhibition. In some embodiments, an RNA molecule comprising a polynucleotide with a nucleotide sequence that is identical to that of a portion of a target gene may be used for inhibition. In these and further embodiments, an RNA molecule comprising a polynucleotide with one or more insertion, deletion, and/or point mutations relative to a target polynucleotide may be used. In particular embodiments, an iRNA molecule and a portion of a target gene may share, for example, at least from about 80%, at least from about 81%, at least from about 82%, at least from about 83%, at least from about 84%, at least from about 85%, at least from about 86%, at least from about 87%, at least from about 88%, at least from about 89%, at least from about 90%, at least from about 91%, at least from about 92%, at least from about 93%, at least from about 94%, at least from about 95%, at least from about 96%, at least from about 97%, at least from about 98%, at least from about 99%, at least from about 100%, and 100% sequence identity. Alternatively, the duplex region of a dsRNA molecule may be specifically hybridizable with a portion of a target gene transcript. In specifically hybridizable molecules, a less than full length polynucleotide exhibiting a greater homology compensates for a longer, less homologous polynucleotide. The length of the polynucleotide of a duplex region of a dsRNA molecule that is identical to a portion of a target gene transcript may be at least about 25, 50, 100, 200, 300, 400, 500, or at least about 1000 bases. In some embodiments, a polynucleotide of greater than 20-100 nucleotides may be used; for example, a polynucleotide of 100-200 or 300-500 nucleotides may be used. In particular embodiments, a polynucleotide of greater than about 200-300 nucleotides may be used. In particular embodiments, a polynucleotide of greater than about 500-1000 nucleotides may be used, depending on the size of the target gene.

In certain embodiments, expression of a target gene in a coleopteran pest may be inhibited by at least 10%; at least 33%; at least 50%; or at least 80% within a cell of the pest, such that a significant inhibition takes place. Significant inhibition refers to inhibition over a threshold that results in a detectable phenotype (e.g., cessation of reproduction, feeding, development, etc.), or a detectable decrease in RNA and/or gene product corresponding to the target gene being inhibited. Although in certain embodiments of the invention inhibition occurs in substantially all cells of the pest, in other embodiments inhibition occurs only in a subset of cells expressing the target gene.

In some embodiments, transcriptional suppression is mediated by the presence in a cell of a dsRNA molecule exhibiting substantial sequence identity to a promoter DNA or the complement thereof to effect what is referred to as “promoter trans suppression.” Gene suppression may be effective against target genes in a coleopteran pest that may ingest or contact such dsRNA molecules, for example, by ingesting or contacting plant material containing the dsRNA molecules. dsRNA molecules for use in promoter trans suppression may be specifically designed to inhibit or suppress the expression of one or more homologous or complementary polynucleotides in the cells of the coleopteran pest. Post-transcriptional gene suppression by antisense or sense oriented RNA to regulate gene expression in plant cells is disclosed in U.S. Pat. Nos. 5,107,065; 5,759,829; 5,283,184; and 5,231,020.

### C. Expression of iRNA Molecules Provided to a Coleopteran Pest

Expression of iRNA molecules for RNAi-mediated gene inhibition in a coleopteran pest may be carried out in any one of many in vitro or in vivo formats. The iRNA molecules may then be provided to a coleopteran pest, for example, by contacting the iRNA molecules with the pest, or by causing the pest to ingest or otherwise internalize the iRNA molecules. Some embodiments of the invention include transformed host plants of a coleopteran pest, transformed plant cells, and progeny of transformed plants. The transformed plant cells and transformed plants may be engineered to express one or more of the iRNA molecules, for example, under the control of a heterologous promoter, to provide a pest-protective effect. Thus, when a transgenic plant or plant cell is consumed by a coleopteran pest during feeding, the pest may ingest iRNA molecules expressed in the transgenic plants or cells. The polynucleotides of the present invention may also be introduced into a wide variety of prokaryotic and eukaryotic microorganism hosts to produce iRNA molecules. The term “microorganism” includes prokaryotic and eukaryotic species, such as bacteria and fungi.

Modulation of gene expression may include partial or complete suppression of such expression. In another embodiment, a method for suppression of gene expression in a coleopteran pest comprises providing in the tissue of the host of the pest a gene-suppressive amount of at least one dsRNA molecule formed following transcription of a polynucleotide as described herein, at least one segment of which is complementary to an mRNA within the cells of the coleopteran pest. A dsRNA molecule, including its modified form such as an siRNA, miRNA, shRNA, or hpRNA molecule, ingested by a coleopteran pest in accordance with the invention may be at least from about 80%, 81%, 82%, 83%, 84%, 85%, 86%, 87%, 88%, 89%, 90%, 91%, 92%, 93%, 94%, 95%, 96%, 97%, 98%, 99%, or about 100% identical to an RNA molecule transcribed from a kruppel DNA molecule, for example, comprising a polynucleotide selected from the group consisting of SEQ ID NOs:1, 2, and 4. Isolated and substantially purified nucleic acid molecules including, but not limited to, non-naturally occurring polynucleotides and recombinant DNA constructs for providing dsRNA molecules of the present invention are therefore provided, which suppress or inhibit the expression of an endogenous coding polynucleotide or a target coding polynucleotide in the coleopteran pest when introduced thereto.

Particular embodiments provide a delivery system for the delivery of iRNA molecules for the post-transcriptional inhibition of one or more target gene(s) in a coleopteran plant pest and control of a population of the plant pest. In some embodiments, the delivery system comprises ingestion of a host transgenic plant cell or contents of the host cell comprising RNA molecules transcribed in the host cell. In these and further embodiments, a transgenic plant cell or a transgenic plant is created that contains a recombinant DNA construct providing a stabilized dsRNA molecule of the invention. Transgenic plant cells and transgenic plants comprising nucleic acids encoding a particular iRNA molecule may be produced by employing recombinant DNA technologies (which basic technologies are well-known in the art) to construct a plant transformation vector comprising a polynucleotide encoding an iRNA molecule of the invention (e.g., a stabilized dsRNA molecule); to transform a plant cell or plant; and to generate the transgenic plant cell or the transgenic plant that contains the transcribed iRNA molecule.

To impart protection from coleopteran pests to a transgenic plant, a recombinant DNA molecule may, for example, be transcribed into an iRNA molecule, such as a dsRNA molecule, an siRNA molecule, an miRNA molecule, an shRNA molecule, or an hpRNA molecule. In some embodiments, an RNA molecule transcribed from a recombinant DNA molecule may form a dsRNA molecule within the tissues or fluids of the recombinant plant. Such a dsRNA molecule may be comprised in part of a polynucleotide that is identical to a corresponding polynucleotide transcribed from a DNA within a coleopteran pest of a type that may infest the host plant. Expression of a target gene within the coleopteran pest is suppressed by the dsRNA molecule, and the suppression of expression of the target gene in the coleopteran pest results in the transgenic plant being resistant to the pest. The modulatory effects of dsRNA molecules have been shown to be applicable to a variety of genes expressed in pests, including, for example, endogenous genes responsible for cell division, chromosomal remodeling, and cellular metabolism or cellular transformation, including housekeeping genes; transcription factors; molting-related genes; and other genes which encode polypeptides involved in cellular metabolism or normal growth and development.

For transcription from a transgene in vivo or an expression construct, a regulatory region (e.g., promoter, enhancer, silencer, and polyadenylation signal) may be used in some embodiments to transcribe the RNA strand (or strands). Therefore, in some embodiments, as set forth, supra, a polynucleotide for use in producing iRNA molecules may be operably linked to one or more promoter elements functional in a plant host cell. The promoter may be an endogenous promoter, normally resident in the host genome. The polynucleotide of the present invention, under the control of an operably linked promoter element, may further be flanked by additional elements that advantageously affect its transcription and/or the stability of a resulting transcript. Such elements may be located upstream of the operably linked promoter, downstream of the 3′ end of the expression construct, and may occur both upstream of the promoter and downstream of the 3′ end of the expression construct.

In some embodiments, suppression of a target gene (e.g., a kruppel gene) results in a parental RNAi phenotype; a phenotype that is observable in progeny of the subject (e.g., a coleopteran pest) contacted with the iRNA molecule. In some embodiments, the pRNAi phenotype comprises the pest being rendered less able to produce viable offspring. In particular examples of pRNAi, a nucleic acid that initiates pRNAi does not increase the incidence of mortality in a population into which the nucleic acid is delivered. In other examples of pRNAi, a nucleic acid that initiates pRNAi also increases the incidence of mortality in a population into which the nucleic acid is delivered.

In other embodiments, a population of coleopteran pests is contacted with an iRNA molecule, thereby resulting in pRNAi, wherein the pests survive and mate but produce eggs that are less able to hatch viable progeny than eggs produced by pests of the same species that are not provided the nucleic acid(s). In some examples, such pests do not oviposit or produce fewer eggs than what is observable in pests of the same species that are not contacted with the iRNA molecule. In some examples, the eggs oviposited by such pests do not hatch or hatch at a rate that is significantly less than what is observable in pests of the same species that are not contacted with the iRNA molecule. In some examples, the larvae that hatch from eggs oviposited by such pests are not viable or are less viable than what is observable in pests of the same species that are not contacted with the iRNA molecule.

Transgenic crops that produce substances that provide protection from insect feeding are vulnerable to adaptation by the target insect pest population reducing the durability of the benefits of the insect protection substance(s). Traditionally, delays in insect pest adaptation to transgenic crops are achieved by (1) the planting of “refuges” (crops that do not contain the pesticidal substances, and therefore allow survival of insects that are susceptible to the pesticidal substance(s)); and/or (2) combining insecticidal substances with multiple modes of action against the target pests, so that individuals that are resistant to one mode of action are killed by a second mode of action.

In some examples, iRNA molecules (e.g., expressed from a transgene in a host plant) represent new modes of action for combining with Bacillus thuringiensis insecticidal protein technology and/or lethal RNAi technology in Insect Resistance Management gene pyramids to mitigate against the development of insect populations resistant to either of these control technologies.

Parental RNAi may result in some embodiments in a type of pest control that is different from the control obtained by lethal RNAi, and which may be combined with lethal RNAi to result in synergistic pest control. Thus, in particular embodiments, iRNA molecules for the post-transcriptional inhibition of one or more target gene(s) in a coleopteran plant pest can be combined with other iRNA molecules to provide redundant RNAi targeting and synergistic RNAi effects.

Parental RNAi (pRNAi) that causes egg mortality or loss of egg viability has the potential to bring further durability benefits to transgenic crops that use RNAi and other mechanisms for insect protection. pRNAi prevents exposed insects from producing progeny, and therefore from passing on to the next generation any alleles they carry that confer resistance to the pesticidal substance(s). pRNAi is particularly useful in extending the durability of insect-protected transgenic crops when it is combined with one or more additional pesticidal substances that provide protection from the same pest populations. Such additional pesticidal substances may in some embodiments include, for example, dsRNA; larval-active dsRNA; adult-active dsRNA, insecticidal proteins (such as those derived from Bacillus thuringiensis or other organisms); and other insecticidal substances. This benefit arises because insects that are resistant to the pesticidal substances occur as a higher proportion of the population in the transgenic crop than in the refuge crop. If a ratio of resistance alleles to susceptible alleles that are passed on to the next generation is lower in the presence of pRNAi than in the absence of pRNAi, the evolution of resistance will be delayed.

For example, pRNAi may not reduce the number of individuals in a first pest generation that are inflicting damage on a plant expressing an iRNA molecule. However, the ability of such pests to sustain an infestation through subsequent generations may be reduced. Conversely, lethal RNAi may kill pests that already are infesting the plant. When pRNAi is combined with lethal RNAi, pests that are contacted with a parental iRNA molecule may breed with pests from outside the system that have not been contacted with the iRNA, however, the progeny of such a mating may be non-viable or less viable, and thus may be unable to infest the plant. At the same time, pests that are contacted with a lethal iRNA molecule may be directly affected. The combination of these two effects may be synergistic; i.e., the combined pRNAi and lethal RNAi effect may be greater than the sum of the pRNAi and lethal RNAi effects independently. pRNAi may be combined with lethal RNAi, for example, by providing a plant that expresses both lethal and parental iRNA molecules; by providing in the same location a first plant that expresses lethal iRNA molecules and a second plant that expresses parental iRNA molecules; and/or by contacting female and/or male pests with the pRNAi molecule, and subsequently releasing the contacted pests into the plant environment, such that they can mate unproductively with the plant pests.

Some embodiments provide methods for reducing the damage to a host plant (e.g., a corn plant) caused by a coleopteran pest that feeds on the plant, wherein the method comprises providing in the host plant a transformed plant cell expressing at least one nucleic acid molecule of the invention, wherein the nucleic acid molecule(s) functions upon being taken up by the pest(s) to inhibit the expression of a target polynucleotide within the pest(s), which inhibition of expression results in reduced reproduction, for example, in addition to mortality and/or reduced growth of the pest(s), thereby reducing the damage to the host plant caused by the pest. In some embodiments, the nucleic acid molecule(s) comprise dsRNA molecules. In these and further embodiments, the nucleic acid molecule(s) comprise dsRNA molecules that each comprise more than one polynucleotide that is specifically hybridizable to a nucleic acid molecule expressed in a coleopteran pest cell. In some embodiments, the nucleic acid molecule(s) consist of one polynucleotide that is specifically hybridizable to a nucleic acid molecule expressed in a coleopteran pest cell.

In other embodiments, a method for increasing the yield of a corn crop is provided, wherein the method comprises introducing into a corn plant at least one nucleic acid molecule of the invention; and cultivating the corn plant to allow the expression of an iRNA molecule comprising the nucleic acid, wherein expression of an iRNA molecule comprising the nucleic acid inhibits coleopteran pest damage and/or growth, thereby reducing or eliminating a loss of yield due to coleopteran pest infestation. In some embodiments, the iRNA molecule is a dsRNA molecule. In these and further embodiments, the nucleic acid molecule(s) comprise dsRNA molecules that each comprise more than one polynucleotide that is specifically hybridizable to a nucleic acid molecule expressed in a coleopteran pest cell. In some embodiments, the nucleic acid molecule(s) consists of one polynucleotide that is specifically hybridizable to a nucleic acid molecule expressed in a coleopteran pest cell.

In particular embodiments, a method for increasing the yield of a plant crop is provided, wherein the method comprises introducing into a female coleopteran pest (e.g., by injection, by ingestion, by spraying, and by expression from a DNA) at least one nucleic acid molecule of the invention; and releasing the female pest into the crop, wherein mating pairs including the female pest are unable or less able to produce viable offspring, thereby reducing or eliminating a loss of yield due to coleopteran pest infestation. In particular embodiments, such a method provides control of subsequent generations of the pest. In similar embodiments, the method comprises introducing the nucleic acid molecule of the invention into a male coleopteran pest, and releasing the male pest into the crop (e.g., wherein pRNAi male pests produce less sperm than untreated controls). For example, given that WCR females typically mate only once, these pRNAi female and/or males can be used in competition to overwhelm native WCR insects for mates. In some embodiments, the nucleic acid molecule is a DNA molecule that is expressed to produce an iRNA molecule. In some embodiments, the nucleic acid molecule is a dsRNA molecule. In these and further embodiments, the nucleic acid molecule(s) comprise dsRNA molecules that each comprise more than one polynucleotide that is specifically hybridizable to a nucleic acid molecule expressed in a coleopteran pest cell. In some embodiments, the nucleic acid molecule(s) consists of one polynucleotide that is specifically hybridizable to a nucleic acid molecule expressed in a coleopteran pest cell.

In alternative embodiments, a method for modulating the expression of a target gene in a coleopteran pest is provided, the method comprising: transforming a plant cell with a vector comprising a polynucleotide encoding at least one iRNA molecule of the invention, wherein the polynucleotide is operatively-linked to a promoter and a transcription termination element; culturing the transformed plant cell under conditions sufficient to allow for development of a plant cell culture including a plurality of transformed plant cells; selecting for transformed plant cells that have integrated the polynucleotide into their genomes; screening the transformed plant cells for expression of an iRNA molecule encoded by the integrated polynucleotide; selecting a transgenic plant cell that expresses the iRNA molecule; and feeding the selected transgenic plant cell to the coleopteran pest. Plants may also be regenerated from transformed plant cells that express an iRNA molecule encoded by the integrated nucleic acid molecule. In some embodiments, the iRNA molecule is a dsRNA molecule. In these and further embodiments, the nucleic acid molecule(s) comprise dsRNA molecules that each comprise more than one polynucleotide that is specifically hybridizable to a nucleic acid molecule expressed in a coleopteran pest cell. In some embodiments, the nucleic acid molecule(s) consists of one polynucleotide that is specifically hybridizable to a nucleic acid molecule expressed in a coleopteran pest cell.

iRNA molecules of the invention can be incorporated within the seeds of a plant species (e.g., corn), either as a product of expression from a recombinant gene incorporated into a genome of the plant cells, or as incorporated into a coating or seed treatment that is applied to the seed before planting. A plant cell comprising a recombinant gene is considered to be a transgenic event. Also included in embodiments of the invention are delivery systems for the delivery of iRNA molecules to coleopteran pests. For example, the iRNA molecules of the invention may be directly introduced into the cells of a pest(s). Methods for introduction may include direct mixing of iRNA into the diet of the coleopteran pest (e.g., by mixing with plant tissue from a host for the pest), as well as application of compositions comprising iRNA molecules of the invention to host plant tissue. For example, iRNA molecules may be sprayed onto a plant surface. Alternatively, an iRNA molecule may be expressed by a microorganism, and the microorganism may be applied onto the plant surface, or introduced into a root or stem by a physical means such as an injection. As discussed, supra, a transgenic plant may also be genetically engineered to express at least one iRNA molecule in an amount sufficient to kill the coleopteran pests or their progeny known to infest the plant. iRNA molecules produced by chemical or enzymatic synthesis may also be formulated in a manner consistent with common agricultural practices, and used as spray-on products for controlling plant damage by a coleopteran pest. The formulations may include the appropriate adjuvants (e.g., stickers and wetters) required for efficient foliar coverage, as well as UV protectants to protect iRNA molecules (e.g., dsRNA molecules) from UV damage. Such additives are commonly used in the bioinsecticide industry, and are well known to those skilled in the art. Such applications may be combined with other spray-on insecticide applications (biologically based or otherwise) to enhance plant protection from coleopteran pests.

All references, including publications, patents, and patent applications, cited herein are hereby incorporated by reference to the extent they are not inconsistent with the explicit details of this disclosure, and are so incorporated to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

The following Examples are provided to illustrate certain particular features and/or aspects. These Examples should not be construed to limit the disclosure to the particular features or aspects described.

### EXAMPLES

### Example 1

### Materials and Methods

Sample preparation and bioassays for Diabrotica larval feeding assays.

A number of dsRNA molecules (including those corresponding to kruppel) were synthesized and purified using a MEGAscript® RNAi kit (LIFE TECHNOLOGIES) or HiScribe®T7 In Vitro Transcription kit (NEW ENGLAND BIOLABS). The purified dsRNA molecules were prepared in TE buffer, and all bioassays contained a control treatment consisting of this buffer, which served as a background check for mortality or growth inhibition of WCR. The concentrations of dsRNA molecules in the bioassay buffer were measured using a NANODROP™ 8000 spectrophotometer (THERMO SCIENTIFIC, Wilmington, Del.).

Samples were tested for insect activity in bioassays conducted with neonate insect larvae on artificial insect diet. WCR eggs were obtained from CROP CHARACTERISTICS, INC. (Farmington, Minn.).

The bioassays were conducted in 128-well plastic trays specifically designed for insect bioassays (C-D INTERNATIONAL, Pitman, N.J.). Each well contained approximately 1.0 mL of a diet designed for growth of coleopteran insects. A 60 μL aliquot of dsRNA sample was delivered by pipette onto the 1.5 cm² diet surface of each well (40 μL/cm²). dsRNA sample concentrations were calculated as the amount of dsRNA per square centimeter (ng/cm²) of surface area in the well. The treated trays were held in a fume hood until the liquid on the diet surface evaporated or was absorbed into the diet.

Within a few hours of eclosion, individual larvae were picked up with a moistened camel hair brush and deposited on the treated diet (one or two larvae per well). The infested wells of the 128-well plastic trays were then sealed with adhesive sheets of clear plastic, and vented to allow gas exchange. Bioassay trays were held under controlled environmental conditions (28° C., ˜40% Relative Humidity, 16:8 (Light:Dark)) for 9 days, after which time the total number of insects exposed to each sample, the number of dead insects, and the weight of surviving insects were recorded. Percent mortality, average live weights, and growth inhibition were calculated for each treatment. Stunting was defined as a decrease in average live weights. Growth inhibition (GI) was calculated as follows:

GI=[1−(TWIT/TNIT)/(TWIBC/TNIBC)],

where TWIT is the Total Weight of live Insects in the Treatment;

### TNIT is the Total Number of Insects in the Treatment;

TWIBC is the Total Weight of live Insects in the Background Check (Buffer control); and
TNIBC is the Total Number of Insects in the Background Check (Buffer control).

The GI₅₀ is determined to be the concentration of sample in the diet at which the GI value is 50%. The LC₅₀ (50% Lethal Concentration) is recorded as the concentration of sample in the diet at which 50% of test insects are killed. Statistical analysis was done using JMP™ software (SAS, Cary, N.C.).

### Example 2

### Identification of Candidate Target Genes from Diabrotica

Insects from multiple stages of WCR (Diabrotica virgifera virgifera LeConte) development were selected for pooled transcriptome analysis to provide candidate target gene sequences for control by RNAi transgenic plant insect protection technology.

In one exemplification, total RNA was isolated from about 0.9 gm whole first-instar WCR larvae; (4 to 5 days post-hatch; held at 16° C.), and purified using the following phenol/TRI REAGENT™-based method (MOLECULAR RESEARCH CENTER, Cincinnati, Ohio).

Larvae were homogenized at room temperature in a 15 mL homogenizer with 10 mL of TRI REAGENT® until a homogenous suspension was obtained. Following 5 min. incubation at room temperature, the homogenate was dispensed into 1.5 mL microfuge tubes (1 mL per tube), 200 μL of chloroform was added, and the mixture was vigorously shaken for 15 seconds. After allowing the extraction to sit at room temperature for 10 min, the phases were separated by centrifugation at 12,000×g at 4° C. The upper phase (comprising about 0.6 mL) was carefully transferred into another sterile 1.5 mL tube, and an equal volume of room temperature isopropanol was added. After incubation at room temperature for 5 to 10 min, the mixture was centrifuged 8 min at 12,000×g (4° C. or 25° C.).

The supernatant was carefully removed and discarded, and the RNA pellet was washed twice by vortexing with 75% ethanol, with recovery by centrifugation for 5 min at 7,500×g (4° C. or 25° C.) after each wash. The ethanol was carefully removed, the pellet was allowed to air-dry for 3 to 5 min, and then was dissolved in nuclease-free sterile water. RNA concentration was determined by measuring the absorbance (A) at 260 nm and 280 nm. A typical extraction from about 0.9 gm of larvae yielded over 1 mg of total RNA, with an A₂₆₀/A₂₈₀ ratio of 1.9. The RNA thus extracted was stored at −80° C. until further processed.

RNA quality was determined by running an aliquot through a 1% agarose gel. The agarose gel solution was made using autoclaved 10×TAE buffer (Tris-acetate EDTA; 1× concentration is 0.04 M Tris-acetate, 1 mM EDTA (ethylenediamine tetra-acetic acid sodium salt), pH 8.0) diluted with DEPC (diethyl pyrocarbonate)-treated water in an autoclaved container. 1×TAE was used as the running buffer. Before use, the electrophoresis tank and the well-forming comb were cleaned with RNAseAway™ (INVITROGEN INC., Carlsbad, Calif.). Two μL of RNA sample were mixed with 8 μL of TE buffer (10 mM Tris HCl pH 7.0; 1 mM EDTA) and 10 μL of RNA sample buffer (NOVAGEN® Catalog No 70606; EMD4 Bioscience, Gibbstown, N.J.). The sample was heated at 70° C. for 3 min, cooled to room temperature, and 5 μL (containing 1 μg to 2 μg RNA) were loaded per well. Commercially available RNA molecular weight markers were simultaneously run in separate wells for molecular size comparison. The gel was run at 60 volts for 2 hr.

A normalized cDNA library was prepared from the larval total RNA by a commercial service provider (EUROFINS MWG Operon, Huntsville, Ala.), using random priming. The normalized larval cDNA library was sequenced at ½ plate scale by GS FLX 454 Titanium™ series chemistry at EUROFINS MWG Operon, which resulted in over 600,000 reads with an average read length of 348 bp. 350,000 reads were assembled into over 50,000 contigs. Both the unassembled reads and the contigs were converted into BLASTable databases using the publicly available program, FORMATDB (available from NCBI).

Total RNA and normalized cDNA libraries were similarly prepared from materials harvested at other WCR developmental stages. A pooled transcriptome library for target gene screening was constructed by combining cDNA library members representing the various developmental stages.

Candidate genes for RNAi targeting were selected using information regarding lethal effects of particular genes in other insects such as Drosophila and Tribolium. Kruppel is a gap gene, a mutation or deletion of which produces a gap in the body segmentation. For example, the gap gene kruppel, a transcription factor necessary for the establishment of anterior-posterior polarity during early embryonic development, was selected based on overall conservation of kruppel function in Drosophila and Tribolium (Cerny (2005) Development 132:5353-63; Treisman (1989) Nature 341:335-7; Nibu (2003) Mol. Cell Biol. 23:3990-9).

TBLASTN searches using candidate protein coding sequences were run against BLASTable databases containing the unassembled Diabrotica sequence reads or the assembled contigs. Significant hits to a Diabrotica sequence (defined as better than e⁻²⁰ for contigs homologies and better than e⁻¹⁰ for unassembled sequence reads homologies) were confirmed using BLASTX against the NCBI non-redundant database. The results of this BLASTX search confirmed that the Diabrotica homolog candidate gene sequences identified in the TBLASTN search indeed comprised Diabrotica genes, or were the best hit available in the Diabrotica sequences to the non-Diabrotica candidate gene sequence. In most cases, Tribolium candidate genes which were annotated as encoding a protein gave an unambiguous sequence homology to a sequence or sequences in the Diabrotica transcriptome sequences. In a few cases, it was clear that some of the Diabrotica contigs or unassembled sequence reads selected by homology to a non-Diabrotica candidate gene overlapped, and that the assembly of the contigs had failed to join these overlaps. In those cases, SEQUENCHER™ v4.9 (GENE CODES CORPORATION, Ann Arbor, Mich.) was used to assemble the sequences into longer contigs.

Additional transcriptome sequencing of D. v. virgifera has been previously described. Euyn et al. (2014) PLoS One 9(4):e94052. In another exemplification, using Illumina™ paired-end as well as 454 Titanium sequencing technologies, a total of ˜700 gigabases were sequenced from cDNA prepared from eggs (15,162,017 Illumina™ paired-end reads after filtering), neonates (721,697,288 Illumina™ paired-end reads after filtering), and midguts of third instar larvae (44,852,488 Illumina™ paired-end reads and 415,742 Roche 454 reads, both after filtering). De novo transcriptome assembly was performed using Trinity (Grabherr et al. (2011) Nat. Biotechnol. 29(7):644-52) for each of three samples as well as for the pooled dataset. The pooled assembly resulted in 163,871 contigs with an average length of 914 bp. The amino acid sequences of KRUPPEL from Drosophila or Tribolium were used as query sequences to search the rootworm transcriptome and genome database (unpublished) with tBLASTN using a cut-off E value of 10⁻⁵. The deduced amino acid sequences were aligned with ClustalX™ and edited with GeneDoc™ software.

A candidate target gene was identified that may lead to coleopteran pest mortality or inhibition of growth, development, or reproduction in WCR, including transcripts: SEQ ID NO:1 and SEQ ID NO:2, with subsequence sub-region SEQ ID NO:4. These sequences encode a KRUPPEL protein or sub-regions thereof, which are involved in adult morphogenesis in western corn rootworms. Transcription factor E93 triggers adult morphogenesis in Tribolium castaneum and other insects (Belles and Santos (2014) Insect Biochem. Mol. Biol. 52: 60-8).

The polynucleotide of SEQ ID NO:1 and SEQ ID NO:2 are novel. The sequences are not provided in public databases and are not disclosed in WO/2011/025860; U.S. Patent Application No. 20070124836; U.S. Patent Application No. 20090306189; U.S. Patent Application No. US20070050860; U.S. Patent Application No. 20100192265; or U.S. Pat. No. 7,612,194. There was no significant homologous nucleotide sequence found with a search in GENBANK. The closest homolog of the Diabrotica KRUPPEL amino acid sequence (SEQ ID NO:3) is a Tribolium castaneum protein having GENBANK Accession No. NP_001034527.2 (71% similar; 60% identical over the homology region).

Full-length or partial clones of sequences of Diabrotica candidate kruppel gene were used to generate PCR amplicons for dsRNA synthesis. dsRNA was also amplified from a DNA clone comprising the coding region for a yellow fluorescent protein (YFP) (SEQ ID NO:11; Shagin et al. (2004) Mol. Biol. Evol. 21:841-850).

### Example 3

### Amplification of Target Genes from Diabrotica

Primers were designed to amplify portions of coding regions of each target gene by PCR. See Table 1. Where appropriate, a T7 phage promoter sequence (TAATACGACTCACTATAGGG (SEQ ID NO:5)) was incorporated into the 5′ ends of the amplified sense or antisense strands. See Table 1. Total RNA was extracted from WCR, and first-strand cDNA was used as template for PCR reactions using opposing primers positioned to amplify all or part of the native target gene sequence.

TABLE 1
Primers and Primer Pairs used to amplify portions of coding regions of
exemplary kruppel and YFP target genes.

| | Gene (Region) | Primer_ID | Sequence |
|---|---|---|---|
| Pair 1 | kruppel Reg1 | kruppel_T7F | TAATACGACTCACTATAGGGCCTGTGGAGTATGTAATAG |
| | | | (SEQ ID NO: 6) |
| | | kruppel_T7R | TAATACGACTCACTATAGGGGATTGGAGTCTGAGAATT |
| | | | (SEQ ID NO: 7) |
| Pair 2 | YFP | YFP-F_T7 | TTAATACGACTCACTATAGGGAGACACCATGGGCTCCAG |
| | | | CGGCGCCC ( SEQ ID NO: 42) |
| | | YFP-R_T7 | TTAATACGACTCACTATAGGGAGAAGATCTTGAAGGCGC |
| | | | TCTTCAGG (SEQ ID NO: 45) |
| Pair 3 | GFP | GFP-F_T7 | TAATACGACTCACTATAGGGGGTGATGCTACATACGGAA |
| | | | AG (SEQ ID NO: 8) |
| | | GFP-R_T7 | TAATACGACTCACTATAGGGTTGTTTGTCTGCCGTGAT |
| | | | (SEQ ID NO: 9) |

### Example 4

### RNAi Constructs

Template preparation by PCR and dsRNA synthesis

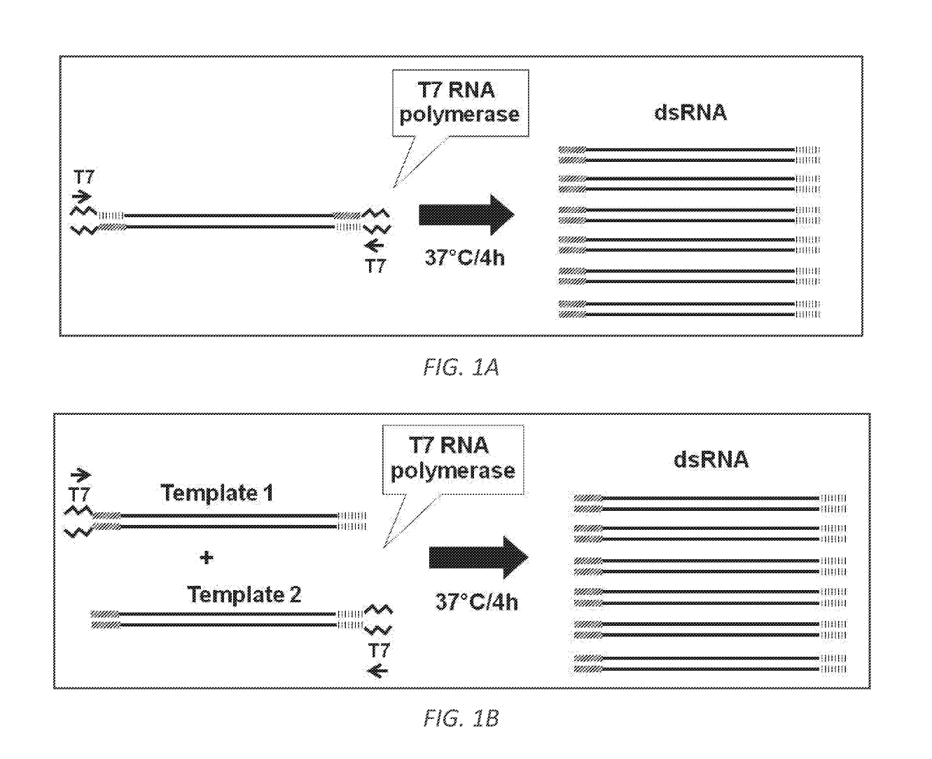

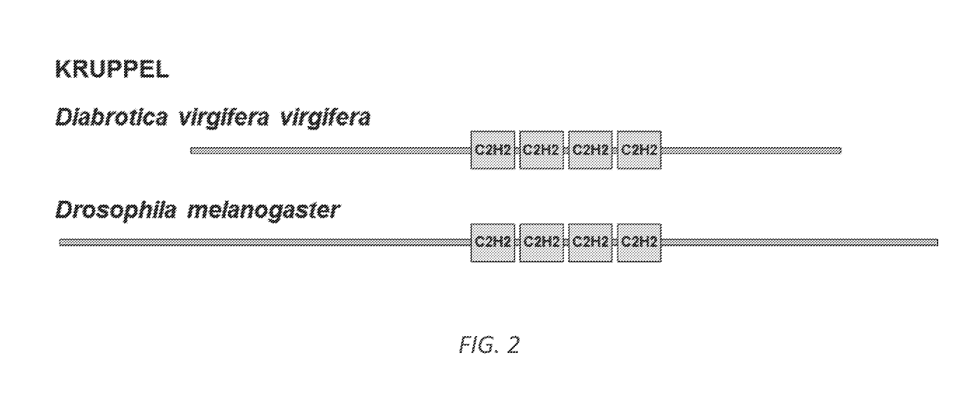

The strategies used to provide specific templates for kruppel dsRNA production are shown in FIG. 1A and FIG. 1B. Template DNA intended for use in kruppel Reg1 dsRNA synthesis was prepared by PCR using Primer Pair 1 (Table 1) and (as PCR template) first-strand cDNA prepared from total RNA. For the kruppel Reg1 selected target gene region, two separate PCR amplifications were performed. The first PCR amplification introduced a T7 promoter sequence at the 5′ end of the amplified sense strands. The second reaction incorporated the T7 promoter sequence at the 5′ ends of the antisense strands. The two PCR amplified fragments for each region of the target genes were then mixed in approximately equal amounts, and the mixture was used as transcription template for dsRNA production. The sequence of kruppel Reg1 dsRNA template amplified with the particular primers is disclosed as SEQ ID NO:4.

For the YFP negative control, a single PCR amplification was performed. FIG. 1B. The PCR amplification introduced a T7 promoter sequence at the 5′ ends of the amplified sense and antisense strands. The two PCR amplified fragments for each region of the target genes were then mixed in approximately equal amounts, and the mixture was used as transcription template for dsRNA production. FIG. 1B. dsRNA for the negative control YFP coding region (SEQ ID NO:11) was produced using Primer Pair 2 (Table 1) and a DNA clone of the YFP coding region as template. A GFP negative control was amplified from the pIZT/V5-His expression vector (Invitrogen) using Primer Pair 3 (Table 1). The PCR product amplified for kruppel and GFP were used as a template for in vitro synthesis of dsRNAs using the MEGAscript high-yield transcription kit (Applied Biosystems Inc., Foster City, Calif.). The synthesized dsRNAs were purified using the RNeasy Mini kit (Qiagen, Valencia, Calif.) or an AMBION® MEGAscript® RNAi kit essentially as prescribed by the manufacturer's instructions. dsRNA preparations were quantified using a NANODROP™ 8000 spectrophotometer (THERMO SCIENTIFIC, Wilmington, Del.) or equivalent means and analyzed by gel electrophoresis to determine purity.

### Example 5

### Screening of Candidate Target Genes in Diabrotica Larvae

Replicated bioassays demonstrated that ingestion of synthetic dsRNA preparations derived from the kruppel Reg1 target gene sequence identified in EXAMPLE 2 caused mortality and growth inhibition of western corn rootworm larvae when administered to WCR in diet-based assays. Table 2.

TABLE 2
Results of diet-based feeding bioassays of WCR larvae following 9-
day exposure to a single dose of dsRNAs. ANOVA analysis found some
significance differences in Mean % Mortality (Mort.) and Mean % Growth
Inhibition (GI). Means were separated using the Tukey-Kramer test.

| | Dose | No. Rows | *Mean % Mortality +/− | *Mean GI +/− |
|---|---|---|---|---|
| Sample Name | (ng/cm²) | (Replications) | SEM | SEM |
| kruppel Reg 1 | 500 | 6 | 66.67 ± 15.54 (A) | 0.72 ± 0.16 (A) |
| TE buffer** | 0 | 6 | 12.75 ± 6.15 (B) | 0.06 ± 0.06 (B) |
| Water | 0 | 5 | 12.94 ± 4.71 (B) | 0.08 ± 0.05 (B) |
| YFP dsRNA*** | 500 | 6 | 9.93 ± 4.45 (B) | 0.16 ± 0.07 (B) |

*SEM = Standard Error of the Mean. Letters in parentheses designate statistical levels. Levels not connected by same letter are significantly different (p < 0.05).
**TE = Tris HCl (1 mM) plus EDTA (1 mM) buffer, pH 7.2.
***YFP = Yellow Fluorescent Protein

It has previously been suggested that certain genes of Diabrotica spp. may be exploited for RNAi-mediated insect control. See U.S. Patent Publication No. 2007/0124836, which discloses 906 sequences, and U.S. Pat. No. 7,614,924, which discloses 9,112 sequences. However, it was determined that many genes suggested to have utility for RNAi-mediated insect control are not efficacious in controlling Diabrotica. It was also determined that kruppel Reg1 provided surprising and unexpected control of Diabrotica, compared to other genes suggested to have utility for RNAi-mediated insect control.

For example, Annexin, Beta Spectrin 2, and mtRP-L4 were each suggested in U.S. Pat. No. 7,614,924 to be efficacious in RNAi-mediated insect control. SEQ ID NO:12 is the DNA sequence of Annexin Region 1 and SEQ ID NO:13 is the DNA sequence of Annexin Region 2. SEQ ID NO:14 is the DNA sequence of Beta Spectrin 2 Region 1 and SEQ ID NO:15 is the DNA sequence of Beta Spectrin 2 Region 2. SEQ ID NO:16 is the DNA sequence of mtRP-L4 Region 1 and SEQ ID NO:17 is the DNA sequence of mtRP-L4 Region 2.

Each of the aforementioned sequences was used to produce dsRNA by the dual Primer Pair methods of EXAMPLE 4, and the dsRNAs were each tested by the diet-based bioassay methods described above. A YFP sequence (SEQ ID NO:11) was also used to produce dsRNA as a negative control. Table 3 lists the sequences of the primers used to produce the Annexin, Beta Spectrin 2, mtRP-L4, and YFP dsRNA molecules. Table 4 presents the results of diet-based feeding bioassays of WCR larvae following 9-day exposure to these dsRNA molecules. Replicated bioassays demonstrated that ingestion of these dsRNAs resulted in no mortality or growth inhibition of western corn rootworm larvae above that seen with control samples of TE buffer, YFP dsRNA, or water.

TABLE 3
Primers and Primer Pairs used to amplify portions of coding regions of
genes.

| | Gene Region | Primer ID | Sequence |
|---|---|---|---|
| Pair 4 | Annexin | Ann-F1_T7 | TTAATACGACTCACTATAGGGAGAGCTCCAACAGTGG |
| | Region 1 | | TTCCTTATC (SEQ ID NO: 18) |
| | Annexin | Ann-R1 | CTAATAATTCTTTTTTAATGTTCCTGAGG |
| | Region 1 | | (SEQ ID NO: 19) |
| Pair 5 | Annexin | Ann-F1 | GCTCCAACAGTGGTTCCTTATC (SEQ ID NO: 20) |
| | Region 1 | | |
| | Annexin | Ann-R1_T7 | TTAATACGACTCACTATAGGGAGACTAATAATTCTTT |
| | Region 1 | | TTTAATGTTCCTGAGG (SEQ ID NO: 21) |
| Pair 6 | Annexin | Ann-F2_T7 | TTAATACGACTCACTATAGGGAGATTGTTACAAGCTG |
| | Region 2 | | GAGAACTTCTC (SEQ ID NO: 22) |
| | Annexin | Ann-R2 | CTTAACCAACAACGGCTAATAAGG |
| | Region 2 | | (SEQ ID NO: 23) |
| Pair 7 | Annexin | Ann-F2 | TTGTTACAAGCTGGAGAACTTCTC |
| | Region 2 | | (SEQ ID NO: 24) |
| | Annexin | Ann-R2T7 | TTAATACGACTCACTATAGGGAGACTTAACCAACAAC |
| | Region 2 | | GGCTAATAAGG (SEQ ID NO: 25) |
| Pair 8 | Beta-Spect2 | Betasp2- | TTAATACGACTCACTATAGGGAGAAGATGTTGGCTGC |
| | Region 1 | F1_T7 | ATCTAGAGAA (SEQ ID NO: 26) |
| | Beta-Spect2 | Betasp2-R1 | GTCCATTCGTCCATCCACTGCA |
| | Region 1 | | (SEQ ID NO: 27) |
| Pair 9 | Beta-Spect2 | Betasp2-F1 | AGATGTTGGCTGCATCTAGAGAA |
| | Region 1 | | (SEQ ID NO: 28) |
| | Beta-Spect2 | Betasp2- | TTAATACGACTCACTATAGGGAGAGTCCATTCGTCCA |
| | Region 1 | R1_T7 | TCCACTGCA (SEQ ID NO: 29) |
| Pair 10 | Beta-Spect2 | Betasp2- | TTAATACGACTCACTATAGGGAGAGCAGATGAACACC |
| | Region 2 | F2_T7 | AGCGAGAAA (SEQ ID NO: 30) |
| | Beta-Spect2 | Betasp2-R2 | CTGGGCAGCTTCTTGTTTCCTC (SEQ ID NO: 31) |
| | Region 2 | | |
| Pair 11 | Beta-Spect2 | Betasp2-F2 | GCAGATGAACACCAGCGAGAAA (SEQ ID NO: 32) |
| | Region 2 | | |
| | Beta-Spect2 | Betasp2- | TTAATACGACTCACTATAGGGAGACTGGGCAGCTTCT |
| | Region 2 | R2_T7 | TGTTTCCTC (SEQ ID NO: 33) |
| Pair 12 | mtRP-L4 | L4-F1_T7 | TTAATACGACTCACTATAGGGAGAAGTGAAATGTTAG |
| | Region 1 | | CAAATATAACATCC (SEQ ID NO: 34) |
| | mtRP-L4 | L4-R1 | ACCTCTCACTTCAAATCTTGACTTTG |
| | Region 1 | | (SEQ ID NO: 35) |
| Pair 13 | mtRP-L4 | L4-F1 | AGTGAAATGTTAGCAAATATAACATCC |
| | Region 1 | | (SEQ ID NO: 36) |
| | mtRP-L4 | L4-R1_T7 | TTAATACGACTCACTATAGGGAGAACCTCTCACTTCA |
| | Region 1 | | AATCTTGACTTTG (SEQ ID NO: 37) |
| Pair 14 | mtRP-L4 | L4-F2_T7 | TTAATACGACTCACTATAGGGAGACAAAGTCAAGATT |
| | Region 2 | | TGAAGTGAGAGGT (SEQ ID NO: 38) |
| | mtRP-L4 | L4-R2 | CTACAAATAAAACAAGAAGGACCCC |
| | Region 2 | | (SEQ ID NO: 39) |
| Pair 15 | mtRP-L4 | L4-F2 | CAAAGTCAAGATTTGAAGTGAGAGGT |
| | Region 2 | | (SEQ ID NO: 40) |
| | mtRP-L4 | L4-R2_T7 | TTAATACGACTCACTATAGGGAGACTACAAATAAAAC |
| | Region 2 | | AAGAAGGACCCC (SEQ ID NO: 41) |
| Pair 16 | YFP | YFP-F_T7 | TTAATACGACTCACTATAGGGAGACACCATGGGCTCC |
| | | | AGCGGCGCCC (SEQ ID NO: 42) |
| | YFP | YFP-R | AGATCTTGAAGGCGCTCTTCAGG |
| | | | (SEQ ID NO: 43) |
| Pair 17 | YFP | YFP-F | CACCATGGGCTCCAGCGGCGCCC |
| | | | (SEQ ID NO: 44) |
| | YFP | YFP-R_T7 | TTAATACGACTCACTATAGGGAGAAGATCTTGAAGGC |
| | | | GCTCTTCAGG ( SEQ ID NO: 45) |

TABLE 4
Results of diet feeding assays obtained
with western corn rootworm larvae.

| | | Mean weight | | |
|---|---|---|---|---|
| | Dose | per insect | Mean % | Mean Growth |
| Gene Name | (ng/cm²) | (mg) | Mortality | Inhibition |
| annexin-region 1 | 1000 | 0.545 | 0 | −0.262 |
| annexin-region 2 | 1000 | 0.565 | 0 | −0.301 |
| beta spectrin2 | 1000 | 0.340 | 12 | −0.014 |
| region 1 | | | | |
| beta spectrin2 | 1000 | 0.465 | 18 | −0.367 |
| region 2 | | | | |
| mtRP-L4 region 1 | 1000 | 0.305 | 4 | −0.168 |
| mtRP-L4 region 2 | 1000 | 0.305 | 7 | −0.180 |
| TE buffer | 0 | 0.430 | 13 | 0.000 |
| Water | 0 | 0.535 | 12 | 0.000 |
| YFP | 1000 | 0.480 | 9 | −0.386 |

### Example 6

### Sample Preparation and Bioassays for Diabrotica Adult Feeding Assays

Parental RNA interference (RNAi) in western corn rootworms was conducted by feeding dsRNA corresponding to the segments of kruppel target gene sequence to gravid adult females. Adult rootworms (<48 hrs. after emergence) were obtained from CROP CHARACTERISTICS, Inc. (Farmington, Minn.). Adults were reared at 23±1° C., relative humidity of >75%, and Light:Dark periods of 8 hr:16 hr for all bioassays. The insect rearing diet was adapted from Branson and Jackson (1988), J. Kansas Entomol. Soc. 61:353-55. Dry ingredients were added (48 gm/100 mL) to a solution comprising double distilled water with 2.9% agar and 5.6 mL of glycerol. In addition, 0.5 mL of a mixture comprising 47% propionic acid and 6% phosphoric acid solutions was added per 100 mL diet to inhibit microbial growth. The agar was dissolved in boiling water and the dry ingredients, glycerol, and propionic acid/phosphoric acid solution were added, mixed thoroughly, and poured to a depth of approximately 2 mm. Solidified diet plugs (about 4 mm in diameter by 2 mm height; 25.12 mm³) were cut from the diet with a No. 1 cork borer. Six adult males and females (24 to 48 hrs old) were maintained on untreated artificial diet and were allowed to mate for 4 days in 16 well trays (5.1 cm long×3.8 cm wide×2.9 high) with vented lids.

On day five, males were removed from the container, and females were fed on artificial diet surface plugs treated with 3 μl kruppel Reg1 (SEQ ID NO:4) gene-specific dsRNA (2 μg/diet plug; about 79.6 ng/mm³). Control treatments consisted of gravid females exposed to diet treated with the same concentration of GFP dsRNA (SEQ ID NO:10) or the same volume of water. GFP dsRNA was produced as described above using opposing primers having a T7 promoter sequence at their 5′ ends (SEQ ID NOs:8 and 9). Fresh artificial diet treated with dsRNA was provided every other day throughout the experiment. On day 11, females were transferred to oviposition cages (7.5 cm×5.5 cm×5.5 cm) (ShowMan box, Althor Products, Wilton, Conn.) containing autoclaved silty clay loam soil sifted through a 60-mesh sieve (Jackson (1986) Rearing and handling of Diabrotica virgifera and Diabrotica undecimpunctata howardi. Pages 25 to 47 in J. L. Krysan and T. A. Miller, eds. Methods for the study of pest Diabrotica. Springer-Verlag, New York). Females were allowed to oviposit for four days and the eggs were incubated in soil within the oviposition boxes for 10 days at 27° C. and then removed from the soil by washing the oviposition soil through a 60-mesh sieve. Eggs were treated with a solution of formaldehyde (500 μL formaldehyde in 5 mL double distilled water) and methyl-(butycarbamoy)-2-benzimidazole carbamate (0.025 g in 50 mL double distilled water) to prevent fungal growth. Females removed from the oviposition boxes and subsamples of eggs from each treatment were flash frozen in liquid nitrogen for subsequent expression analyses by quantitative qPCR (See EXAMPLE 7). The dishes were photographed with Dino-Lite Pro digital microscope (Torrance, Calif.) and total eggs counted using the cell counter function of Image J software (Schneider et al. (2012) Nat. Methods 9:671-5). Harvested eggs were held in Petri dishes on moistened filter paper at 28° C. and monitored for 15 days to determine egg viability. Six replications, each comprising three to six females, were run on separate days. The number of larvae hatching from each treatment was recorded daily until no further hatching was observed.

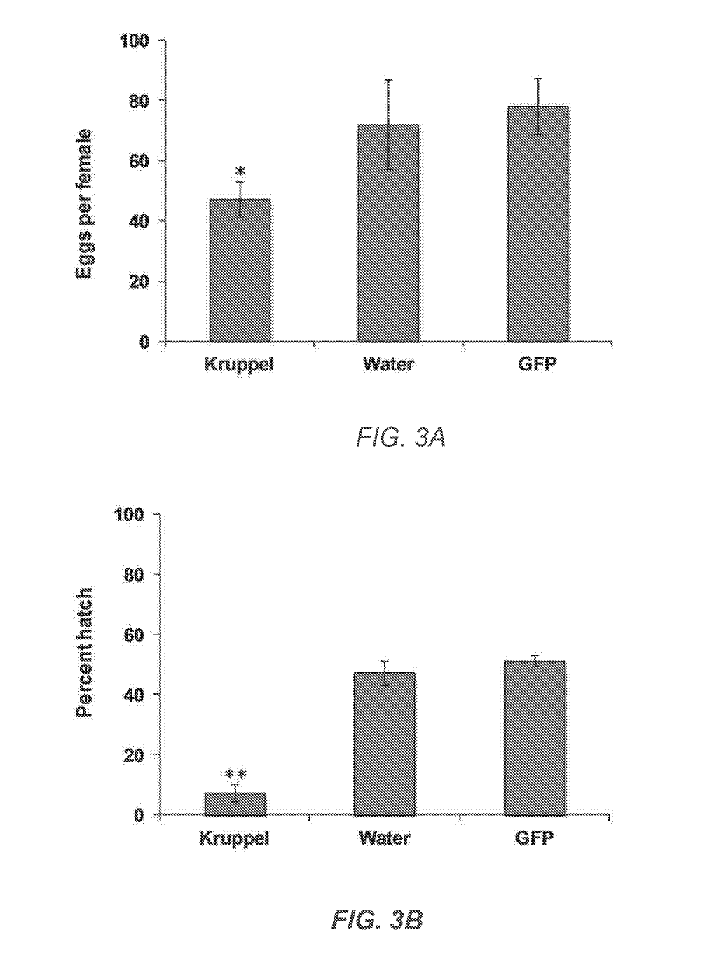

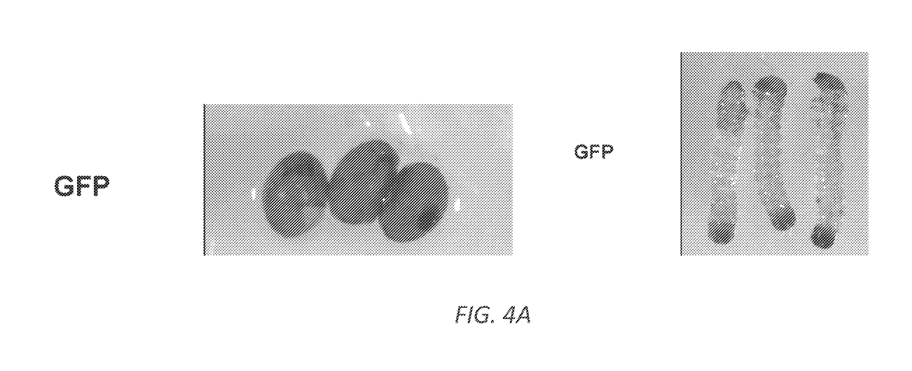

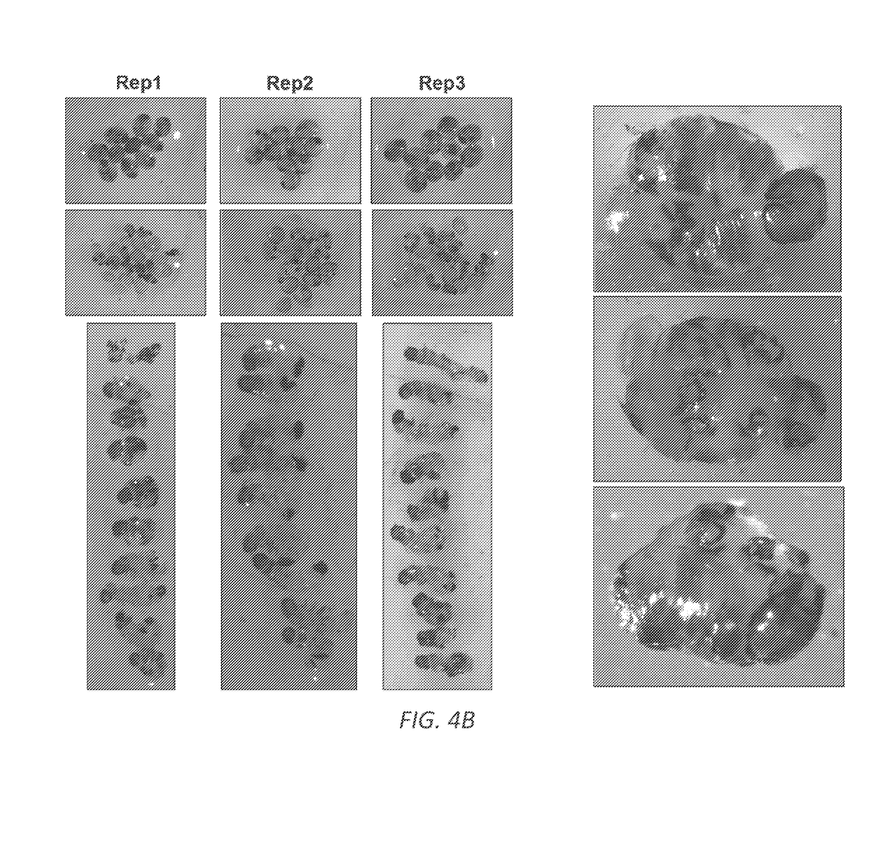

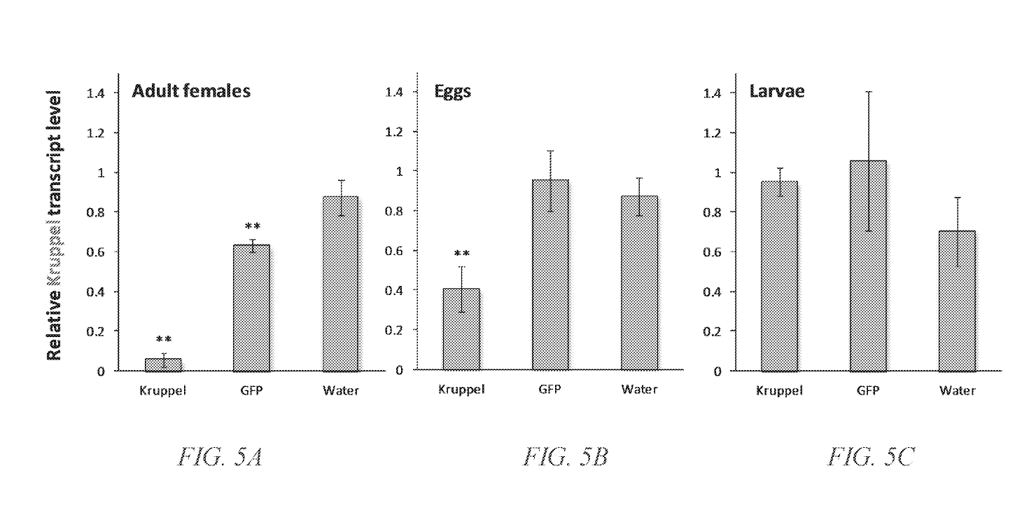

Ingestion of kruppel Reg1 dsRNA molecules by adult WCR females was demonstrated to have a surprising, dramatic and reproducible effect on egg viability. The mated females exposed to kruppel dsRNA produced approximately equal number of eggs to females exposed to untreated diet or diet treated with GFP dsRNA (FIG. 3A; Table 5). However, eggs collected from females that were exposed to kruppel dsRNA were not viable (FIG. 3B; Table 5). Adult females exposed to kruppel dsRNA had <3% of the eggs hatch.

FIGS. 3A and 3B graphically summarize the data of Table 5 regarding the effects that dsRNA treatments have on egg production and egg viability.

TABLE 5
Effect of kruppel dsRNA on WCR egg production and egg
viability after 11 days of ingestion on treated artificial diet.
Means were separated using Dunnett's test.

| | Egg numbers per female beetle | | | | | |
|---|---|---|---|---|---|---|
| | kruppel | | | Percent egg hatch | | |
| | Reg1 | GFP | | kruppel Reg1 | GFP | |
| | dsRNA | dsRNA | Water | dsRNA | dsRNA | Water |
| Average | 47.47* | 78.11 | 72.03 | 7.35** | 51.12 | 47.28 |
| SEM† | 5.81 | 9.13 | 15.01 | 2.78 | 1.87 | 4.02 |

†SEM—Standard Error of the Mean.
*indicates significance at p ≦ 0.10.
**indicates significance at p ≦ 0.05.

Embryos from unhatched eggs were dissected from each treatment to examine embryonic development and to estimate phenotypic responses to the parental RNAi (pRNAi) effect. The eggs deposited by WCR females treated with GFP dsRNA showed normal development. FIG. 4A. In contrast, eggs deposited by females treated with kruppel Reg1 dsRNA showed some embryonic development within the egg and, when dissected, were visibly shortened and appeared to be missing a number of abdominal and thoracic segments, although the response was variable among individual larvae. FIG. 4B. It is thus an unexpected finding of this invention that ingestion of kruppel dsRNA has a lethal or developmental effect on WCR eggs and larvae. It is further surprising and unexpected that kruppel dsRNA ingestion by adult WCR females dramatically impacts egg viability, while having no discernible dramatic effect on the adult females themselves.

The foregoing results clearly document the systemic nature of RNAi in western corn rootworms, and the potential to achieve a parental effect where genes associated with embryonic development are knocked down in the eggs of females that are exposed to dsRNA. Importantly, this is the first report of a pRNAi response to ingested dsRNA in western corn rootworms. A systemic response is indicated based on the observation of knockdown in tissues other than the alimentary canal where exposure and uptake of dsRNA is occurring. Because insects in general, and rootworms specifically, lack the RNA-dependent RNA polymerase that has been associated with systemic response in plants and nematodes, our results confirm that the dsRNA can be taken up by gut tissue and translocated to other tissues (e.g., developing ovarioles).

The ability to knock down the expression of genes involved in embryonic development such that the eggs do not hatch, offers a unique opportunity to achieve and improve control of western corn rootworms. Because adults readily feed on above-ground reproductive tissues (such as silks and tassels), adult rootworms can be exposed to iRNA control agents by transgenic expression of dsRNA to achieve root protection in the subsequent generation by preventing eggs from hatching. Delivery of the dsRNA through transgenic expression of dsRNA in corn plants, or by contact with surface-applied iRNAs, provides an important stacking partner for other transgenic approaches that target larvae directly and enhance the overall durability of pest management strategies.

### Example 7

### Real-Time PCR Analysis

Total RNA was isolated from the whole bodies of adult females, larvae hatched from treated females, and eggs using RNeasy mini Kit (Qiagen, Valencia, Calif.) following the manufacturer's recommendations. Before the initiation of the transcription reaction, the total RNA was treated with DNase to remove any gDNA using Quantitech reverse transcription kit (Qiagen, Valencia, Calif.). Total RNA (500 ng) was used to synthesize first strand cDNA as a template for real-time quantitative PCR (qPCR). The RNA was quantified spectrophotometrically at 260 nm and purity evaluated by agarose gel electrophoresis. Primers used for qPCR analysis were designed using Beacon designer software (Premier Biosoft International, Palo Alto, Calif.). The efficiencies of primer pairs were evaluated using 5 fold serial dilutions (1:1/5:1/25:1/125:1/625) in triplicate. Amplification efficiencies were higher than 96.1% for all the qPCR primer pairs used in this study. All primer combinations used in this study showed a linear correlation between the amount of cDNA template and the amount of PCR product. All correlation coefficients were larger than 0.99. The 7500 Fast System SDS v2.0.6 Software (Applied Biosystems) was used to determine the slope, correlation coefficients, and efficiencies. Three biological replications, each with two technical replications were used for qPCR analysis. qPCR was performed using SYBR green kit (Applied Biosystems Inc., Foster City, Calif.) and 7500 Fast System real-time PCR detection system (Applied Biosystems Inc., Foster City, Calif.). qPCR cycling parameters included 40 cycles each consisting of 95° C. for 3 sec, 58° C. for 30 sec, as described in the manufacturer's protocol (Applied Biosystems Inc., Foster City, Calif.). At the end of each PCR reaction, a melt curve was generated to confirm a single peak and rule out the possibility of primer-dimer and non-specific product formation. Relative quantification of the transcripts were calculated using the comparative 2^{-ΔΔCT} method and were normalized to β-actin.

FIGS. 5(A-C) graphically summarize the data of Table 6 showing the relative transcript levels of kruppel and GFP in eggs, adult females, and larvae compared to water controls. There is a surprising reduction in transcript levels in female adults and eggs. There is no reduction in transcript in larvae that hatched from treated females.

TABLE 6
Relative expression of kruppel in eggs, adult females, and larvae
exposed to dsRNA in treated artificial diet relative to GFP and
water controls. Means were separated using Dunnett's test.

| | | |
|---|---|---|
| Treatment | RQ | SE* |

Relative Transcript Levels Eggs

| | | |
|---|---|---|
| kruppel | 0.40 | 0.12 (A) |
| GFP | 0.95 | 0.15 (B) |
| Water | 0.87 | 0.1 (B) |

Relative Transcript Levels Adult Females

| | | |
|---|---|---|
| kruppel | 0.06 | 0.04 (A) |
| GFP | 0.63 | 0.03 (A) |
| Water | 0.87 | 0.09 (B) |

Relative Transcript Levels Larvae

| | | |
|---|---|---|
| kruppel | 0.95 | 0.07 (A) |
| GFP | 1.06 | 0.35 (A) |
| Water | 0.70 | 0.17 (A) |

*SE—Standard Error. Letters in parentheses designate statistical levels. Levels not connected by same letter are significantly different ((p < 0.05); N = 3 biological replications of 10 eggs, larvae or adults/replication with 2 technical replications/sample).

### Example 8

### Construction of Plant Transformation Vectors

An entry vector harboring a target gene construct for dsRNA hairpin formation comprising segments of kruppel (SEQ ID NO:1 and/or SEQ ID NO:2) and/or kruppel Reg1 (SEQ ID NO:4) is assembled using a combination of chemically synthesized fragments (DNA2.0, Menlo Park, Calif.) and standard molecular cloning methods. Intramolecular hairpin formation by RNA primary transcripts is facilitated by arranging (within a single transcription unit) two copies of a target gene segment in opposite orientation to one another, the two segments being separated by a linker sequence (e.g. ST-LS1, SEQ ID NO:46; Vancanneyt et al. (1990) Mol. Gen. Genet. 220:245-250). Thus, the primary mRNA transcript contains the two kruppel gene segment sequences as large inverted repeats of one another, separated by the linker sequence. A copy of a promoter (e.g. maize ubiquitin 1, U.S. Pat. No. 5,510,474; 35S from Cauliflower Mosaic Virus (CaMV); promoters from rice actin genes; ubiquitin promoters; pEMU; MAS; maize H3 histone promoter; ALS promoter; phaseolin gene promoter; cab; rubisco; LAT52; Zm13; and/or apg) is used to drive production of the primary mRNA hairpin transcript, and a fragment comprising a 3′ untranslated region for example but not limited to a maize peroxidase 5 gene (ZmPer5 3′UTR v2; U.S. Pat. No. 6,699,984), AtUbi10, AtEf1, or StPinII is used to terminate transcription of the hairpin-RNA-expressing gene.

An entry vector is used in standard GATEWAY® recombination reactions with a typical binary destination vector to produce kruppel hairpin RNA expression transformation vectors for Agrobacterium-mediated maize embryo transformations.

A negative control binary vector which comprises a gene that expresses a YFP hairpin dsRNA, is constructed by means of standard GATEWAY® recombination reactions with a typical binary destination vector and the entry vector. The entry vector comprises a YFP hairpin sequence under the expression control of a maize ubiquitin 1 promoter (as above) and a fragment comprising a 3′ untranslated region from a maize peroxidase 5 gene (as above).

A Binary destination vector comprises a herbicide tolerance gene (aryloxyalknoate dioxygenase; (AAD-1 v3, U.S. Pat. No. 7,838,733, and Wright et al. (2010) Proc. Natl. Acad. Sci. U.S.A. 107:20240-5)) under the regulation of a plant operable promoter (e.g., sugarcane bacilliform badnavirus (ScBV) promoter (Schenk et al. (1999) Plant Mol. Biol. 39:1221-30) or ZmUbil (U.S. Pat. No. 5,510,474)). 5′ UTR and intron from these promoters, are positioned between the 3′ end of the promoter segment and the start codon of the AAD-1 coding region. A fragment comprising a 3′ untranslated region from a maize lipase gene (ZmLip 3′UTR; U.S. Pat. No. 7,179,902) is used to terminate transcription of the AAD-1 mRNA.

A further negative control binary vector that comprises a gene that expresses a YFP protein, is constructed by means of standard GATEWAY® recombination reactions with a typical binary destination vector and entry vector. The binary destination vector can comprise a herbicide tolerance gene (aryloxyalknoate dioxygenase; AAD-1 v3) (as above) under the expression regulation of a maize ubiquitin 1 promoter (as above) and a fragment comprising a 3′ untranslated region from a maize lipase gene (ZmLip 3′UTR; as above). The entry vector comprises a YFP coding region under the expression control of a maize ubiquitin 1 promoter (as above) and a fragment comprising a 3′ untranslated region from a maize peroxidase 5 gene (as above).

### Example 9

### Transgenic Maize Tissues Comprising Insecticidal dsRNAs

Agrobacterium-Mediated Transformation.

Transgenic maize cells, tissues, and plants that produce one or more insecticidal dsRNA molecules (for example, at least one dsRNA molecule including a dsRNA molecule targeting a gene comprising segments of kruppel (SEQ ID NO:1 and/or SEQ ID NO:2) and kruppel Reg1 (SEQ ID NO:4) through expression of a chimeric gene stably integrated into the plant genome are produced following Agrobacterium-mediated transformation. Maize transformation methods employing superbinary or binary transformation vectors are known in the art, as described, for example, in U.S. Pat. No. 8,304,604, which is herein incorporated by reference in its entirety. Transformed tissues are selected by their ability to grow on Haloxyfop-containing medium and are screened for dsRNA production, as appropriate. Portions of such transformed tissue cultures may be presented to neonate corn rootworm larvae for bioassay, essentially as described in EXAMPLE 1.

Agrobacterium Culture Initiation.

Glycerol stocks of Agrobacterium strain DAt13192 cells (WO 2012/016222A2) harboring a binary transformation vector pDAB109819 or pDAB114245 described above (EXAMPLE 7) are streaked on AB minimal medium plates (Watson et al. (1975) J. Bacteriol. 123:255-264) containing appropriate antibiotics and are grown at 20° C. for 3 days. The cultures are then streaked onto YEP plates (gm/L: yeast extract, 10; Peptone, 10; NaCl 5) containing the same antibiotics and were incubated at 20° C. for 1 day.

Agrobacterium Culture.

On the day of an experiment, a stock solution of Inoculation Medium and acetosyringone is prepared in a volume appropriate to the number of constructs in the experiment and pipetted into a sterile, disposable, 250 mL flask. Inoculation Medium (Frame et al. (2011) Genetic Transformation Using Maize Immature Zygotic Embryos. IN Plant Embryo Culture Methods and Protocols: Methods in Molecular Biology. T. A. Thorpe and E. C. Yeung, (Eds), Springer Science and Business Media, LLC. pp 327-341) contained: 2.2 gm/L MS salts; 1×ISU Modified MS Vitamins (Frame et al. (2011)) 68.4 gm/L sucrose; 36 gm/L glucose; 115 mg/L L-proline; and 100 mg/L myo-inositol; at pH 5.4.) Acetosyringone is added to the flask containing Inoculation Medium to a final concentration of 200 μM from a 1 M stock solution in 100% dimethyl sulfoxide and the solution is thoroughly mixed.

For each construct, 1 or 2 inoculating loops-full of Agrobacterium from the YEP plate are suspended in 15 mL of the Inoculation Medium/acetosyringone stock solution in a sterile, disposable, 50 mL centrifuge tube, and the optical density of the solution at 550 nm (OD₅₅₀) is measured in a spectrophotometer. The suspension is then diluted to OD₅₅₀ of 0.3 to 0.4 using additional Inoculation Medium/acetosyringone mixture. The tube of Agrobacterium suspension is then placed horizontally on a platform shaker set at about 75 rpm at room temperature and shaken for 1 to 4 hours while embryo dissection is performed.

Ear Sterilization and Embryo Isolation.

Maize immature embryos are obtained from plants of Zea mays inbred line B104 (Hallauer et al. (1997) Crop Science 37:1405-1406) grown in the greenhouse and self- or sib-pollinated to produce ears. The ears are harvested approximately 10 to 12 days post-pollination. On the experimental day, de-husked ears are surface-sterilized by immersion in a 20% solution of commercial bleach (ULTRA CLOROX® GERMICIDAL BLEACH, 6.15% sodium hypochlorite; with two drops of TWEEN 20) and shaken for 20 to 30 min, followed by three rinses in sterile deionized water in a laminar flow hood. Immature zygotic embryos (1.8 to 2.2 mm long) are aseptically dissected from each ear and randomly distributed into microcentrifuge tubes containing 2.0 mL of a suspension of appropriate Agrobacterium cells in liquid Inoculation Medium with 200 μM acetosyringone, into which 2 μL of 10% BREAK-THRU® 5233 surfactant (EVONIK INDUSTRIES; Essen, Germany) had been added. For a given set of experiments, embryos from pooled ears are used for each transformation.

Agrobacterium Co-Cultivation.

Following isolation, the embryos are placed on a rocker platform for 5 minutes. The contents of the tube are then poured onto a plate of Co-cultivation Medium, which contains 4.33 gm/L MS salts; 1×ISU Modified MS Vitamins; 30 gm/L sucrose; 700 mg/L L-proline; 3.3 mg/L Dicamba in KOH (3,6-dichloro-o-anisic acid or 3,6-dichloro-2-methoxybenzoic acid); 100 mg/L myo-inositol; 100 mg/L Casein Enzymatic Hydrolysate; 15 mg/L AgNO₃; 200 μM acetosyringone in DMSO; and 3 gm/L GELZAN™, at pH 5.8. The liquid Agrobacterium suspension is removed with a sterile, disposable, transfer pipette. The embryos are then oriented with the scutellum facing up using sterile forceps with the aid of a microscope. The plate is closed, sealed with 3M™ MICROPORE™ medical tape, and placed in an incubator at 25° C. with continuous light at approximately 60 μmol m⁻² s⁻¹ of Photosynthetically Active Radiation (PAR).

Callus Selection and Regeneration of Transgenic Events.

Following the Co-Cultivation period, embryos are transferred to Resting Medium, which is composed of 4.33 gm/L MS salts; 1×ISU Modified MS Vitamins; 30 gm/L sucrose; 700 mg/L L-proline; 3.3 mg/L Dicamba in KOH; 100 mg/L myo-inositol; 100 mg/L Casein Enzymatic Hydrolysate; 15 mg/L AgNO₃; 0.5 gm/L MES (2-(N-morpholino)ethanesulfonic acid monohydrate; PHYTOTECHNOLOGIES LABR.; Lenexa, Kans.); 250 mg/L Carbenicillin; and 2.3 gm/L GELZAN™; at pH 5.8. No more than 36 embryos are moved to each plate. The plates are placed in a clear plastic box and incubated at 27° C. with continuous light at approximately 50 μmol m⁻² s⁻¹ PAR for 7 to 10 days. Callused embryos are then transferred (<18/plate) onto Selection Medium I, which is comprised of Resting Medium (above) with 100 nM R-Haloxyfop acid (0.0362 mg/L; for selection of calli harboring the AAD-1 gene). The plates are returned to clear boxes and incubated at 27° C. with continuous light at approximately 50 μmol m⁻² s⁻¹ PAR for 7 days. Callused embryos are then transferred (<12/plate) to Selection Medium II, which is comprised of Resting Medium (above) with 500 nM R-Haloxyfop acid (0.181 mg/L). The plates are returned to clear boxes and incubated at 27° C. with continuous light at approximately 50 μmol m⁻² s⁻¹ PAR for 14 days. This selection step allows transgenic callus to further proliferate and differentiate.

Proliferating, embryogenic calli are transferred (<9/plate) to Pre-Regeneration medium. Pre-Regeneration Medium contains 4.33 gm/L MS salts; 1×ISU Modified MS Vitamins; 45 gm/L sucrose; 350 mg/L L-proline; 100 mg/L myo-inositol; 50 mg/L Casein Enzymatic Hydrolysate; 1.0 mg/L AgNO₃; 0.25 gm/L MES; 0.5 mg/L naphthaleneacetic acid in NaOH; 2.5 mg/L abscisic acid in ethanol; 1 mg/L 6-benzylaminopurine; 250 mg/L Carbenicillin; 2.5 gm/L GELZAN™; and 0.181 mg/L Haloxyfop acid; at pH 5.8. The plates are stored in clear boxes and incubated at 27° C. with continuous light at approximately 50 μmol m⁻² s⁻¹ PAR for 7 days. Regenerating calli are then transferred (<6/plate) to Regeneration Medium in PHYTATRAYS™ (SIGMA-ALDRICH) and incubated at 28° C. with 16 hours light/8 hours dark per day (at approximately 160 μmol m⁻² s⁻¹ PAR) for 14 days or until shoots and roots develop. Regeneration Medium contains 4.33 gm/L MS salts; 1×ISU Modified MS Vitamins; 60 gm/L sucrose; 100 mg/L myo-inositol; 125 mg/L Carbenicillin; 3 gm/L GELLAN™ gum; and 0.181 mg/L R-Haloxyfop acid; at pH 5.8. Small shoots with primary roots are then isolated and transferred to Elongation Medium without selection. Elongation Medium contains 4.33 gm/L MS salts; 1×ISU Modified MS Vitamins; 30 gm/L sucrose; and 3.5 gm/L GELRITE™: at pH 5.8.

Transformed plant shoots selected by their ability to grow on medium containing Haloxyfop are transplanted from PHYTATRAYS™ to small pots filled with growing medium (PROMIX BX; PREMIER TECH HORTICULTURE), covered with cups or HUMI-DOMES (ARCO PLASTICS), and then hardened-off in a CONVIRON™ growth chamber (27° C. day/24° C. night, 16-hour photoperiod, 50-70% RH, 200 μmol m⁻² s⁻¹ PAR). In some instances, putative transgenic plantlets are analyzed for transgene relative copy number by quantitative real-time PCR assays using primers designed to detect the AAD1 herbicide tolerance gene integrated into the maize genome. Further, RNA qPCR assays are used to detect the presence of the linker sequence in expressed dsRNAs of putative transformants. Selected transformed plantlets are then moved into a greenhouse for further growth and testing.

Transfer and Establishment of to Plants in the Greenhouse for Bioassay and Seed Production.

When plants reach the V3-V4 stage, they are transplanted into IE CUSTOM BLEND (PROFILE/METRO MIX 160) soil mixture and grown to flowering in the greenhouse (Light Exposure Type: Photo or Assimilation; High Light Limit: 1200 PAR; 16-hour day length; 27° C. day/24° C. night).

Plants to be used for insect bioassays are transplanted from small pots to TINUS™ 350-4 ROOTRAINERS® (SPENCER-LEMAIRE INDUSTRIES; Acheson, Alberta, Canada) (one plant per event per ROOTRAINER®). Approximately four days after transplanting to ROOTRAINERS®, plants are used in bioassays.

Plants of the T₁ generation are obtained by pollinating the silks of T₀ transgenic plants with pollen collected from plants of non-transgenic elite inbred line B104 or other appropriate pollen donors, and planting the resultant seeds. Reciprocal crosses are performed when possible.

### Example 10

### Adult Diabrotica Plant Feeding Bioassay

Transgenic corn foliage (V3-4) expressing dsRNA for parental RNAi targets and GFP controls is lyophilized and ground to a fine powder with mortar and pestle and sieved through a 600 μM screen in order to achieve a uniform particle size prior to incorporation into artificial diet. The artificial diet is the same diet described previously for parental RNAi experiments except that the amount of water is doubled (20 mL ddH₂O, 0.40 g agar, 6.0 g diet mix, 700 μL, glycerol, 27.5 μL, mold inhibitor). Prior to solidification, powdered corn leaf tissue is incorporated into the diet at a rate of 40 mg/ml of diet and mixed thoroughly. The diet is then poured onto the surface of a plastic petri dish to a depth of approximately 4 mm and allowed to solidify. Diet plugs are cut from the diet and used to expose western corn rootworm adults using the same methods described previously for parental RNAi experiments.

The pRNAi T₀ or T₁ events are grown in the greenhouse until the plants produce cobs, tassel and silk. A total of 25 newly emerged rootworm adults are released on each plant, and the entire plant is covered to prevent adults from escaping. Two weeks after release, female adults are recovered from each plant and maintained in the laboratory for egg collection. Depending on the parental RNAi target and expected phenotype, parameters such as number of eggs per female, percent egg hatch and larval mortality are recorded and compared with control plants.

### Example 11

### Diabrotica Larval Root-Feeding Bioassay of Transgenic Maize

Insect Bioassays.

Bioactivity of dsRNA of the subject invention produced in plant cells is demonstrated by bioassay methods. One is able to demonstrate efficacy, for example, by feeding various plant tissues or tissue pieces derived from a plant producing an insecticidal dsRNA to target insects in a controlled feeding environment. Alternatively, extracts are prepared from various plant tissues derived from a plant producing the insecticidal dsRNA and the extracted nucleic acids are dispensed on top of artificial diets for bioassays as previously described herein. The results of such feeding assays are compared to similarly conducted bioassays that employ appropriate control tissues from host plants that do not produce an insecticidal dsRNA, or to other control samples.

Insect Bioassays with Transgenic Maize Events.

Two western corn rootworm larvae (1 to 3 days old) hatched from washed eggs are selected and placed into each well of the bioassay tray. The wells are then covered with a “PULL N' PEEL” tab cover (BIO-CV-16, BIO-SERV) and placed in a 28° C. incubator with an 18 hr/6 hr light/dark cycle. Nine days after the initial infestation, the larvae are assessed for mortality, which is calculated as the percentage of dead insects out of the total number of insects in each treatment. The insect samples are frozen at −20° C. for two days, then the insect larvae from each treatment are pooled and weighed. The percent of growth inhibition is calculated as the mean weight of the experimental treatments divided by the mean of the average weight of two control well treatments. The data are expressed as a Percent Growth Inhibition (of the Negative Controls). Mean weights that exceed the control mean weight are normalized to zero.

Insect Bioassays in the Greenhouse.

Western corn rootworm (WCR, Diabrotica virgifera virgifera LeConte) eggs are received in soil from CROP CHARACTERISTICS (Farmington, Minn.). WCR eggs are incubated at 28° C. for 10 to 11 days. Eggs are washed from the soil, placed into a 0.15% agar solution, and the concentration is adjusted to approximately 75 to 100 eggs per 0.25 mL aliquot. A hatch plate is set up in a Petri dish with an aliquot of egg suspension to monitor hatch rates.

The soil around the maize plants growing in ROOTRANERS® is infested with 150 to 200 WCR eggs. The insects are allowed to feed for 2 weeks, after which time a “Root Rating” is given to each plant. A Node-Injury Scale is utilized for grading, essentially according to Oleson et al. (2005) J. Econ. Entomol. 98:1-8. Plants which pass this bioassay are transplanted to 5-gallon pots for seed production. Transplants are treated with insecticide to prevent further rootworm damage and insect release in the greenhouses. Plants are hand pollinated for seed production. Seeds produced by these plants are saved for evaluation at the T₁ and subsequent generations of plants.

Greenhouse bioassays include two kinds of negative control plants. Transgenic negative control plants are generated by transformation with vectors harboring genes designed to produce a yellow fluorescent protein (YFP) or a YFP hairpin dsRNA (See EXAMPLE 4). Non-transformed negative control plants are grown from seeds of line B104. Bioassays are conducted on two separate dates, with negative controls included in each set of plant materials.

### Example 12

### Molecular Analyses of Transgenic Maize Tissues

Molecular analyses (e.g., RNA qPCR) of maize tissues are performed on samples from leaves and roots that are collected from greenhouse grown plants on the same days that root feeding damage is assessed.

Results of RNA qPCR assays for the Per5 3′UTR are used to validate expression of hairpin transgenes. (A low level of Per5 3′UTR detection is expected in non-transformed maize plants, since there is usually expression of the endogenous Per5 gene in maize tissues.) Results of RNA qPCR assay for intervening sequence between repeat sequences (which is integral to the formation of dsRNA hairpin molecules) in expressed RNAs are used to validate the presence of hairpin transcripts. Transgene RNA expression levels are measured relative to the RNA levels of an endogenous maize gene.

DNA qPCR analyses to detect a portion of the AAD1 coding region in gDNA are used to estimate transgene insertion copy number. Samples for these analyses are collected from plants grown in environmental chambers. Results are compared to DNA qPCR results of assays designed to detect a portion of a single-copy native gene, and simple events (having one or two copies of the transgenes) are advanced for further studies in the greenhouse.

Additionally, qPCR assays designed to detect a portion of the spectinomycin-resistance gene (SpecR; harbored on the binary vector plasmids outside of the T-DNA) are used to determine if the transgenic plants contain extraneous integrated plasmid backbone sequences.

Hairpin RNA Transcript Expression Level: Per 5 3′UTR qPCR

Callus cell events or transgenic plants are analyzed by real time quantitative PCR (qPCR) of the Per 5 3′UTR sequence to determine the relative expression level of the full length hairpin transcript, as compared to the transcript level of an internal maize gene (for example, GENBANK Accession No. BT069734), which encodes a TIP41-like protein (i.e. a maize homolog of GENBANK Accession No. AT4G34270; having a tBLASTX score of 74% identity). RNA is isolated using an RNAEASY™ 96 kit (QIAGEN, Valencia, Calif.). Following elution, the total RNA is subjected to a DNaseI treatment according to the kit's suggested protocol. The RNA is then quantified on a NANODROP 8000 spectrophotometer (THERMO SCIENTIFIC) and concentration is normalized to 25 ng/μL. First strand cDNA is prepared using a HIGH CAPACITY cDNA SYNTHESIS KIT (INVITROGEN) in a 10 μL reaction volume with 5 μL denatured RNA, substantially according to the manufacturer's recommended protocol. The protocol is modified slightly to include the addition of 10 μL of 100 μM T20VN oligonucleotide (IDT) (TTTTTTTTTTTTTTTTTTTTVN, where V is A, C, or G, and N is A, C, G, or T; SEQ ID NO:47) into the 1 mL tube of random primer stock mix, in order to prepare a working stock of combined random primers and oligo dT.

Following cDNA synthesis, samples are diluted 1:3 with nuclease-free water, and stored at −20° C. until assayed.

Separate real-time PCR assays for the Per5 3′ UTR and TIP41-like transcript are performed on a LIGHTCYCLER™ 480 (ROCHE DIAGNOSTICS, Indianapolis, Ind.) in 10 μL reaction volumes. For the Per5 3′UTR assay, reactions are run with Primers P5U76S (F) (SEQ ID NO:48) and P5U76A (R) (SEQ ID NO:49), and a ROCHE UNIVERSAL PROBE™ (UPL76; Catalog No. 4889960001; labeled with FAM). For the TIP41-like reference gene assay, primers TIPmxF (SEQ ID NO:50) and TIPmxR (SEQ ID NO:51), and Probe HXTIP (SEQ ID NO:52) labeled with HEX (hexachlorofluorescein) are used.

All assays include negative controls of no-template (mix only). For standard curves, a blank (water in source well) is also included in the source plate to check for sample cross-contamination. Primer and probe sequences are set forth in Table 7. Reaction components recipes for detection of the various transcripts are disclosed in Table 8, and PCR reactions conditions are summarized in Table 9. The FAM (6-Carboxy Fluorescein Amidite) fluorescent moiety is excited at 465 nm and fluorescence is measured at 510 nm; the corresponding values for the HEX (hexachlorofluorescein) fluorescent moiety are 533 nm and 580 nm.

TABLE 7
Oligonucleotide sequences used for molecular analyses of transcript levels
in transgenic maize.

| | | SEQ ID | |
|---|---|---|---|
| Target | Oligonucleotide | NO. | Sequence |
| Per5 3′UTR | P5U76S (F) | 48 | TTGTGATGTTGGTGGCGTAT |
| Per5 3′UTR | P5U76A (R) | 49 | TGTTAAATAAAACCCCAAAGATCG |
| Per5 3′UTR | Roche UPL76 | NAv** | Roche Diagnostics Catalog Number 488996001 |
| | (FAM-Probe) | | |
| TIP41 | TIPmxF | 50 | TGAGGGTAATGCCAACTGGTT |
| TIP41 | TIPmxR | 51 | GCAATGTAACCGAGTGTCTCTCAA |
| TIP41 | HXTIP (HEX- | 52 | TTTTTGGCTTAGAGTTGATGGTGTACTGAT |
| | Probe) | | GA |

*TIP41-like protein.
**NAv Sequence Not Available from the supplier.

TABLE 8
PCR reaction recipes for transcript detection.

| | |
|---|---|
| Per5 3′UTR | TIP-like Gene |

| | |
|---|---|
| Component | Final Concentration |

| | | |
|---|---|---|
| Roche Buffer | 1 X | 1X |
| P5U76S (F) | 0.4 μM | 0 |
| P5U76A (R) | 0.4 μM | 0 |
| Roche UPL76 (FAM) | 0.2 μM | 0 |
| HEXtipZM F | 0 | 0.4 μM |
| HEXtipZM R | 0 | 0.4 μM |
| HEXtipZMP (HEX) | 0 | 0.2 μM |
| cDNA (2.0 μL) | NA | NA |
| Water | To 10 μL | To 10 μL |

TABLE 9
Thermocycler conditions for RNA qPCR.
Per5 3′UTR and TIP41-like Gene Detection

| Process | Temp. | Time | | No. Cycles |
|---|---|---|---|---|
| Target Activation | 95° C. | 10 | min | 1 |
| Denature | 95° C. | 10 | sec | 40 |
| Extend | 60° C. | 40 | sec | |
| Acquire FAM or HEX | 72° C. | 1 | sec | |
| Cool | 40° C. | 10 | sec | 1 |

Data are analyzed using LIGHTCYCLER™ Software v1.5 by relative quantification using a second derivative max algorithm for calculation of Cq values according to the supplier's recommendations. For expression analyses, expression values are calculated using the ΔΔCt method (i.e., 2-(Cq TARGET-Cq REF)), which relies on the comparison of differences of Cq values between two targets, with the base value of 2 being selected under the assumption that, for optimized PCR reactions, the product doubles every cycle.

Hairpin Transcript Size and Integrity: Northern Blot Assay.

In some instances, additional molecular characterization of the transgenic plants is obtained by the use of Northern Blot (RNA blot) analysis to determine the molecular size of the kruppel hairpin RNA in transgenic plants expressing a kruppel hairpin dsRNA.

All materials and equipment are treated with RNaseZAP (AMBION/INVITROGEN) before use. Tissue samples (100 mg to 500 mg) are collected in 2 mL SAFELOCK EPPENDORF tubes, disrupted with a KLECKO™ tissue pulverizer (GARCIA MANUFACTURING, Visalia, Calif.) with three tungsten beads in 1 mL of TRIZOL (INVITROGEN) for 5 min, then incubated at room temperature (RT) for 10 min. Optionally, the samples are centrifuged for 10 min at 4° C. at 11,000 rpm and the supernatant is transferred into a fresh 2 mL SAFELOCK EPPENDORF tube. After 200 μL chloroform are added to the homogenate, the tube is mixed by inversion for 2 to 5 min, incubated at RT for 10 minutes, and centrifuged at 12,000×g for 15 min at 4° C. The top phase is transferred into a sterile 1.5 mL EPPENDORF tube, 600 μL of 100% isopropanol are added, followed by incubation at RT for 10 min to 2 hr, and then centrifuged at 12,000×g for 10 min at 4° C. to 25° C. The supernatant is discarded and the RNA pellet is washed twice with 1 mL 70% ethanol, with centrifugation at 7,500×g for 10 min at 4° C. to 25° C. between washes. The ethanol is discarded and the pellet is briefly air dried for 3 to 5 min before resuspending in 50 μL of nuclease-free water.

Total RNA is quantified using the NANODROP8000® (THERMO-FISHER) and samples are normalized to 5 μg/10 μL. 10 μL of glyoxal (AMBION/INVITROGEN) are then added to each sample. Five to 14 ng of DIG RNA standard marker mix (ROCHE APPLIED SCIENCE, Indianapolis, Ind.) are dispensed and added to an equal volume of glyoxal. Samples and marker RNAs are denatured at 50° C. for 45 min and stored on ice until loading on a 1.25% SEAKEM GOLD agarose (LONZA, Allendale, N.J.) gel in NORTHERNMAX 10× glyoxal running buffer (AMBION/INVITROGEN). RNAs are separated by electrophoresis at 65 volts/30 mA for 2 hours and 15 minutes.

Following electrophoresis, the gel is rinsed in 2×SSC for 5 min and imaged on a GEL DOC station (BIORAD, Hercules, Calif.), then the RNA is passively transferred to a nylon membrane (MILLIPORE) overnight at RT, using 10×SSC as the transfer buffer (20×SSC consists of 3 M sodium chloride and 300 mM trisodium citrate, pH 7.0). Following the transfer, the membrane is rinsed in 2×SSC for 5 minutes, the RNA is UV-crosslinked to the membrane (AGILENT/STRATAGENE), and the membrane is allowed to dry at room temperature for up to 2 days.

The membrane is prehybridized in ULTRAHYB buffer (AMBION/INVITROGEN) for 1 to 2 hr. The probe consists of a PCR amplified product containing the sequence of interest, labeled with digoxigenin by means of a ROCHE APPLIED SCIENCE DIG procedure. Hybridization in recommended buffer is overnight at a temperature of 60° C. in hybridization tubes. Following hybridization, the blot is subjected to DIG washes, wrapped, exposed to film for 1 to 30 minutes, then the film is developed, all by methods recommended by the supplier of the DIG kit.

Transgene Copy Number Determination.

Maize leaf pieces approximately equivalent to 2 leaf punches are collected in 96-well collection plates (QIAGEN). Tissue disruption is performed with a KLECKO™ tissue pulverizer (GARCIA MANUFACTURING, Visalia, Calif.) in BIOSPRINT96 AP1 lysis buffer (supplied with a BIOSPRINT96 PLANT KIT; QIAGEN) with one stainless steel bead. Following tissue maceration, gDNA is isolated in high throughput format using a BIOSPRINT96 PLANT KIT and a BIOSPRINT96 extraction robot. gDNA is diluted 2:3 DNA:water prior to setting up the qPCR reaction.

qPCR Analysis.

Transgene detection by hydrolysis probe assay is performed by real-time PCR using a LIGHTCYCLER®480 system. Oligonucleotides to be used in hydrolysis probe assays to detect the linker sequence (e.g. ST-LS1; SEQ ID NO:46), or to detect a portion of the SpecR gene (i.e. the spectinomycin resistance gene borne on the binary vector plasmids; SEQ ID NO:53; SPC1 oligonucleotides in Table 10), are designed using LIGHTCYCLER® PROBE DESIGN SOFTWARE 2.0. Further, oligonucleotides to be used in hydrolysis probe assays to detect a segment of the AAD-1 herbicide tolerance gene (SEQ ID NO:54; GAAD1 oligonucleotides in Table 10) are designed using PRIMER EXPRESS software (APPLIED BIOSYSTEMS). Table 10 shows the sequences of the primers and probes. Assays are multiplexed with reagents for an endogenous maize chromosomal gene (Invertase; GENBANK Accession No: U16123; referred to herein as IVR1), which serves as an internal reference sequence to ensure gDNA was present in each assay. For amplification, LIGHTCYCLER®480 PROBES MASTER mix (ROCHE APPLIED SCIENCE) is prepared at 1× final concentration in a 10 μL volume multiplex reaction containing 0.4 μM of each primer and 0.2 μM of each probe (Table 11). A two-step amplification reaction is performed as outlined in Table 12. Fluorophore activation and emission for the FAM- and HEX-labeled probes are as described above; CY5 conjugates are excited maximally at 650 nm and fluoresce maximally at 670 nm.

Cp scores (the point at which the fluorescence signal crosses the background threshold) are determined from the real time PCR data using the fit points algorithm (LIGHTCYCLER® SOFTWARE release 1.5) and the Relative Quant module (based on the ΔΔCt method). Data are handled as described previously (above; RNA qPCR).

TABLE 10
Sequences of primers and probes (with fluorescent
conjugate) used for gene copy number
determinations and binary vector plasmid
backbone detection.

| | SEQ | |
|---|---|---|
| | ID | |
| Name | NO: | Sequence |
| ST-LS1-F | 55 | GTATGTTTCTGCTTCTACCTTTGAT |
| ST-LS1-R | 56 | CCATGTTTTGGTCATATATTAGAAAAGTT |
| ST-LS1-P | 57 | AGTAATATAGTATTTCAAGTATTTTTTTCAAAAT |
| (FAM) | | |
| GAAD1-F | 58 | TGTTCGGTTCCCTCTACCAA |
| GAAD1-R | 59 | CAACATCCATCACCTTGACTGA |
| GAAD1-P (FAM) | 60 | CACAGAACCGTCGCTTCAGCAACA |
| IVR1-F | 61 | TGGCGGACGACGACTTGT |
| IVR1-R | 62 | AAAGTTTGGAGGCTGCCGT |
| IVR1-P (HEX) | 63 | CGAGCAGACCGCCGTGTACTTCTACC |
| SPC1A | 64 | CTTAGCTGGATAACGCCAC |
| SPC1S | 65 | GACCGTAAGGCTTGATGAA |
| TQSPEC (CY5*) | 66 | CGAGATTCTCCGCGCTGTAGA |

CY5 = Cyanine-5

TABLE 11
Reaction components for gene copy number
analyses and plasmid backbone detection.

| | Amt. | | Final |
|---|---|---|---|
| Component | (μL) | Stock | Concentration |
| 2x Buffer | 5.0 | 2x | 1x |
| Appropriate Forward Primer | 0.4 | 10 μM | 0.4 |
| Appropriate Reverse Primer | 0.4 | 10 μM | 0.4 |
| Appropriate Probe | 0.4 | 5 μM | 0.2 |
| IVR1-Forward Primer | 0.4 | 10 μM | 0.4 |
| IVR1-Reverse Primer | 0.4 | 10 μM | 0.4 |
| IVR1-Probe | 0.4 | 5 μM | 0.2 |
| H₂O | 0.6 | NA* | NA |
| gDNA | 2.0 | ND** | ND |
| Total | 10.0 | | |

*NA = Not Applicable
**ND = Not Determined

TABLE 12
Thermocycler conditions for DNA qPCR.
Genomic copy number analyses

| Process | Temp. | Time | | No. Cycles |
|---|---|---|---|---|
| Target Activation | 95° C. | 10 | min | 1 |
| Denature | 95° C. | 10 | sec | 40 |
| Extend & Acquire | 60° C. | 40 | sec | |
| FAM, HEX, or CY5 | | | | |
| Cool | 40° C. | 10 | sec | 1 |

### Example 13

### Transgenic Zea mays Comprising Coleopteran Pest Sequences

Ten to 20 transgenic T₀ Zea mays plants are generated as described in EXAMPLE 8. A further 10-20 T₁ Zea mays independent lines expressing hairpin dsRNA for an RNAi construct are obtained for corn rootworm challenge. Hairpin dsRNA may be derived from a sequence as set forth in SEQ ID NO:1, SEQ ID NO:2, and SEQ ID NO:4. Additional hairpin dsRNAs may be derived, for example, from coleopteran pest sequences such as, for example, Caf1-180 (U.S. Patent Application Publication No. 2012/0174258), VatpaseC (U.S. Patent Application Publication No. 2012/0174259), Rhol (U.S. Patent Application Publication No. 2012/0174260), VatpaseH (U.S. Patent Application Publication No. 2012/0198586), PPI-87B (U.S. Patent Application Publication No. 2013/0091600), RPA70 (U.S. Patent Application Publication No. 2013/0091601), RPS6 (U.S. Patent Application Publication No. 2013/0097730), Hunchback (USSN), and Brahma (USSN). These are confirmed through RT-PCR or other molecular analysis methods. Total RNA preparations from selected independent T₁ lines are optionally used for RT-PCR with primers designed to bind in the linker of the hairpin expression cassette in each of the RNAi constructs. In addition, specific primers for each target gene in an RNAi construct are optionally used to amplify and confirm the production of the pre-processed mRNA required for siRNA production in planta. The amplification of the desired bands for each target gene confirms the expression of the hairpin RNA in each transgenic Zea mays plant. Processing of the dsRNA hairpin of the target genes into siRNA is subsequently optionally confirmed in independent transgenic lines using RNA blot hybridizations.

Moreover, RNAi molecules having mismatch sequences with more than 80% sequence identity to target genes affect corn rootworms in a way similar to that seen with RNAi molecules having 100% sequence identity to the target genes. The pairing of mismatch sequence with native sequences to form a hairpin dsRNA in the same RNAi construct delivers plant-processed siRNAs capable of affecting the growth, development, reproduction, and viability of feeding coleopteran pests.

In planta delivery of dsRNA, siRNA or miRNA corresponding to target genes and the subsequent uptake by coleopteran pests through feeding results in down-regulation of the target genes in the coleopteran pest through RNA-mediated gene silencing. When the function of a target gene is important at one or more stages of development, the growth, development, and reproduction of the coleopteran pest is affected, and in the case of at least one of WCR, NCR, SCR, MCR, D. balteata LeConte, D. u. tenella, and D. u. undecimpunctata Mannerheim, leads to failure to successfully infest, feed, develop, and/or reproduce, or leads to death of the coleopteran pest. The choice of target genes and the successful application of RNAi is then used to control coleopteran pests.

Phenotypic Comparison of Transgenic RNAi Lines and Nontransformed Zea mays.

Target coleopteran pest genes or sequences selected for creating hairpin dsRNA have no similarity to any known plant gene sequence. Hence it is not expected that the production or the activation of (systemic) RNAi by constructs targeting these coleopteran pest genes or sequences will have any deleterious effect on transgenic plants. However, development and morphological characteristics of transgenic lines are compared with non-transformed plants, as well as those of transgenic lines transformed with an “empty” vector having no hairpin-expressing gene. Plant root, shoot, foliage and reproduction characteristics are compared. There is no observable difference in root length and growth patterns of transgenic and non-transformed plants. Plant shoot characteristics such as height, leaf numbers and sizes, time of flowering, floral size and appearance are similar. In general, there are no observable morphological differences between transgenic lines and those without expression of target iRNA molecules when cultured in vitro and in soil in the glasshouse.

### Example 14

### Transgenic Zea mays Comprising a Coleopteran Pest Sequence and Additional RNAi Constructs

A transgenic Zea mays plant comprising a heterologous coding sequence in its genome that is transcribed into an iRNA molecule that targets an organism other than a coleopteran pest is secondarily transformed via Agrobacterium or WHISKERS™ methodologies (See Petolino and Arnold (2009) Methods Mol. Biol. 526:59-67) to produce one or more insecticidal dsRNA molecules (for example, at least one dsRNA molecule including a dsRNA molecule targeting a gene comprising SEQ ID NO:1, SEQ ID NO:2, and/or SEQ ID NO:4). Plant transformation plasmid vectors prepared essentially as described in EXAMPLE 7 are delivered via Agrobacterium or WHISKERS™-mediated transformation methods into maize suspension cells or immature maize embryos obtained from a transgenic Hi II or B104 Zea mays plant comprising a heterologous coding sequence in its genome that is transcribed into an iRNA molecule that targets an organism other than a coleopteran pest.

### Example 15

### Transgenic Zea mays Comprising an RNAi Construct and Additional Coleopteran Pest Control Sequences

A transgenic Zea mays plant comprising a heterologous coding sequence in its genome that is transcribed into an iRNA molecule that targets a coleopteran pest organism (for example, at least one dsRNA molecule including a dsRNA molecule targeting a gene comprising SEQ ID NO:1, SEQ ID NO:2, and/or SEQ ID NO:4) is secondarily transformed via Agrobacterium or WHISKERS™ methodologies (see Petolino and Arnold (2009) Methods Mol. Biol. 526:59-67) to produce one or more insecticidal protein molecules, for example, Cry1B, Cry1I, Cry2A, Cry3, Cry7A, Cry8, Cry9D, Cry14, Cry18, Cry22, Cry23, Cry34, Cry35, Cry36, Cry37, Cry43, Cry55, Cyt1A, and Cyt2C insecticidal proteins. Plant transformation plasmid vectors prepared essentially as described in EXAMPLE 7 are delivered via Agrobacterium or WHISKERS™-mediated transformation methods into maize suspension cells or immature maize embryos obtained from a transgenic B104 Zea mays plant comprising a heterologous coding sequence in its genome that is transcribed into an iRNA molecule that targets a coleopteran pest organism. Doubly-transformed plants are obtained that produce iRNA molecules and insecticidal proteins for control of coleopteran pests.

### Example 16

### pRNAi-Mediated Insect Protection

Parental RNAi that causes egg mortality or loss of egg viability brings further durability benefits to transgenic crops that use RNAi and other mechanisms for insect protection. A basic two-patch model was used to demonstrate this utility.

One patch contained a transgenic crop expressing insecticidal ingredients, and the second patch contained a refuge crop not expressing insecticidal ingredients. Eggs were oviposited in the two-modeled patches according to their relative proportions. In this example, the transgenic patch represented 95% of the landscape, and the refuge patch represented 5%. The transgenic crop expressed an insecticidal protein active against corn rootworm larvae.

Corn rootworm resistance to the insecticidal protein was modeled as monogenic, with two possible alleles; one (S) conferring susceptibility, and the other (R) conferring resistance. The insecticidal protein was modeled to cause 97% mortality of homozygous susceptible (SS) corn rootworm larvae that feed on it. There was assumed to be no mortality of corn rootworm larvae that are homozygous for the resistance allele (RR). Resistance to the insecticidal protein was assumed to be incompletely recessive, whereby the functional dominance is 0.3 (there is 67.9% mortality of larvae that are heterozygous (RS) for resistance to the protein that feed on the transgenic crop).

The transgenic crop also expressed parentally active dsRNA that, through RNA-interference (pRNAi), causes the eggs of adult female corn rootworms that are exposed to the transgenic crop to be non-viable. Corn rootworm resistance to the pRNAi was also considered to be monogenic with two possible alleles; one (X) conferring susceptibility of the adult female to RNAi, and the other (Y) conferring resistance of the adult female to RNAi. Assuming a high level of exposure to the dsRNAs, the pRNAi was modeled to cause 99.9% of eggs produced by a homozygous susceptible (XX) female to be non-viable. The model assumed that pRNAi has no effect on the viability of eggs produced by homozygous resistant (YY) females. Resistance to the dsRNA was assumed to be recessive, whereby the functional dominance is 0.01 (98.9% of eggs produced by a female that is heterozygous (XY) for resistance to dsRNA are non-viable).

In the model, there was random mating among surviving adults and random oviposition across the two patches in accordance with their relative proportions. The genotypic frequencies of viable offspring followed Mendelian genetics for a two-locus genetic system.

The effect of pRNAi required the adult females to feed on plant tissue expressing parental active dsRNA. The interference with egg development may be lower for adult females emerging from the refuge crop than from the transgenic crop; corn rootworm adults are expected to feed more extensively in the patch in which they emerged following larval development. Therefore, the relative magnitude of the pRNAi effect on female corn rootworm adults emerging from the refuge patch was varied, with the proportion of the pRNAi effect ranging from 0 (no effect of pRNAi on adult females emerging from the refuge patch) to 1 (same effect of pRNAi on adult females emerging from the refuge patch as on adult females emerging from the transgenic patch).

This model could be easily adjusted to demonstrate the situation when the effect of pRNAi is also or alternatively achieved by feeding of adult males on plant tissue expressing parental active dsRNA.

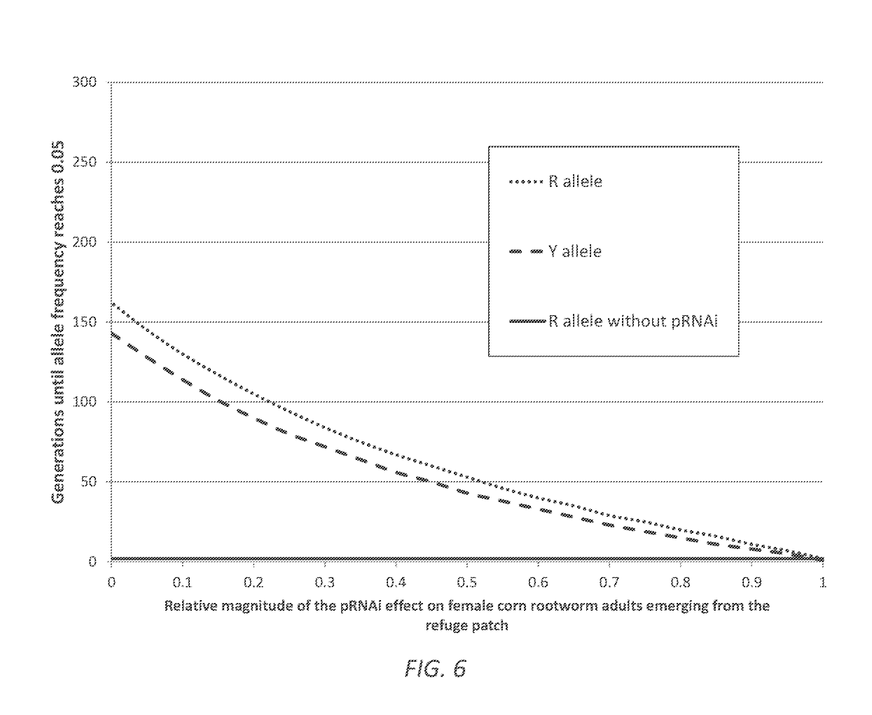

Frequencies of the two resistance alleles were calculated across generations. The initial frequencies of both of the resistance alleles (R and Y) were assumed to be 0.005. Results were presented as the number of insect generations for the frequencies of each of the resistance alleles to reach 0.05. To examine the resistance delay caused by the pRNAi, simulations that included pRNAi were compared to simulations that did not include pRNAi, but were identical in every other way. FIG. 6.

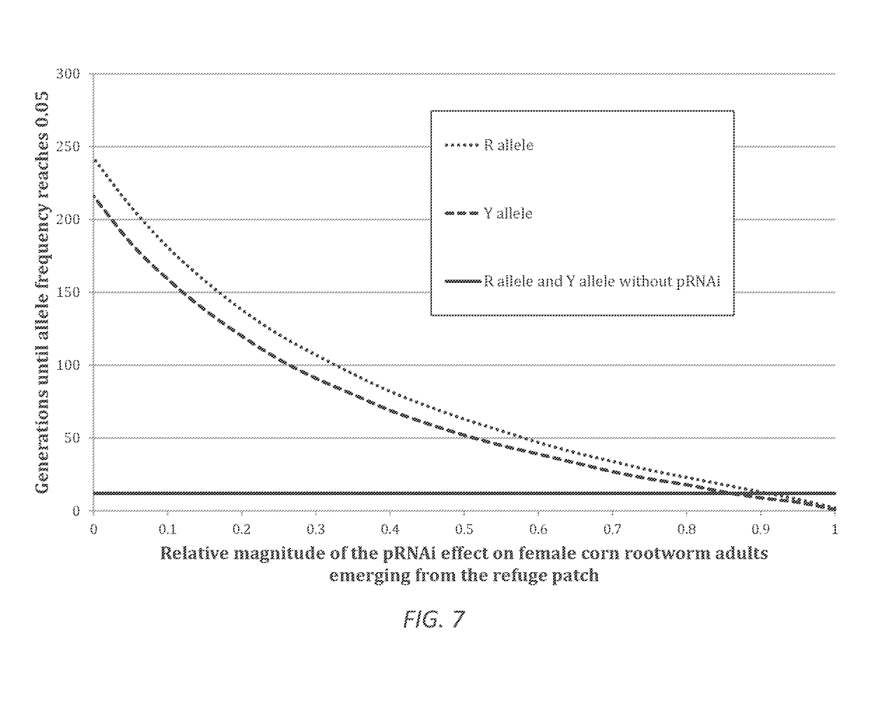

The model was also modified to include corn rootworm larval-active interfering dsRNA in combination with the corn rootworm-active insecticidal protein in the transgenic crop. Therein, the larval RNAi was assigned an effect of 97% larval mortality for homozygous RNAi-susceptible corn rootworm larvae (genotype XX), and no effect on corn rootworm larvae that are homozygous RNAi-resistant (YY). There was 67.9% mortality of corn rootworm larvae that were heterozygous for RNAi-resistance (XY). It was assumed that the same mechanism of resistance applied to both larval active RNAi and pRNAi in corn rootworms. As before, the pRNAi effect on adult females emerging from the refuge patch relative to the effect on adult females emerging from the transgenic patch was varied from 0 to 1. As before, to examine the resistance delay caused by the pRNAi, simulations that included pRNAi were compared to simulations that did not include pRNAi, but were identical in every other way (including larval RNAi). FIG. 7.

A clear resistance management benefit of pRNAi was observed when the magnitude of the pRNAi effect on egg viability for female corn rootworm adults emerging from the refuge patch was reduced compared with magnitude of the effect for adults emerging from the transgenic patch. The transgenic crops that produced parental active dsRNA in addition to an insecticidal protein were much more durable compared with transgenic crops that produced only an insecticidal protein. Similarly, transgenic crops that produced parental active dsRNA in addition to both an insecticidal protein and a larval active dsRNA were much more durable compared with transgenic crops that produced only an insecticidal protein and a larval active dsRNA. In the latter case, the durability benefit applied to both the insecticidal protein and the insecticidal interfering dsRNA.

### Example 17

### Parental RNAi Effects on WCR Males

Newly emerged virgin WCR males received from CROP CHARACTERISTICS (Farmington, Minn.) are exposed to artificial diet treated with dsRNA for pRNAi (kruppel) for 7 days with continuous dsRNA feeding. The surviving males are then paired with virgin females and allowed to mate for 4 days. Females are isolated into oviposition chambers and maintained on untreated diet to determine if mating was successful, based on egg viability. In addition, the females are dissected to determine the presence of spermatophores after 10 days of oviposition. Controls of GFP dsRNA and water are included. Three replicates of 10 males and 10 females per treatment per replication are performed. Replicates are completed with newly emerged adults on 3 different days. Each treatment per replicate contains 10 males per treatment per replication and re placed in one well of a tray. Each well includes 12 diet plugs treated with water or dsRNA (GFP or kruppel). Each diet plug is treated with 2 μg dsRNA in 3 μL water. Trays are transferred to a growth chamber with a temperature of 23±1° C., relative humidity>80%, and L:D 16:8. Males are transferred to new trays with 12 treated diet plugs in each well on days 3, 5, and 7. On day 7, three males per replication per treatment are flash frozen for qPCR analysis as described in EXAMPLE 7. On day 8, ten females and ten treated males are placed together in a container to allow mating. Each container includes 22 untreated diet plugs. Insects are transferred to new trays with 22 untreated diet plugs on day 10, and males are removed on day 12 and used to measure sperm viability using fluorescent staining techniques. Females are transferred to a new tray with 12 untreated diet plugs every other day until day 22. On day 16, females are transferred to egg cages containing autoclaved soil for oviposition. On day 22, all females are removed from the soil cages and frozen to check for the presence of spermatophores. The soil cages are transferred to a new growth chamber with a temperature of 27±1° C., relative humidity>80%, and 24 h dark. On day 28, the soil is washed using a sieve #60 to collect eggs from each cage. Eggs are treated with a solution of formaldehyde (500 μL formaldehyde in 5 mL double distilled water) and methyl-(butycarbamoy)-2-benzimidazole carbamate (0.025 g in 50 mL double distilled water) to prevent fungal contamination and are placed in small petri dishes containing filter paper. Petri dishes with eggs are transferred to a small growth chamber with a temperature of 27±1° C., relative humidity>80%, and 24 h dark. From days 29-42 larval hatch was monitored daily.

Sperm Viability.

Virgin Western corn rootworm males are exposed to artificial diet treated with dsRNA for 7 days with the parental RNAi gene kruppel. Treated diet is provided every other day. Four males per treatment per replications are used to test for sperm viability using a fluorescent technique to discriminate between living and dead sperm as described by Collins and Donoghue (1999). The Live Dead Sperm Viability Kit™ (Life Technologies, Carlsbad Calif.) contains SYBR 14, a membrane-permeant nucleic acid stain, and propidium iodine, which stains dead cells.

WCR males are anesthetized on ice, testes and seminal vesicles are dissected, placed in 10 μL buffer (HEPES 10 mM, NaCl 150 mM, BSA 10%, pH 7.4) and crushed with an autoclaved toothpick. Sperm viability is immediately assessed using the Live Dead Sperm Viability Kit™. 1 μL SYBR 14 (0.1 mM in DMSO) is added and incubated at room temperature for 10 minutes, followed by 1 μL propidium iodine (2.4 mM) and incubated again at room temperature for 10 minutes. 10 μL sperm stained solution is transferred to a glass microslide and covered with a slipcover. Samples are evaluated using a Nikon™ Eclipse 90i microscope with a Nikon A1 confocal and NIS-Elements Software. Samples are visualized at 10× with 488 excitation, a 500-550 nm band pass for live sperm (SYBR 14) and 663-738 nm band pass for dead sperm (propidium iodine) simultaneously. Digital images are recorded for five fields of view per sample. The number of live (green) and dead (red) sperm is evaluated using the cell counter function of Image J Software. Schneider et al. (2012) Nat. Methods 9:671-5.

### Example 18

### Effective Concentration

Mated females are exposed to 4 doses of kruppel dsRNA to determine the effective concentrations. Newly emerged (24-48 hours) adult males and females are received from CROP CHARACTERISTICS (Farmington, Minn.). Treatments are 2, 0.2, 0.02, and 0.002 μg kruppel dsRNA. GFP at 2 μg and water serve as the controls. Ten males and 10 females are placed together in one well containing 20 pellets of untreated artificial diet. Trays are transferred to a growth chamber and maintained at 23±1° C., relative humidity>80%, and 16:8 L:D photoperiod. Insects are transferred to new trays with new untreated artificial diet (11 plugs per well) and placed back in the growth chamber. Males are removed from the experiment on day 5. Females are transferred every other day from day 7-13 to new trays containing 11 diet plugs with the respective treatments of dsRNA. On day 14 females are transferred to egg cages containing autoclaved soil and new treated artificial diet is provided (11 plugs per cage). Egg cages are placed back in the growth chamber. On day 16 new treated diet is provided as described above. All females are removed from the soil cages on day 18 and flash frozen for RT-qPCR. Soil cages are transferred to a new growth chamber with a temperature of 27±1° C., relative humidity>80% and 24 h dark. On day 24 the soil is washed using a #60 sieve to collect eggs from each cage. Eggs are treated with a solution of formaldehyde (500 μl formaldehyde in 5 ml of double distilled water) and methyl-(butycarbamoy)-2-benzimidazole carbamate (0.025 g in 50 ml of double distilled water) to prevent fungal contamination and placed in small petri dishes containing filter paper. Photographs are taken of each petri dish for egg counting using the cell counter function of Image J software (Schneider et al. (2012) Nat. Methods 9:671-5). Petri dishes with eggs are transferred to a small growth chamber with a temperature of 27±1° C., relative humidity>80%, and 24 h dark. Larval hatching is monitored daily through 38 days. Larvae are counted and removed from the Petri dish each day.

### Example 19

### Timing of Exposure

Females are exposed to 2 μg of kruppel dsRNA starting at three different times to determine the timing of exposure necessary to generate a parental RNAi effect. Females are exposed to dsRNA 6 times before mating, 6 times immediately after mating, and 6 times 6 days after mating. Three replications of 10 females and 10 males per replication are completed for each exposure time. Females are received from CROP CHARACTERISTICS (Farmington, Minn.).

dsRNA Feeding Before Mating:

Ten females are placed in one well with 11 pellets of treated artificial diet (2 μg dsRNA per pellet). Trays are transferred to a growth chamber with a temperature of 23±1° C., relative humidity>80%, and 16:8 L:D photoperiod. Females are transferred to trays containing fresh treated diet every other day for 10 days. On day 12 females are paired with 10 males and 22 plugs of untreated diet are provided. Males are removed after 4 days. Fresh untreated diet is provided every other day for 8 days. On day 22 females are transferred to egg cages containing autoclaved soil with 11 plugs of untreated artificial diet. Egg cages are placed back in the growth chamber. The diet is replaced on day 24. On day 26 females are removed from the soil cages and flash frozen for RT-qPCR. Soil cages are transferred to a growth chamber with temperature 27±1° C., relative humidity>80% and 24 h dark. After 4 days the soil is washed using a #60 sieve to collect eggs from each cage. Eggs are treated as described above to prevent fungal contamination and placed in small petri dishes containing filter paper. Photographs are taken of each petri dish for egg counting using the cell counter function of Image J software (Schneider et al. (2012) Nat. Methods 9:671-5). Petri dishes with eggs are transferred to a small growth chamber with temperature 27±1° C., relative humidity>80%, and 24 h dark. Larval hatching is monitored daily from days 33-47. Larvae are counted and removed from the Petri dish each day.

dsRNA Feeding Immediately after Mating:

Methods similar to those described above are used except that 10 males and 10 females are placed together in one well with 22 pellets of untreated artificial diet at the start of the study. Trays are transferred to growth chamber as described above. Fresh untreated diet is provided on day 3 and males are removed on day 5. The females are transferred to treated artificial diet and maintained in the growth chamber. Fresh treated diet is provided every other day for 6 days. On day 12 females are transferred to egg cages containing autoclaved soil with 11 plugs of treated artificial diet. Egg cages are placed back in the growth chamber. Fresh treated diet is provided on day 14. On day 16 all females are removed from the soil cages and flash frozen for RT-qPCR. Soil cages and egg wash is conducted after 6 days as described above. Larval hatching is monitored daily from days 23 to 37. Larvae are counted and removed from the Petri dish each day.

dsRNA Feeding after Mating:

Methods similar to those described above for dsRNA feeding immediately after mating except that insects receive untreated artificial diet every other day until day 11 when females are transferred to treated diet. On day 12 females are transferred to egg cages containing autoclaved soil with 11 plugs of treated artificial diet. Egg cages are placed back in the growth chamber. Fresh treated diet is provided every other day from days 12-20. At day 22 all females are removed from the soil cages and flash frozen for RT-qPCR. Soil cages and egg wash is conducted after 6 days as described above. Larval hatching is monitored daily from days 29-43. Larvae are counted and removed from the Petri dish each day. Female mortality is recorded every other day for all treatments throughout the study.

### Example 20

### Duration of Exposure

Virgin males and females are paired for a period of 4 days with untreated diet after which the mated females are exposed to kruppel dsRNA. To evaluate the effect of the duration of exposure insects are exposed to 2 μg kruppel or GFP dsRNA 1, 2, 4 or 6 times. Four replications of 10 females and 10 males are completed per treatment. Adult males and females are received from CROP CHARACTERISTICS (Farmington, Minn.). Ten males and 10 females are placed together in one well with 20 pellets of untreated artificial diet. Trays are maintained in a growth chamber with a temperature of 23±1° C., relative humidity>80%, and 16:8 L:D photoperiod. New untreated artificial diet is provided on day 3. Males are removed on day 5 and females are transferred to a new tray containing the respective treatment of dsRNA on 11 diet plugs per well. On day 7 females are transferred to new trays with new treated artificial diet (11 plugs per well) and mortality is recorded. Females from one-time exposure treatment are transferred to untreated diet. On day 10 and 12 females are transferred to new trays with new treated artificial diet (11 plugs per well) and mortality is recorded. Females from 1 and 2 exposure times are transferred to untreated diet. On day 14 females are transferred to egg cages containing autoclaved soil. New treated artificial diet is provided (11 plugs per cage). Females from 1, 2 and 4 times of exposure are provided untreated diet. On day 16, old diet is removed and new treated diet (11 plugs per cage) is added. Females from 1, 2 and 4 exposure times are provided untreated diet. After 18 days all females are removed from the soil cages and flash frozen for RT-qPCR. Soil cages are transferred to a growth chamber with a temperature of 27±1° C., relative humidity>80% and 24 h dark. Soil is washed on day 24 using a #60 sieve to collect eggs from each cage. Eggs are treated as described above to prevent fungal contamination and placed in small petri dishes containing filter paper. Photographs are taken of each petri dish for egg counting using the cell counter function of Image J software (Schneider et al. (2012) Nat. Methods 9:671-5). Petri dishes with eggs are transferred to a small growth chamber with a temperature of 27±1° C., relative humidity>80%, and 24 h dark. Larval hatching is monitored daily from days 25-38. Emerging larvae are counted and removed from each petri dish daily.

What may be claimed is:

1. An isolated nucleic acid comprising at least one polynucleotide selected from the group consisting of:
SEQ ID NO:1, the complement of SEQ ID NO:1, a fragment of at least 15 contiguous nucleotides of SEQ ID NO:1, the complement of a fragment of at least 15 contiguous nucleotides of SEQ ID NO:1, a native coding sequence of a Diabrotica organism comprising SEQ ID NO:1, the complement of a native coding sequence of a Diabrotica organism comprising SEQ ID NO:1, a native non-coding sequence of a Diabrotica organism that is transcribed into a native RNA molecule comprising SEQ ID NO:67, the complement of a native non-coding sequence of a Diabrotica organism that is transcribed into a native RNA molecule comprising SEQ ID NO:67, a fragment of at least 15 contiguous nucleotides of a native coding sequence of a Diabrotica organism comprising SEQ ID NO:1, the complement of a fragment of at least 15 contiguous nucleotides of a native coding sequence of a Diabrotica organism comprising SEQ ID NO:1, a fragment of at least 15 contiguous nucleotides of a native non-coding sequence of a Diabrotica organism that is transcribed into a native RNA molecule comprising SEQ ID NO:67, and the complement of a fragment of at least 15 contiguous nucleotides of a native non-coding sequence of a Diabrotica organism that is transcribed into a native RNA molecule comprising SEQ ID NO:67;
SEQ ID NO:2, the complement of SEQ ID NO:2, a fragment of at least 15 contiguous nucleotides of SEQ ID NO:2, the complement of a fragment of at least 15 contiguous nucleotides of SEQ ID NO:2, a native coding sequence of a Diabrotica organism comprising SEQ ID NO:2, the complement of a native coding sequence of a Diabrotica organism comprising SEQ ID NO:2, a native non-coding sequence of a Diabrotica organism that is transcribed into a native RNA molecule comprising SEQ ID NO:68, the complement of a native non-coding sequence of a Diabrotica organism that is transcribed into a native RNA molecule comprising SEQ ID NO:68, a fragment of at least 15 contiguous nucleotides of a native coding sequence of a Diabrotica organism comprising SEQ ID NO:2, the complement of a fragment of at least 15 contiguous nucleotides of a native coding sequence of a Diabrotica organism comprising SEQ ID NO:2, a fragment of at least 15 contiguous nucleotides of a native non-coding sequence of a Diabrotica organism that is transcribed into a native RNA molecule comprising SEQ ID NO:68, and the complement of a fragment of at least 15 contiguous nucleotides of a native non-coding sequence of a Diabrotica organism that is transcribed into a native RNA molecule comprising SEQ ID NO:68.

2. The polynucleotide of claim 1, wherein the polynucleotide is selected from the group consisting of SEQ ID NO:1, SEQ ID NO:2, and SEQ ID NO:4.

3. A plant transformation vector comprising the polynucleotide of claim 1.

4. The polynucleotide of claim 1, wherein the organism is selected from the group consisting of D. v. virgifera LeConte; D. barberi Smith and Lawrence; D. u. howardi; D. v. zeae; D. balteata LeConte; D. u. tenella; D. speciosa Germar; and D. u. undecimpunctata Mannerheim.

5. A ribonucleic acid (RNA) molecule transcribed from the polynucleotide of claim 1.

6. A double-stranded ribonucleic acid molecule produced from the expression of the polynucleotide of claim 1.

7. The double-stranded ribonucleic acid molecule of claim 6, wherein contacting the polynucleotide sequence with a coleopteran pest inhibits the expression of an endogenous nucleotide sequence specifically complementary to the polynucleotide.

8. The double-stranded ribonucleic acid molecule of claim 7, wherein contacting said ribonucleotide molecule with a coleopteran pest kills or inhibits the growth, reproduction, and/or feeding of the pest.

9. The double stranded RNA of claim 6, comprising a first, a second and a third RNA segment, wherein the first RNA segment comprises the polynucleotide, wherein the third RNA segment is linked to the first RNA segment by the second polynucleotide sequence, and wherein the third RNA segment is substantially the reverse complement of the first RNA segment, such that the first and the third RNA segments hybridize when transcribed into a ribonucleic acid to form the double-stranded RNA.

10. The RNA of claim 5, selected from the group consisting of a double-stranded ribonucleic acid molecule and a single-stranded ribonucleic acid molecule of between about 15 and about 30 nucleotides in length.

11. A plant transformation vector comprising the polynucleotide of claim 1, wherein the heterologous promoter is functional in a plant cell.

12. A cell transformed with the polynucleotide of claim 1.

13. The cell of claim 12, wherein the cell is a prokaryotic cell.

14. The cell of claim 12, wherein the cell is a eukaryotic cell.

15. The cell of claim 14, wherein the cell is a plant cell.

16. A plant transformed with the polynucleotide of claim 1.

17. A seed of the plant of claim 16, wherein the seed comprises the polynucleotide.

18. A commodity product produced from the plant of claim 16, wherein the commodity product comprises a detectable amount of the polynucleotide.

19. The plant of claim 16, wherein the at least one polynucleotide is expressed in the plant as a double-stranded ribonucleic acid molecule.

20. The cell of claim 15, wherein the cell is a Zea mays cell.

21. The plant of claim 16, wherein the plant is Zea mays.

22. The plant of claim 16, wherein the at least one polynucleotide is expressed in the plant as a ribonucleic acid molecule, and the ribonucleic acid molecule inhibits the expression of an endogenous polynucleotide that is specifically complementary to the at least one polynucleotide when a coleopteran pest ingests a part of the plant.

23. The polynucleotide of claim 1, further comprising at least one additional polynucleotide that encodes an RNA molecule that inhibits the expression of an endogenous pest gene.

24. The polynucleotide of claim 23, wherein the additional polynucleotide encodes an iRNA molecule that results in a parental RNAi phenotype.

25. The polynucleotide of claim 24, wherein the additional polynucleotide encodes an iRNA molecule that inhibits the expression of a Brahma or hunchback gene.

26. The polynucleotide of claim 23, wherein the additional polynucleotide encodes an iRNA molecule that results in decreased growth and/or development and/or mortality in a coleopteran pest that contacts the iRNA molecule (lethal RNAi).

27. A plant transformation vector comprising the polynucleotide of claim 23, wherein the additional polynucleotide(s) are each operably linked to a heterologous promoter functional in a plant cell.

28. The polynucleotide of claim 1, wherein the polynucleotide is operably linked to a heterologous promoter

29. A method for controlling a coleopteran pest population, the method comprising providing an agent comprising a ribonucleic acid (RNA) molecule that functions upon contact with the coleopteran pest to inhibit a biological function within the coleopteran pest, wherein the RNA is specifically hybridizable with a polynucleotide selected from the group consisting of any of SEQ ID NOs:67-69; the complement of any of SEQ ID NOs:67-69; a fragment of at least 15 contiguous nucleotides of any of SEQ ID NOs:67-69; the complement of a fragment of at least 15 contiguous nucleotides of any of SEQ ID NOs:67-69; a transcript of either of SEQ ID NOs:1 and 2; the complement of a transcript of either of SEQ ID NOs:1 and 2; a fragment of at least 15 contiguous nucleotides of a transcript of either of SEQ ID NOs:1 and 2; and the complement of a fragment of at least 15 contiguous nucleotides of a transcript of either of SEQ ID NOs:1 and 2.

30. The method according to claim 29, wherein the agent is a double-stranded RNA molecule.

31. A method for controlling a coleopteran pest population, the method comprising:
introducing into a coleopteran pest, a ribonucleic acid (RNA) molecule that functions upon contact with the coleopteran pest to inhibit a biological function within the coleopteran pest, wherein the RNA is specifically hybridizable with a polynucleotide selected from the group consisting of any of
SEQ ID NOs:67-69,
the complement of any of SEQ ID NOs:67-69,
a fragment of at least 15 contiguous nucleotides of any of SEQ ID NOs:67-69,
the complement of a fragment of at least 15 contiguous nucleotides of any of SEQ ID NOs:67-69,
a transcript of either of SEQ ID NOs:1 and 2,
the complement of a transcript of either of SEQ ID NOs:1 and 2,
a fragment of at least 15 contiguous nucleotides of a transcript of either of SEQ ID NOs:1 and 2, and
the complement of a fragment of at least 15 contiguous nucleotides of a transcript of either of SEQ ID NOs:1 and 2,
thereby producing a coleopteran pest having a pRNAi phenotype.

32. The method according to claim 31, wherein the RNA is introduced into a male coleopteran pest.

33. The method according to claim 31, wherein the RNA is introduced into a female coleopteran pest, the method further comprising releasing the female coleopteran pest having the pRNAi phenotype into the pest population, wherein mating between the female coleopteran pest having the pRNAi phenotype and male pests of the population produces fewer viable offspring than mating between other female pests and male pests of the population.

34. A method for controlling a coleopteran pest population, the method comprising:
providing an agent comprising a first and a second polynucleotide sequence that functions upon contact with the coleopteran pest to inhibit a biological function within the coleopteran pest, wherein the first polynucleotide sequence comprises a region that exhibits from about 90% to about 100% sequence identity to from about 19 to about 30 contiguous nucleotides of SEQ ID NO:67 and/or SEQ ID NO:68, and wherein the first polynucleotide sequence is specifically hybridized to the second polynucleotide sequence.

35. The method according to claim 34, wherein the ribonucleic acid molecule is a double-stranded ribonucleic acid molecule.

36. The method according to claim 3343, wherein the coleopteran pest population is reduced relative to a population of the same pest species infesting a host plant of the same host plant species lacking the transformed plant cell.

37. A method for controlling a coleopteran pest population, the method comprising:
providing in a host plant of a coleopteran pest a transformed plant cell comprising the polynucleotide of claim 1, wherein the polynucleotide is expressed to produce a ribonucleic acid molecule that functions upon contact with a coleopteran pest belonging to the population to inhibit the expression of a target sequence within the coleopteran pest and results in decreased reproduction of the coleopteran pest or pest population, relative to reproduction of the same pest species on a plant of the same host plant species that does not comprise the polynucleotide.

38. The method according to claim 37, wherein the ribonucleic acid molecule is a double-stranded ribonucleic acid molecule.

39. The method according to claim 37, wherein the coleopteran pest population is reduced relative to a coleopteran pest population infesting a host plant of the same species lacking the transformed plant cell.

40. A method of controlling coleopteran pest infestation in a plant, the method comprising providing in the diet of a coleopteran pest a ribonucleic acid (RNA) that is specifically hybridizable with a polynucleotide selected from the group consisting of:
SEQ ID NOs:67-69;
the complement of any of SEQ ID NOs:67-69;
a fragment of at least 15 contiguous nucleotides of any of SEQ ID NOs:67-69;
the complement of a fragment of at least 15 contiguous nucleotides of any of SEQ ID NOs:67-69;
a transcript of either of SEQ ID NOs:1 and 2;
the complement of a transcript of either of SEQ ID NOs:1 and 2;
a fragment of at least 15 contiguous nucleotides of a transcript of either of SEQ ID NOs:1 and 2; and
the complement of a fragment of at least 15 contiguous nucleotides of a transcript of either of SEQ ID NOs:1 and 2.

41. The method according to claim 40, wherein the diet comprises a plant cell transformed to express the polynucleotide.

42. The method according to claim 40, wherein the specifically hybridizable RNA is comprised in a double-stranded RNA molecule.

43. A method for improving the yield of a corn crop, the method comprising:
introducing the nucleic acid of claim 1 into a corn plant to produce a transgenic corn plant; and
cultivating the corn plant to allow the expression of the at least one polynucleotide; wherein expression of the at least one polynucleotide inhibits coleopteran pest reproduction or growth and loss of yield due to coleopteran pest infection.

44. The method according to claim 43, wherein expression of the at least one polynucleotide produces an RNA molecule that suppresses at least a first target gene in a coleopteran pest that has contacted a portion of the corn plant.

45. A method for producing a transgenic plant cell, the method comprising:
transforming a plant cell with a vector comprising the nucleic acid of claim 1;
culturing the transformed plant cell under conditions sufficient to allow for development of a plant cell culture comprising a plurality of transformed plant cells;
selecting for transformed plant cells that have integrated the at least one polynucleotide into their genomes;
screening the transformed plant cells for expression of a ribonucleic acid (RNA) molecule encoded by the at least one polynucleotide; and
selecting a plant cell that expresses the RNA.

46. The method according to claim 45, wherein the RNA molecule is a double-stranded RNA molecule.

47. A method for providing protection against a coleopteran pest to a transgenic plant, the method comprising:
providing the transgenic plant cell produced by the method of claim 45; and
regenerating a transgenic plant from the transgenic plant cell, wherein expression of the ribonucleic acid molecule encoded by the at least one polynucleotide is sufficient to modulate the expression of a target gene in a coleopteran pest that contacts the transformed plant.

48. A method for producing a transgenic plant cell, the method comprising:
transforming a plant cell with a vector comprising a means for protecting a plant from a coleopteran pest;
culturing the transformed plant cell under conditions sufficient to allow for development of a plant cell culture comprising a plurality of transformed plant cells;
selecting for transformed plant cells that have integrated the means for protecting a plant from a coleopteran pest into their genomes;
screening the transformed plant cells for expression of a means for inhibiting expression of an essential gene in a coleopteran pest; and
selecting a plant cell that expresses the means for inhibiting expression of an essential gene in a coleopteran pest.

49. A method for producing a coleopteran pest-protected transgenic plant, the method comprising:
providing the transgenic plant cell produced by the method of claim 48; and
regenerating a transgenic plant from the transgenic plant cell, wherein expression of the means for inhibiting expression of an essential gene in a coleopteran pest is sufficient to modulate the expression of a target gene in a coleopteran pest that contacts the transformed plant.

50. The nucleic acid of claim 1, further comprising a polynucleotide encoding a polypeptide from Bacillus thuringiensis, Alcaligenes spp., or Pseudomonas spp.

51. The nucleic acid of claim 49, wherein the polypeptide from B. thuringiensis is selected from a group comprising Cry1B, Cry1I, Cry2A, Cry3, Cry7A, Cry8, Cry9D, Cry14, Cry18, Cry22, Cry23, Cry34, Cry35, Cry36, Cry37, Cry43, Cry55, Cyt1A, and Cyt2C.

52. The cell of claim 15, wherein the cell comprises a polynucleotide encoding a polypeptide from Bacillus thuringiensis, Alcaligenes spp., or Pseudomonas spp.

53. The cell of claim 51, wherein the polypeptide from B. thuringiensis is selected from a group comprising Cry1B, Cry1I, Cry2A, Cry3, Cry7A, Cry8, Cry9D, Cry14, Cry18, Cry22, Cry23, Cry34, Cry35, Cry36, Cry37, Cry43, Cry55, Cyt1A, and Cyt2C.

54. The plant of claim 16, wherein the plant comprises a polynucleotide encoding a polypeptide from Bacillus thuringiensis, Alcaligenes spp., or Pseudomonas spp.

55. The plant of claim 54, wherein the polypeptide from B. thuringiensis is selected from a group comprising Cry1B, Cry1I, Cry2A, Cry3, Cry7A, Cry8, Cry9D, Cry14, Cry18, Cry22, Cry23, Cry34, Cry35, Cry36, Cry37, Cry43, Cry55, Cyt1A, and Cyt2C.

56. The method according to claim 43, wherein the transformed plant cell comprises a nucleotide sequence encoding a polypeptide from Bacillus thuringiensis, Alcaligenes spp., or Pseudomonas spp.

57. The method according to claim 56, wherein the polypeptide from B. thuringiensis is selected from a group comprising Cry1B, Cry1I, Cry2A, Cry3, Cry7A, Cry8, Cry9D, Cry14, Cry18, Cry22, Cry23, Cry34, Cry35, Cry36, Cry37, Cry43, Cry55, Cyt1A, and Cyt2C.
